(19) United States
(12) Patent Application Publication
(10) Pub. No.: US 2021/0145860 A1
(43) Pub. Date: May 20, 2021

(54) COMPOSITIONS, METHODS AND USES OF MESSENGER RNA

(71) Applicant: Translate Bio, Inc., Lexington, MA (US)

(72) Inventors: Richard Wooster, Lexington, MA (US); Anusha Dias, Lexington, MA (US); Dustin Cooper, Lexington, MA (US); Christian Cobaugh, Lexington, MA (US); Frank DeRosa, Lexington, MA (US); Tim Efthymiou, Lexington, MA (US); Jeffrey S. Dubins, Lexington, MA (US)

(21) Appl. No.: 17/076,195

(22) Filed: Oct. 21, 2020

Related U.S. Application Data
(60) Provisional application No. 62/923,711, filed on Oct. 21, 2019.
(60) Provisional application No. 62/934,842, filed on Nov. 13, 2019.
(60) Provisional application No. 63/084,422, filed on Sep. 28, 2020.

(51) Int. Cl. A61K 31/7105; A61K 47/54; A61K 47/60; A61K 47/69

(52) U.S. Cl. CPC A61K 31/7105; A61K 47/6929; A61K 47/60; A61K 47/543

(57) ABSTRACT

The present invention provides, among other things, methods and compositions for selective degradation of proteins. In some aspects, messenger RNAs (mRNAs) are described that encode a ubiquitin pathway moiety and a binding peptide that binds a target protein, wherein the mRNA is encapsulated within a lipid nanoparticle. Also provided herein are mRNAs that encode at least two binding peptides, wherein a first binding peptide binds a ubiquitin pathway moiety and a second binding peptide binds a target protein, and wherein the mRNA is encapsulated within a lipid nanoparticle.

### CROSS-REFERENCE TO RELATED APPLICATIONS

The present application is a claims priority to U.S. Provisional Application Ser. No. 62/923,711 filed Oct. 21, 2019, U.S. Provisional Application Ser. No. 62/934,842 filed Nov. 13, 2019, and U.S. Provisional Application Ser. No. 63/084,422 filed Sep. 28, 2020, the disclosures of each of which are hereby incorporated by reference in its entirety.

### INCORPORATION-BY-REFERENCE OF SEQUENCE LISTING

This instant application contains a Sequence Listing which has been submitted electronically in ASCII format and is hereby incorporated by reference in its entirety. Said ASCII copy, created on Oct. 20, 2020, is named MRT-2120US_ST25.txt and is 20 KB in size. No new matter is hereby added.

### BACKGROUND

Degradation of cellular proteins is required for normal maintenance of cellular function, including proliferation, differentiation, and cell death. The irreversible nature of proteolysis makes it well suited to serve as a regulatory switch for controlling unidirectional processes. This principle is evident in the control of the cell cycle, where initiation of DNA replication, chromosome segregation, and exit from mitosis are triggered by the destruction of key regulatory proteins.

One of the major pathways to regulate proteins post-translationally is ubiquitin-dependent proteolysis. The first step in selective degradation is the ligation of one or more ubiquitin molecules to a protein substrate. Ubiquitination occurs through the activity of ubiquitin-activating enzymes (E1), ubiquitin-conjugating enzymes (E2), and ubiquitin-protein ligases (E3), which act sequentially to catalyze the attachment of ubiquitin to lysine residues of substrate proteins (see Ciechanover A., et al., BioEssays, 22:442-451 (2000)). The E3 protein ligases confer specificity to ubiquitination reactions by binding directly to substrate.

Many diseases and disorders are caused by the aberrant expression of proteins. Targeting such aberrantly expressed proteins for degradation is therefore a promising therapeutic approach to tackle a wide variety of diseases or disorders. However, the exploitation of the cell's own system for selective protein degradation has so far been restricted to a limited number of target proteins for which there are known small molecules or peptides that bind these proteins with high specificity to make selective protein degradation feasible. Typically, such proteins or peptides are linked to a ligase binding molecule (e.g., another small molecule or peptide). However, effective delivery of such small-molecule or peptide-based constructs to their intracellular target proteins is difficult and severely limits the size of constructs that can be delivered. Therefore, there is a need in the art to provide improved methods and compositions useful for selective protein degradation.

### SUMMARY OF INVENTION

The present invention provides an mRNA-based composition and method for selective degradation of a target protein of interest. In particular, compositions and methods described herein provide effective in vivo delivery of mRNAs encoding, among other things, ubiquitin pathway moieties and binding proteins that result in the degradation of a target protein. In some aspects, the compositions and methods described herein provide effective in vivo delivery of mRNAs encoding, among other things, at least two binding peptides, a first binding peptide that binds a ubiquitin pathway moiety and a second binding peptide that binds a target protein, wherein binding to the target protein causes selective degradation of the target protein. The mRNA-based composition and method described herein has several advantages over other compositions and methods (such as siRNA) of selective target degradation. Such advantage include for example, rapid targeting of the protein of interest for degradation, transient degradation effect, and the ease of delivery of the compositions described herein. Further advantages include the ability to target a desired protein for degradation based on its posttranslational modification status.

In one aspect, the present invention provides, among other things, a messenger RNA (mRNA) that encodes a ubiquitin pathway moiety and a binding peptide that binds a target protein, wherein the mRNA is encapsulated within a lipid nanoparticle. In some embodiments, the ubiquitin pathway moiety and the binding peptide create a fusion protein. For example, in some embodiments, the mRNA that encodes both a ubiquitin pathway moiety and the binding peptide that binds a target protein create a fusion peptide. In some embodiments, the fusion protein comprises an internal ribosome entry site (IRES). In some embodiments, at least two mRNAs are provided, in which a first mRNA encodes a ubiquitin pathway moiety, and a second mRNA encodes a binding peptide that binds a target protein.

In some embodiments, a ubiquitin pathway moiety is an E3-ubiquitin ligase, E3 ligase adaptor, or a protein or peptide that is able to induce ubiquitin-proteasome pathway.

In some embodiments, a binding peptide specifically recognizes and binds a target protein for degradation.

In some embodiments, the mRNA that encodes a ubiquitin pathway moiety and a binding peptide that binds a target protein degrades the target protein in a concentration-dependent manner.

In some embodiments, the ubiquitin pathway moiety and the binding peptide are separated by a linker.

In some embodiments, the ubiquitin pathway moiety is a ubiquitin pathway protein.

In some embodiments, the linker is a GS linker. For example, in some embodiments, the GS linker comprises the following: (GS)ₓ, wherein X=1-15. In some embodiments, the GS linker comprises the following: (G_{y}S)ₓ; x=1-15, y=1-10.

In some embodiments, the ubiquitin pathway moiety and the binding peptide are not separated by a linker.

In some embodiments, the ubiquitin pathway moiety is an E3 adaptor protein.

In some embodiments, the E3 adaptor protein is engineered to replace its substrate recognition domain with the binding peptide.

In some embodiments, the E3 adaptor protein is selected from SPOP, CHIP, CRBN, VHL, XIAP, MDM2, cereblon and cIAP. Accordingly, in some embodiments, the E3 adaptor protein is SPOP. In some embodiments, the E3 adaptor protein is CHIP. In some embodiments, the E3 adaptor protein is VHL. In some embodiments, the E3 adaptor protein is XIAP. In some embodiments, the E3 adaptor protein is MDM2. In some embodiments, the E3 adaptor protein is cereblon. In some embodiments, the E3 adaptor protein is cIAP.

In some embodiments, the ubiquitin pathway moiety is an antibody that specifically binds an E3 adaptor protein or E3 ligase. In some embodiments, the antibody that specifically binds an E3 adaptor protein is SPOP, CHIP, CRBN, VHL, XIAP, MDM2 or cIAP. In some embodiments, the antibody that specifically binds an E3 adaptor protein is SPOP. In some embodiments, the antibody that specifically binds an E3 adaptor protein is CHIP. In some embodiments, the antibody that specifically binds an E3 adaptor protein is CRBN. In some embodiments, the antibody that specifically binds an E3 adaptor protein is VHL. In some embodiments, the antibody that specifically binds an E3 adaptor protein is XIAP. In some embodiments, the antibody that specifically binds an E3 adaptor protein is MDM2. In some embodiments, the antibody that specifically binds an E3 adaptor protein is cIAP.

In some embodiments, the binding peptide is an antibody or antibody fragment. In some embodiments, the binding peptide is an antibody or antibody fragment that specifically binds to the target protein.

In some embodiments, the binding peptide is a protein that binds to or forms a complex with the target protein. In some embodiments, the protein that binds to or forms a complex with the target protein of interest is endogenous to a target cell. In some embodiment, the target protein is aberrantly expressed in a target cell. In some embodiments, the target protein is an intracellular protein. In some embodiments, the target protein is a nuclear protein. In some embodiments, the target protein is an enzyme. In some embodiments, the target protein is a protein involved in cell signaling. In some embodiments, the target protein is protein involved in cell division. In some embodiments, the target protein is protein involved in metabolism. In some embodiments, the target protein is protein involved in inflammatory response.

In one aspect, the present invention provides, among other things, a messenger RNA (mRNA) that encodes at least two binding peptides, wherein a first binding peptide binds a ubiquitin pathway moiety and a second binding peptide binds a target protein, and wherein the mRNA is encapsulated within a lipid nanoparticle. In some embodiments, a single mRNA encodes at least two binding peptides, wherein a first binding peptide binds a ubiquitin pathway moiety and a second binding peptide binds a target protein, and wherein the mRNA is encapsulated within a lipid nanoparticle. In some embodiments, at least two mRNAs are provided comprising a first mRNA which encodes a first binding peptide, and a second mRNA which encodes a second binding peptide. In some embodiments, the first mRNA and the second mRNA are encapsulated in a separate lipid nanoparticle. In some embodiments, the first and the second mRNA are encapsulated in a single lipid nanoparticle. In some embodiments, the binding peptides encoded by the first mRNA and the second mRNA bind to each other creating a bound fusion-like moiety.

In some embodiments, the first binding peptide and the second binding peptide are separated by a linker.

In some embodiments, the linker is a GS linker.

In some embodiments, the first binding peptide and the second binding peptide are not separated by a linker.

In some embodiments, the ubiquitin pathway moiety is a ubiquitin pathway protein.

In some embodiments, the ubiquitin pathway moiety is an E3 adaptor protein.

In some embodiments, the E3 adaptor protein is selected from SPOP, CHIP, CRBN, VHL, XIAP, MDM2 and cIAP.

In some embodiments, the first binding peptide is an antibody or antibody fragment.

In some embodiments, the second binding peptide is an antibody or antibody fragment.

In some embodiments, the antibody or antibody fragment is a nanobody, Fab, Fab′, Fab′2, F(ab′)2, Fd, Fv, Feb, scFv, or SMIP. In some embodiments, the antibody or antibody fragment binds to an E3 ligase adaptor protein. In some embodiments, the antibody or antibody fragment binds to SPOP, CHIP, CRBN, VHL, XIAP, MDM2, cereblon and/or cIAP. Accordingly, in some embodiments, the construct encodes an antibody or antibody fragment that binds to SPOP. In some embodiments, the construct encodes an antibody or antibody fragment that binds to CHIP. In some embodiments, the construct encodes an antibody or antibody fragment that binds to CRBN. In some embodiments, the construct encodes an antibody or antibody fragment that binds to VHL. In some embodiments, the construct encodes an antibody or antibody fragment that binds to XIAP. In some embodiments, the construct encodes an antibody or antibody fragment that binds to MDM2. In some embodiments, the construct encodes an antibody or antibody fragment that binds to cereblon. In some embodiments, the construct encodes an antibody or antibody fragment that binds to cIAP.

In some embodiments, the mRNA further encodes a signal peptide.

In some embodiments, the signal peptide is a nuclear localization sequence.

In some embodiments, the signal peptide is an endoplasmic reticulum (ER) signal sequence.

In some embodiments, the signal peptide is an endoplasmic reticulum (ER) retention sequence.

In some embodiments, the signal peptide is a cell secretory sequence.

In some embodiments, the lipid nanoparticle comprises one or more cationic lipids, one or more non-cationic lipids, one or more cholesterol-based lipids and one or more PEG-modified lipids.

In some embodiments, the one or more cationic lipids are selected from the group consisting of cKK-E12, OF-02, C12-200, MC3, DLinDMA, DLinkC2DMA, ICE (Imidazol-based), HGT5000, HGT5001, HGT4003, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, 3-(4-(bis(2-hydroxydodecyl)amino)butyl)-6-(4-((2-hydroxydodecyl)(2-hydroxyundecyl)amino)butyl)-1,4-dioxane-2,5-dione (Target 23), 3-(5-(bis(2-hydroxydodecyl)amino)pentan-2-yl)-6-(5-((2-hydroxydodecyl)(2-hydroxyundecyl)amino)pentan-2-yl)-1,4-dioxane-2,5-dione (Target 24), and combinations thereof.

In some embodiments, the one or more cationic lipids comprise cKK-E12.

In some embodiments, the target protein comprises a phosphorylated version of the target protein, a non-phosphorylated version of the target protein, a lipidated version of the target protein, a non-lipidated version of the target protein, a pro-peptide version of the target protein, a glycosylated version of the target protein, an unglycosylated version of the target protein, an oxidized version of the target protein, an unoxidized version of the target protein, a carbonylated version of the target protein, a non-carbonylated version of the target protein, a formylated version of the target protein, a non-formylated version of the target protein, an acylated version of the target protein, a non-acylated version of the target protein, an alkylated version of the target protein, a non-alkylated version of the target protein, a sulfonated version of the target protein, a non-sulfonated version of the target protein, an s-nitrosylated version of the target protein, a non-s-nitrosylated version of the target protein, a glutathione addition version of the target protein, a non-glutathione addition version of the target protein, an adenylated version of the target protein, a non-adenylated version of the target protein, or an ATP or ADP bound version of the protein.

In some embodiments, the target protein is bound to a receptor.

In one aspect, a pharmaceutical composition comprising the mRNA of any one of the embodiments described herein is provided.

In one aspect, a method of inducing protein degradation comprising administering the mRNA as described in any one of the embodiments described herein is provided.

In some embodiments, the mRNA is administered intravenously, intradermally, subcutaneously, intrathecally, orally, or by inhalation or nebulization.

In one aspect, a cell comprising the mRNA as described in any one of the embodiments described herein is provided.

In one aspect, a method of treating a subject suffering from a disease or disorder associated with aberrant protein expression, comprising administering to the subject in need thereof an mRNA as described herein, wherein administration of the mRNA results in selective degradation of the aberrantly expressed protein.

In some embodiments, the disease or disorder is prion-based disease. In some embodiments, the disease or disorder is polycystic kidney disease. In some embodiments, the disease or disorder is Pelizaeus-Mezbacher disease. In some embodiments, the disease or disorder is an inflammatory disease. In some embodiments, the disease or disorder is cancer.

### BRIEF DESCRIPTION OF FIGURES

The drawings are for illustration purposes, not for limitation.

FIG. 1A is a schematic representation of the mRNA constructs comprising sequences encoding vhhGFP4, E3 ligase, and a FLAG tag. Optionally, constructs comprises a sequence encoding ER signal peptide, ER retention signal and/or a linker as shown as “A”.

FIG. 1B shows mRNA construct subcellular localization and design for constructs A and E.

FIG. 2A is an image of untreated, GFP-expressing HeLa cells. GFP is shown as indicated in the upper left panel, nuclear DNA staining is shown in the upper right panel, and FLAG, which indicates E3-ubiquitin ligase expression, is shown in the lower left panel. The lower right panel is a merge image. FIG. 2B is a merge image of the GFP and FLAG signals.

FIG. 3A is an image of GFP-expressing HeLa cells after 24 hours of transfection with mRNA Construct A (as depicted in FIG. 1A and FIG. 1B). GFP is shown in the upper left panel, nuclear DNA is shown in the upper right panel, and FLAG, which indicates E3-ubiquitin ligase expression, is shown in the lower left panel. A merged image is presented in the lower right panel. FIG. 3B is a magnified merge image of GFP and FLAG signals. The arrows indicate exemplary cells which shows reduced or absent GFP signal in cells that contain the vector construct (i.e., those that have the SPOP E3-ubiquitin ligase).

FIG. 4A is an image of GFP-expressing HeLa cells after 24 hours of transfection with mRNA Construct C, which contains an ER signal peptide and an ER retention signal (indicated in FIG. 1A and FIG. 1B). GFP is shown in the upper left panel, DNA is shown in the upper right panel, and FLAG, which indicates E3-ubiquitin ligase expression, is shown in the lower left panel. A merge image is presented in the lower right panel. FIG. 4B is a magnified merge image of GFP and FLAG signals. Dashed arrows indicate exemplary cells which were transfected with the vector (as indicated by the FLAG immuno staining) and had reduced amounts of GFP present. Solid arrows indicate exemplary cells which expressed E3-ubiquitin ligase, with reduced or absent GFP signal.

FIG. 5A is an image of GFP-expressing HeLa cells after 24 hours of transfection with mRNA Construct D (as indicated in FIG. 1A and FIG. 1B). GFP is shown in upper left panel, nuclear DNA is shown in upper right panel, and FLAG, which indicates E3-ubiquitin ligase expression, is shown in the lower left panel. A merge image is presented in the lower right panel. FIG. 5B is a magnified merge image of GFP and FLAG signals. Dashed arrows indicate exemplary cells which expressed both GFP and E3-ubiquitin ligase. Solid arrows indicate exemplary cells which expressed E3-ubiquitin ligase, with reduced or absent GFP signal.

FIG. 6A is an image of GFP-expressing HeLa cells after 24 hours of transfection with mRNA Construct E. GFP is shown in the upper left panel, nuclear DNA is shown in the upper right panel, and FLAG, which indicates E3-ubiquitin ligase expression, is shown in the lower right panel. A merge image is presented in the lower right panel. FIG. 6B is a magnified merge image of GFP and FLAG signals. Dashed arrows indicate exemplary cells which expressed both GFP and E3-ubiquitin ligase. Solid arrows indicate exemplary cells which expressed E3-ubiquitin ligase, with reduced or absent GFP signal.

FIG. 7A is an image of GFP-expressing HeLa cells after 24 hours of transfection with mRNA Construct F (as described in FIG. 1A and FIG. 1B). GFP is shown in the upper left panel, nuclear DNA is shown in the upper right panel, and FLAG, which indicates E3-ubiquitin ligase expression, is shown in the lower left panel. FIG. 7B is a magnified merge image of GFP and FLAG signals. Dashed arrows indicate exemplary cells which expressed both GFP and E3-ubiquitin ligase. Solid arrows indicate exemplary cells which expressed E3-ubiquitin ligase, with reduced or absent GFP signal.

FIG. 8A is a series of images of HEK293 cells after 6 hours of transfection. In the upper left panel, the untreated HEK293 cells (Sample 1 as described in Table 2) shows signal for only nuclear DNA. In the upper right panel (Sample 2), cells transfected by GFP mRNA are shown, which shows signals for nuclear DNA and GFP. In the lower left panel (Sample 3), cells transfected by construct A (as described in FIG. 1A and FIG. 1B) are shown, which shows staining for nuclear DNA and FLAG, indicating E3-ubiquitin ligase localization as nuclear speckles. In the lower right panel (Sample 4), cells transfected by construct E (as described in FIG. 1A and FIG. 1B) are shown, which shows signals for nuclear DNA and FLAG, indicating E3-ubiquitin ligase localization in the cytoplasm. FIG. 8B is a series of images of HEK293 cells after 24 hours of transfection. In the upper left panel, the untreated HEK293 cells (Sample 7 as described in Table 2) shows signal for only nuclear DNA. In the upper right panel (Sample 8), cells transfected by GFP mRNA are shown, which shows signal for nuclear DNA and GFP. In the lower left panel (Sample 9), cells transfected by construct A (as described in FIG. 1A and FIG. 1B) are shown, which shows signal for nuclear DNA and FLAG, indicating E3-ubiquitin ligase localization as nuclear speckles. In the lower right panel (Sample 10), cells transfected by construct E (as described in FIG. 1A and FIG. 1B) are shown, which shows signal for nuclear DNA and FLAG, indicating E3-ubiquitin ligase localization in the cytoplasm.

FIG. 9A is a series of images of HEK293 cells after 6 hours of transfection with construct A and GFP mRNA (Sample 5 as shown in Table 2). GFP signal is shown in the left panel. Right panel shows a merge image of GFP and FLAG signals. Solid arrows indicate exemplary cells which expressed E3-ubiquitin ligase, with reduced or absent GFP signal. FIG. 9B is a series of images of HEK293 cells after 24 hours of transfection with construct A and GFP mRNA (Sample 11 as shown in Table 2). GFP signal is shown in the left panel. Right panel shows a merge image of GFP and FLAG signals. Solid arrows indicate exemplary cells which expressed E3-ubiquitin ligase, with reduced or absent GFP signal.

FIG. 10A is a series of images of HEK293 cells after 6 hours of transfection with construct E and GFP mRNA (Sample 6 as shown in Table 2). GFP signal is shown in the left panel. Right panel shows a merge image of GFP and FLAG signals. Solid arrows indicate exemplary cells which expressed E3-ubiquitin ligase, with reduced or absent GFP signal. FIG. 10B is a series of images of HEK293 cells after 24 hours of transfection with construct E and GFP mRNA (Sample 12 as shown in Table 2). GFP signal is shown in the left panel. Right panel shows a merge image of GFP and FLAG signals. Solid arrows indicate exemplary cells which expressed E3-ubiquitin ligase, with reduced or absent GFP signal.

FIG. 11 is a series of images of H2B-tagged GFP-expressing HeLa cells after 24 hours of transfection with construct A. DAPI signal, which indicates nuclear DNA is shown in the upper left panel, GFP is shown in the upper right panel, and FLAG, which indicates E3-ubiquitin ligase expression, is shown in the lower left panel. Lower right panel a merge image of GFP and FLAG signals.

FIG. 12 is a series of images of H2B-tagged GFP-expressing HeLa cells after 24 hours of transfection with construct E. DAPI signal, which indicates nuclear DNA is shown in the upper left panel, GFP is shown in the upper right panel, and FLAG, which indicates E3-ubiquitin ligase expression, is shown in the lower left panel. Lower right panel a merge image of GFP and FLAG signals.

FIG. 13A-D depict a series of graphs and Western blots that show a dose-response effect of construct E. FIG. 13A show san exemplary graph depicting a dose-response effect of E3-ubiqtion ligase encoded by construct E on proteolysis of GFP. HeLa cells that do not endogenously express GFP were co-transfected with GFP mRNA and construct E at various concentrations. ELISA was used to determine the concentration of GFP 24 hours after co-transfection. FIG. 13B shows the percent knockdown via ELISA of GFP in HeLA cells after treatment with Construct E and GFP mRNA. FIG. 13C depicts a FLAG Western Blot. FIG. 13D depicts both a GFP Western Blot and a graph that shows GFP expression was reduced in a concentration dependent manner.

FIG. 14 is an exemplary graph depicting a time-course study of GFP degradation induced by E3-ubiqtion ligase encoded by construct E. HeLa cells that do not endogenously express GFP were co-transfected with GFP mRNA and construct E. ELISA was used to determine the concentration of GFP at various time points from 0 to 34 hours post-transfection.

FIG. 15 is an exemplary graph depicting a time-course study of GFP degradation induced by E3-ubiqtion ligase encoded by construct A. HeLa cells that stably express H2B-GFP in the nucleus were transfected with construct A. ELISA was used to determine the concentration of GFP at various time points from 0 to 72 hours post-transfection.

FIG. 16 is an exemplary schematic depicting a study design of in vitro cell-free translation system. Cytoplasmic extracts are prepared from HeLa cells. Cytoplasmic extracts, which contain functional translation system, are supplemented with mRNA encoding a target protein (e.g. GFP or A1AT) or a recombinant protein, in addition to mRNAs encoding E3-ubiquitin ligase. At various time points, samples are taken to quantify the amount of the target protein by ELISA, Western blot, or qPCR.

FIG. 17A is an exemplary graph depicting a time-course study of GFP degradation induced by E3-ubiqtion ligase encoded by construct E in the cell-free translation system (CFTS). Cytoplasmic extracts were supplemented with GFP mRNA (5 pmol) and construct E at various ratios of GFP mRNA:Construct E. As negative controls, a sample was supplemented with only with GFP mRNA, and another sample was not supplemented with any mRNA. The amount of GFP protein was quantified at various time points by ELISA. FIG. 17B is a graph that shows a time course study of recombinant GFP degradation induced by E3-ubiqtion ligase encoded by construct E in the cell-free translation system (CFTS). FIG. 17C is a schematic of Construct G, comprising the E3 ligase cereblon. FIG. 17D is a graph that shows anti-GFP concentration response using Construct G in a cell-free translation system (CFTS). FIG. 17E is a graph that shows percentage GFP at 1 hour, 2, hours, and 3 hours of contact with Construct G at 2× or 6× concentration. FIG. 17F is a schematic showing various bioPROTAC designs that include the E3 ligase cereblon. The bioPROTAC designs include Construct M which encodes an anti-PNPLA3 scFv, and construct N which includes ABHD5 a PNPLA3 protein binder. FIG. 17G is a graph that shows data obtained from ELISA assays that show a concentration dependent decrease in the amount of PNPLA3 with increasing concentration of bioPROTAC construct M.

FIG. 18A is a schematic representation of mRNA constructs comprising sequences encoding vhhGFP4, SPOP E3-ligase, and a FLAG tag. SPOP E3-ligase contains a nucleus localization signal (NLS). Various linker lengths were introduced between vhhGFP4 and SPOP to examine the effect of linker length on GFP proteolysis. FIG. 18B is an exemplary graph depicting a time-course study of GFP degradation induced by E3-ubiqtion ligase encoded by construct A with various linker lengths (Constructs A1-A5; Table 4) in the cell-free translation system. Cytoplasmic extracts were supplemented with GFP mRNA and variants of Construct A. As a negative control, a sample was supplemented with only with GFP mRNA. The amount of GFP protein was quantified at various time points by ELISA.

FIG. 19 is a schematic representation of the mRNA constructs comprising sequences encoding scFv4B12 that specifically targets A1AT, E3 ligase (hVHL or CHIP), and a FLAG tag. Optionally, constructs comprises a sequence encoding ER signal peptide, ER retention signal and/or a linker as shown as “A”.

FIG. 20A is an exemplary graph depicting a dose-response effect of E3-ubiqtion ligase encoded by construct E on proteolysis of A1AT. HeLa cells that do not endogenously express A1AT were co-transfected with A1AT plasmid and constructs shown in FIG. 19 at various concentrations. ELISA was used to determine the concentration of A1AT 24 hours after co-transfection. FIG. 20B is an exemplary graph depicting a dose-response effect of E3-ubiqtion ligase encoded by construct E on proteolysis of A1AT in in vitro cell-free translation system. Cytoplasmic extracts were supplemented with A1AT mRNA at 4 pmol and constructs shown in FIG. 19, at various ratios of A1AT mRNA: Construct. As a negative control, a sample was supplemented with only with A1AT mRNA. The amount of A1AT protein was quantified at various time points by ELISA

FIGS. 21A and B depict a schematic, a graph and Western blots that show a dose response effect of construct G. FIG. 21 A shows a schematic of construct G and a graph that shows the percentage of GFP Knockdown in HeLA cells after treatment with Construct G bioPROTAC RNA and GFP mRNA. FIG. 21B shows GFP Western Blots from studies using Construct G and an associated graphical representation of same. FIG. 21C shows a FACS plot of HeLA cells transfected with different ratios of Construct G and GFP RNA (1:1, 4:1; and 10:1). FIG. 21D is a bar graph that shows GFP expression in the 1:1 ratio condition of Construct G and GFP RNA, with or without the proteasomal inhibitor MG132.

FIG. 22A is a graph that shows results of a GFP ELISA from HeLA cells treated with Construct G bioPROTAC RNA with or without 5 uM proteome inhibitor, MG-132. FIG. 22B depicts a GFP Western Blot with and without proteasome inhibitor MG-132. FIG. 22B also shows a graph that corresponds to the GFP Western Blot results.

FIG. 23A is a schematic that shows the designs of various bioPROTAC designs, including bi-specific anti-cereblon bioPROTACs. FIG. 23B is a schematic that illustrates binding of the bioPROTAC to cereblon (CRBN) in an E3 ligase complex. FIG. 23C is a graph that shows the percentage Knockdown in HeLa cells co-transfected with GFP RNA and bioPRTOAC RNA at various concentrations.

FIG. 24A is a schematic that shows the designs of various bioPROTACs used to assess the duration of expression of in vivo administered bioPROTACs. FIG. 24B is a graph that shows liver GFP expression (μg GFP/mg protein) at 6 hours and at 24 hours post-administration.

### DEFINITIONS

In order for the present invention to be more readily understood, certain terms are first defined below. Additional definitions for the following terms and other terms are set forth throughout the specification.

PROTAC: PROTAC, a proteolysis targeting chimera, is a heterofunctional small molecule composed of two active domains and optionally a linker capable of removing specific unwanted proteins. Rather than acting as a conventional enzyme inhibitor, a PROTAC works by inducing selective intra cellular proteolysis. PROTACs generally consist of two covalently linked protein-binding molecules: one capable of engaging an E3 ubiquitin ligase, and another that binds to a target protein meant for degradation. Recruitment of the E3 ligase to the target protein results in ubiquitination and subsequent degradation of the target protein by the proteasome. PROTACs need only to bind their targets with high selectivity, rather than inhibit the target protein's enzymatic activity. The PROTAC technology can be applied in drug discovery using various E3 ligases, including for example, SPOP, CHIP, pVHL, MDM2, beta-TrCP1, cereblon, and c-IAP1.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

Encapsulation: As used herein, the term “encapsulation,” or grammatical equivalent, refers to the process of confining an individual mRNA molecule within a nanoparticle.

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalent, are used inter-changeably.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Local distribution or delivery: As used herein, the terms “local distribution,” “local delivery,” or grammatical equivalent, refer to tissue specific delivery or distribution. Typically, local distribution or delivery requires a protein (e.g., enzyme) encoded by mRNAs be translated and expressed intracellularly or with limited secretion that avoids entering the patient's circulation system.

Messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Systemic distribution or delivery: As used herein, the terms “systemic distribution,” “systemic delivery,” or grammatical equivalent, refer to a delivery or distribution mechanism or approach that affect the entire body or an entire organism.

Typically, systemic distribution or delivery is accomplished via body's circulation system, e.g., blood stream. Compared to the definition of “local distribution or delivery.”

Target cell: As used herein, the term “target cell” refers to any cell that is affected by a disease to be treated. In some embodiments, a target cell displays a disease-associated pathology, symptom, or feature.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides an mRNA-based composition and method for the selective degradation of a target protein of interest. The mRNA composition described herein encodes a ubiquitin pathway moiety that is coupled (directly or indirectly via a linker) with a binding peptide of interest. Upon expression of the ubiquitin pathway moiety and the binding peptide, the binding protein binds to the protein of interest and the ubiquitin pathway moiety causes ubiquitination and selective degradation of the protein of interest. Accordingly, one of the uses of the mRNA described herein is the selective, rapid degradation of a target protein of interest.

In particular embodiments, an mRNA-based PROTAC composition is provided. Also provided are methods of treating disease associated with aberrant expression of a target protein using mRNA encoding a ubiquitin targeting moiety fused with a binding protein specific for the target protein. Such compositions are described herein, and in some embodiments, the mRNA is delivered to a subject in need thereof by way of a lipid nanoparticle delivery system.

Various aspects of the invention are described in detail in the following sections. The use of sections is not meant to limit the invention. Each section can apply to any aspect of the invention. In this application, the use of “or” means “and/or” unless stated otherwise.

mRNA Encoding Ubiquitin Pathway Moiety and Binding Protein

According to the present invention, a ubiquitin pathway moiety can be any suitable structure that recognizes and binds to a ubiquitin pathway protein. In general, a ubiquitin pathway protein can be any entity or complex that is capable of catalyzing or causing to catalyze the transfer of a ubiquitin or ubiquitin-like modifying polypeptide, e.g., Nedd8, APG12 or ISG15/UCRP to another protein, a protein of interest. In one embodiment, a ubiquitin pathway protein is a ubiquitin protein ligase or E3 adaptor protein or E3-ubiquitin ligase. There are at least 600 E3 ligases that are encoded by the human genome (see Lim et al., bioRxiv preprint, “bioPROTACs as versatile modulators of intracellular therapeutic targets: Application to proliferating cell nuclear antigen (PCNA),” dx.doi.org/10.1101/728071, the contents of which are incorporated by reference herein in its entirety). Any of the available E3 ligases or adaptor proteins can be used in the invention described herein. Of these E3 ligases, the most commonly used ones include, for example, CRBN, VHL, MDM2 and cIAP. In some embodiments, the mRNA of the invention encodes an E3 ligase selected from SPOP, CHIP, CRBN, VHL, MDM2 and cIAP.

In some aspects, an mRNA that encodes at least two binding peptides is provided, wherein a first binding peptide binds a ubiquitin pathway moiety and a second binding peptide binds a target protein, and wherein the mRNA is encapsulated within a lipid nanoparticle.

In another embodiment, a ubiquitin pathway moiety can be a protein that is involved in or a component of a ubiquitin-like pathway, which transfers ubiquitin-like modifying polypeptides, e.g., SUMO, Nedd8, APG12 or ISG15/UCRP. Components of a ubiquitin-like pathway are usually homologues of a ubiquitin pathway. For example, the ubiquitin-like pathway for SUMO can include a homologue of a ubiquitin protein activating enzyme or E1 protein, ubiquitin protein conjugating enzyme or E2 protein and ubiquitin ligase or E3 protein.

A ubiquitin pathway protein can be expressed in a tissue specific or regulated manner. For example, VACM-1 receptor (aka CUL-5), and F-box protein, NFB42 are expressed in a tissue specific manner. In one embodiment, a ubiquitin pathway protein can be an RING-based or HECT-based ubiquitin ligase.

According to one embodiment of the present invention, a ubiquitin pathway moiety of the present invention can be any suitable ligand to a ubiquitin pathway protein, e.g., ubiquitin protein ligase or E3 adaptor protein or homologues thereof. In another embodiment, a ubiquitin pathway moiety of the present invention can be any ubiquitin pathway protein binding peptide, domain or region of a ligand to a ubiquitin pathway protein. In still another embodiment, a ubiquitin pathway protein binding moiety of the present invention can recognize and bind to a ubiquitin pathway protein in a regulated manner.

In some embodiments, E3 adaptor protein can be used in its native form. In some embodiments, E3 adaptor protein can be engineered to replace its substrate recognition domain with the binding peptide. In some embodiments, E3 adaptor protein can be selected from SPOP, CHIP, CRBN, VHL, XIAP, MDM2 and cIAP. In one embodiment, E3 adaptor protein is SPOP. In another examples, E3 adaptor protein is VHL.

According to the present invention, a targeting moiety or binding peptide is any structure that recognizes and binds to a target protein. For example, a binding peptide maybe an endogenous protein that binds to or forms a complex with a target protein. Alternatively, a binding peptide may be an antibody or antibody fragment that specifically binds the target protein. A target protein can be any protein that one desires to regulate its level or activity, e.g., to alter the activity through ubiquitin-dependent proteolysis or through attachment of ubiquitin or ubiquitin-like modifying polypeptide to lysine residues that are important for the protein's activity or structure. Typically, the target protein is aberrantly expressed in a target cell. For example, a target protein can be a protein involved in cell cycle (e.g., a cyclin-dependent kinase), signal transduction (e.g., a receptor tyrosine kinase or GTPase, or the like), cell differentiation, cell dedifferentiation, cell growth, production of cytokines or other biological modifiers, production of regulatory or functional proteins (e.g., a transcription factor), pro-inflammatory signaling, or the glucose regulation pathway. In one embodiment, a target protein can be a protein that is not known to be ubiquitinated or not known to be a substrate for any ubiquitin pathway protein.

In another embodiment, a target protein is a disease related protein, e.g., a protein for which changes in its function or activity cause disease, or whose function is considered important to the propagation of the disease state. The target protein may be either stable or unstable, e.g., androgen receptor, estrogen receptor, myc, cyclin B, Ras, or cyclin E.

In some embodiments, a target protein is A1AT. In some embodiments, a target protein is PNPLA3. In some embodiments, a target protein is a protein that forms aggregates. In some embodiments, a target protein is tau. In some embodiments, a target protein is β-amyloid. In some embodiments, a target protein is α-synuclein. In some embodiments, a target protein is prion. In some embodiments, a target protein is TDP-43, fused in sarcoma protein, cystain C, Notch3, GFAP, PLP, seipin, transthyretin, serpins, amyloid A protein, IAPP, apolipoprotein, gelsolin, lysozyme, fibrinogen, insulin, or hemoglobin.

### Selective Degradation of Target Protein

The compositions and methods described herein are useful for selective targeting of a protein of interest (“target protein”) for degradation. The selective targeting of a target protein includes selective targeting of a protein that has a specific kind of post-translational modification.

For example, in some embodiments, the compositions and methods described herein are used to target a protein for degradation when the target protein is phosphorylated. In some embodiments, the compositions and methods described herein are used to target a protein for degradation when the target protein is unphosphorylated. In some embodiments, a lipidated version of the target protein.

In some embodiments, the compositions and methods described herein are used to target a protein for degradation when the target protein is a non-lipidated version of the target protein.

In some embodiments, the compositions and methods described herein are used to target a protein for degradation when the target protein is a pro-peptide version of the target protein.

In some embodiments, the compositions and methods described herein are used to target a protein for degradation when the target protein is a glycosylated version of the target protein.

In some embodiments, the compositions and methods described herein are used to target a protein for degradation when the target protein is an unglycosylated version of the target protein.

In some embodiments, the compositions and methods described herein are used to target a protein for degradation when the target protein is an oxidized version of the target protein,

In some embodiments, the compositions and methods described herein are used to target a protein for degradation when the target protein is an unoxidized version of the target protein.

In some embodiments, the compositions and methods described herein are used to target a protein for degradation when the target protein is a carbonylated version of the target protein.

In some embodiments, the compositions and methods described herein are used to target a protein for degradation when the target protein is a non-carbonylated version of the target protein,

In some embodiments, the compositions and methods described herein are used to target a protein for degradation when the target protein is a formylated version of the target protein.

In some embodiments, the compositions and methods described herein are used to target a protein for degradation when the target protein is a non-formylated version of the target protein.

In some embodiments, the compositions and methods described herein are used to target a protein for degradation when the target protein is an acylated version of the target protein.

In some embodiments, the compositions and methods described herein are used to target a protein for degradation when the target protein is a non-acylated version of the target protein.

In some embodiments, the compositions and methods described herein are used to target a protein for degradation when the target protein is an alkylated version of the target protein,

In some embodiments, the compositions and methods described herein are used to target a protein for degradation when the target protein is a non-alkylated version of the target protein.

In some embodiments, the compositions and methods described herein are used to target a protein for degradation when the target protein is a sulfonated version of the target protein,

In some embodiments, the compositions and methods described herein are used to target a protein for degradation when the target protein is a non-sulfonated version of the target protein.

In some embodiments, the compositions and methods described herein are used to target a protein for degradation when the target protein is an s-nitrosylated version of the target protein.

In some embodiments, the compositions and methods described herein are used to target a protein for degradation when the target protein is a non-s-nitrosylated version of the target protein.

In some embodiments, the compositions and methods described herein are used to target a protein for degradation when the target protein is a glutathione addition version of the target protein.

In some embodiments, the compositions and methods described herein are used to target a protein for degradation when the target protein is a non-glutathione addition version of the target protein.

In some embodiments, the compositions and methods described herein are used to target a protein for degradation when the target protein is an adenylated version of the target protein.

In some embodiments, the compositions and methods described herein are used to target a protein for degradation when the target protein is a non-adenylated version of the target protein.

In some embodiments, the compositions and methods described herein are used to target a protein for degradation when the target protein is an ATP or ADP bound version of the protein.

In some embodiments, the compositions and methods described herein are used to target a protein for degradation when the target protein has one or more post-translational modifications. For example, the target protein can have one or more of the following post-translational modifications: acetylation, amidation, deamidation, prenylation (such as farnesylation or geranylation), formylation, glycosylation, hydroxylation, methylation, myristoylation, phosphorylation, sialylation, polysialylation, SUMOylation, NEDDylation, ribosylation, sulphation, or any combinations thereof.

In some embodiments, the compositions and methods described herein are used to selectively degrade a target protein that is bound to another protein. For example, the compositions and methods described herein can be used to selectively degrade a target protein that is bound to a receptor. In some embodiments, the compositions and methods described herein can be used to selectively degrade a target protein that is not bound to a receptor.

In some embodiments, the compositions and methods described herein are used to selectively degrade a target protein which has a long half-life. Many such long half-life proteins are known in the art and include, for example, cell structure proteins.

### Binding Peptide

According to the present invention, a binding peptide or targeting moiety is any structure that recognizes and binds to a target protein or protein of interest (POI), e.g. a protein (e.g., an intracellular protein) that is aberrantly expressed in a target cell of interest. This can be for example a ligand, an antibody or antibody fragment. According to the present invention, the ubiquitin pathway protein moiety is coupled, e.g., covalently by any suitable means to the targeting moiety or binding peptide of interest. In some embodiments, the composition of the present invention includes an mRNA that encodes a chimeric fusion protein comprising a ubiquitin pathway moiety (e.g., an E3 adaptor protein or E3 ligase) fused with a binding protein that targets a protein of interest (e.g., an antibody). In other embodiments, the composition of the present invention includes an mRNA that encodes a chimeric fusion protein comprising a ubiquitin pathway moiety (e.g., an antibody that specifically binds an E3 adaptor protein or E3 ligase) fused with a binding protein that targets a protein of interest (e.g., an antibody). Upon expression of the chimeric fusion protein, the binding protein binds to the protein of interest and the ubiquitin pathway moiety causes ubiquitination and selective degradation of the protein of interest.

In some embodiments, the binding peptide can be a member of a molecular library. A molecular library can be any collection of molecules, including without limitation, a combinatorial library, a small molecule library, a receptor library, and a ligand library.

A binding peptide can be a peptide, an antibody, or an antibody-mimetic which allows for binding to a vast diversity of target proteins, e.g. a protein (e.g., an intracellular protein) that is aberrantly expressed in a target cell of interest. In some embodiments, a binding protein is an antibody, an antibody fragment or an antibody domain.

In particular embodiments, a binding peptide can be an endogenous protein, or a fragment thereof, that specifically binds to a target protein of interest. For example, the endogenous protein, or fragment thereof, may form a complex with the target protein of interest. Accordingly, the composition of the present invention includes an mRNA that encodes a chimeric fusion protein comprising a ubiquitin pathway moiety (e.g., an E3 adaptor protein or E3 ligase such as an endogenous E3 adaptor protein or E3 ligase) fused with an endogenous protein that specifically binds to or forms a complex with a target protein of interest. In particular embodiments, the mRNA encodes a chimeric fusion protein comprising an endogenous ubiquitin pathway moiety that is engineered to replace its substrate recognition domain with an endogenous protein that binds to or forms a complex with a target protein of interest. Such fusion proteins comprising or consisting of components endogenously expressed in the human body (i.e., peptides or proteins that are normally express in the human body) may be particularly advantageous because they are unlikely to elicit any immunogenic reaction that may be encountered if the fusion protein encodes peptides or proteins that are exogenous to the human body (i.e., peptides or proteins that are not normally expressed in the human body and therefore may elicit an immune response if expressed in a target cell of interest).

In other embodiments, a binding protein can be an antibody that specifically binds to a target protein of interest, e.g. a protein (e.g., an intracellular protein) that is aberrantly expressed in a target cell of interest. The versatility of antibodies in specifically binding proteins of interest and the diversity of antibody formats make their use in the fusion proteins of the invention particularly attractive. Moreover, a wide variety of highly specific antibodies to target proteins implicated in disease mechanisms are known, so that the creation of fusion proteins with a particular specificity to a target protein of interest is relatively straightforward and inexpensive.

Accordingly, in some embodiments, the composition of the present invention includes an mRNA that encodes a chimeric fusion protein comprising a ubiquitin pathway moiety (e.g., an E3 adaptor protein or E3 ligase) fused with an antibody that specifically binds to a target protein of interest.

In some embodiments, the antibody is a single-domain antibody (sdAb), e.g., a nanobody, Fab, Fab′, Fab′2, F(ab′)2, Fd, Fv, Feb, scFv, or SMIP. Accordingly, in some embodiments, the antibody is single-domain antibody (sdAb), e.g., a nanobody. In some embodiments, the antibody is a Fab. In some embodiments, the antibody is a Fab′. In some embodiments, the antibody is a Fab′2. In some embodiments, the antibody is a Fab′2. In some embodiments, the antibody is a Fd. In some embodiments, the antibody is a Fv. In some embodiments, the antibody is a Feb. In some embodiments, the antibody is a scFv. In some embodiments, the antibody is a SMIP.

As is recognized in the art, a nanobody is a single-domain antibody (sdAb) that has a single monomeric variable antibody domain. In some embodiments, a nanobody can be a VHH fragment or a VNAR fragments. The nanobody, a nanobody can be an anti-GFP-nanobody, vhhGFP4. sdAbs that specifically bind a target protein of interest are particularly suitable for use in the compositions of the invention because they are relatively small in size and therefore can diffuse more easily to subcellular locations. Accordingly, in some embodiments, the composition of the present invention includes an mRNA that encodes a chimeric fusion protein comprising a ubiquitin pathway moiety (e.g., an E3 adaptor protein or E3 ligase) fused with an sdAb that specifically binds to a target protein of interest. In other embodiments, the composition of the present invention includes an mRNA that encodes a chimeric fusion protein comprising an sdAb that specifically binds an E3 adaptor protein or E3 ligase fused with an sdAb that specifically binds to a target protein of interest

A target protein can be any protein that one desires to regulate its level or activity, e.g., to alter the activity through ubiquitin-dependent proteolysis or through attachment of ubiquitin or ubiquitin-like modifying polypeptide to lysine residues that are important for the protein's activity or structure. For example, a target protein can be a protein involved in cell cycle, signal transduction, cell differentiation, cell dedifferentiation, cell growth, production of cytokines or other biological modifiers, production of regulatory or functional proteins, pro-inflammatory signaling, or the glucose regulation pathway. In one embodiment, a target protein can be a protein that is not known to be ubiquitinated or not known to be a substrate for any ubiquitin pathway protein.

In another embodiment, a target protein can be a disease related protein, e.g., a protein for which changes in its function or activity cause disease, or whose function is considered important to the propagation of the disease state. In some embodiments, the target protein may be either stable or unstable, e.g., G-protein coupled receptor (GPCR), androgen receptor, estrogen receptor, myc, cyclin B, Ras, or cyclin E.

In some embodiments, a target protein can include cyclin A/CDK2, pRB, maltose-binding protein (MBP), β-galactosidase, and GFP-tagged proteins.

### Ubiquitin Pathway Moiety and Binding Peptide Coupling

In some embodiments of the present invention, the mRNA encodes a ubiquitin pathway moiety that is directly fused with the binding protein. The ubiquitin pathway moiety can be an endogenous protein that forms part of the ubiquitin ligase complex, such as an E3 adaptor protein or an E3 ligase. Accordingly, in some embodiments, the mRNA encodes an E3 adaptor or E3 ligase that is fused with a binding protein of interest. In a typical embodiment, the mRNA encodes an E3 ligase in which the endogenous substrate recognition domain has been removed and which is fused to a binding protein (e.g., an antibody that specifically binds the target protein of interest). Suitable E3 ligases include, but are not limited to, SPOP, CHIP, CRBN, VHL, XIAP, MDM2 and cIAP. Using an endogenous protein that forms part of the ubiquitin ligase complex as the ubiquitin pathway moiety is particularly attractive because it can recruit the other components of the ubiquitin ligase complex to the target protein of interest to effect is selective degradation. Moreover, the use of an endogenous protein has the additional advantage that it may avoid the induction of an undesired immune response.

Alternatively, the ubiquitin pathway moiety can be an exogenous protein that binds to an endogenous protein that forms part of the ubiquitin ligase complex. For example, the ubiquitin pathway moiety can be an antibody that specifically binds to an E3 adaptor protein or an E3 ligase. In particular embodiments, the antibody specifically binds an E3 ligase, e.g., an E3 ligase selected from the group consisting of SPOP, CHIP, CRBN, VHL, XIAP, MDM2 and cIAP. Accordingly, in some embodiments, the mRNA encodes an antibody directed to an E3 adaptor or E3 ligase that is fused with a binding protein of interest (e.g., an antibody that specifically binds the target protein of interest). In a specific embodiment, the mRNA encodes an antibody directed to E3 ligase that is fused with a binding protein of interest (e.g., an antibody that specifically binds the target protein of interest). Using an antibody that specifically binds to an E3 adaptor protein or an E3 ligase may be advantageous because of the diversity of ubiquitin ligases and adaptor proteins expressed in the human body. An existing construct could be modified to target a different ligase complex simply by replacing the antibody sequence encoded by the mRNA, e.g., to achieve selective degradation of a target protein of interest in only certain cells that express the ubiquitin ligase targeted by the antibody.

In some embodiments, the mRNA encodes a ubiquitin pathway moiety that fused with the binding protein in the absence of a linker.

In some embodiments, the mRNA encodes a ubiquitin pathway moiety that is coupled, e.g., covalently by any suitable means to the binding peptide. For example, a ubiquitin pathway moiety, for example, an E3 ligase such as SPOP E3 ligase, or an antibody directed to an E3 ligase, is be coupled to a binding peptide of interest. In some embodiments, the composition of the present invention can be a chimeric fusion protein which is encoded by an mRNA expression system. In another embodiment, the ubiquitin pathway moiety is covalently coupled to the binding peptide through a linker, e.g., a linker which has a binding domain for the ubiquitin pathway moiety as well as binding peptide. Any suitable linker known in the art can be used. (See, e.g., Chen et al., Adv Drug Deliv Rev. 2013 Oct. 15; 65(10): 1357-1369, the contents of which are incorporated herein by reference).

In some embodiments, a linker is a flexible linker. In some embodiments, a linker is a rigid linker. In some embodiments, a linker is a helical linker. In some embodiments, a suitable rigid linker is Proline-rich. In some embodiments, a suitable rigid linker comprises PAPAP. In some embodiments, a rigid linker is PAPAP. In some embodiments a suitable helical linker is a rigid helical linker.

In some embodiments, the linker is a GS linker. Various GS linkers are known and the art. For example, in some embodiments, the linker contains (GGS)n, wherein n is 1 to 10, such as 1 to 5, for example 1 to 3, such as GGS(GGS)n, wherein n is 0 to 10. In some embodiments, the linker contains the sequence (GGGGS)n, wherein n is 1 to 10 or n is 1 to 5, such as 1 to 3. In further embodiments, the linker contains (GGGGGS)n, wherein n is 1 to 4, such as 1 to 3. The linker can include combinations of any of the above, such as repeats of 2, 3, 4, or 5 GS, GGS, GGGGS, and/or GGGGGS linkers may be combined. In some embodiments, a linker is 2-30 amino acids in length. In some embodiments, a linker is 2, 3, 4, 5, 6, 7, 8, 9, 10, 11, 12, 13, 14, 15, 16, 17, 18, 19, or 20 amino acids in length.

The linkers can be naturally-occurring, synthetic or a combination of both. Particularly suitable linker polypeptides predominantly include amino acid residues selected from Glycine (Gly), Serine (Ser), Alanine (Ala), and Threonine (Thr). For example, the linker may contain at least 75% (calculated on the basis of the total number of residues present in the peptide linker), such as at least 80%, at least 85%, or at least 90% of amino acid residues selected from Gly, Ser, Ala, and Thr. The linker may also consist of Gly, Ser, Ala and/or Thr residues only. In some embodiments, the linker contains 1-25 glycine residues, 5-20 glycine residues, 5-15 glycine residues, or 8-12 glycine residues. In some aspects, suitable peptide linkers typically contain at least 50% glycine residues, such as at least 75% glycine residues. In some embodiments, a peptide linker comprises glycine residues only. In some embodiments, a peptide linker comprises glycine and serine residues only.

In yet another embodiment, the ubiquitin pathway moiety can be coupled noncovalently to the binding peptide upon the presence of a signal factor, e.g., the presence or the level of an intracellular metabolite, regulatory protein, etc. For example, the ubiquitin pathway moiety and the binding peptide can be coupled when they simultaneously chelate an intracellular metabolite.

In still another embodiment, the ubiquitin pathway moiety can include a first coupling moiety and the binding peptide can include a second coupling moiety such that the first and the second coupling moiety are coupled or bind to each other in the presence of a signal factor or enzymatic activity in vitro or in vivo (e.g., phosphorylation of the first coupling moiety by a kinase that is produced by cancer cells enables it to bind to the second coupling moiety).

Alternatively, in some embodiments, the ubiquitin pathway moiety and the binding peptide may not be separated by a linker, instead they can be part of a single moiety.

Combinations of different ubiquitin pathway moieties and binding peptides can be used to perform target ubiquitination. Such target ubiquitination is useful for regulating protein levels or activities, thus providing therapeutic treatment for disease conditions. This creates an alternative method for the selective degradation of proteins of interest.

One or more mRNAs of the present invention can be administered to ubiquitinate a target protein either in vitro or in vivo. Such ubiquitination by the mRNA encoded protein results in the selective degradation of a protein of interest.

In one embodiment, two or more mRNAs of the present invention encode the same binding peptide, but are coupled with two or more different ubiquitin pathway moieties that are administered to cells to ubiquitinate a target protein, e.g., ubiquitinate a target protein with a desired rate or degree. For example, in some embodiments, a composition comprises two mRNAs, the two of which encode a binding peptide that targets that the same protein of interest, but each of which are coupled with a different ubiquitin pathway moiety (e.g., one mRNA encodes a CHIP E3 ligase, while the other mRNA encodes a SPOP E3 ligase).

In another embodiment two or more mRNAs of the present invention encode the same ubiquitin pathway moiety, but encode different binding peptides that bind to different target proteins. In still another embodiment, the mRNA encoding the ubiquitin targeting moiety and the binding protein is engineered for expression in specific locations within or outside of the cell. This is accomplished, for example, by engineering the mRNA to encode for a signal peptide, such as a nuclear localization signal, an endoplasmic reticulum signal (ER signal), and endoplasmic reticulum retention signal (ER retention signal), or a cell secretory signal. In this manner, a protein of interest can be targeted for degradation in various compartments of a cell, as well as in locations exterior to the cell.

### Cell Delivering Moiety

In some embodiments, the mRNA of the present invention may optionally encode a cell delivering moiety. A cell delivering moiety is any structure that facilitates the delivery of the composition or promotes transduction of the composition into cells. In some embodiments, for example, and as described in greater detail below, the mRNA of the present invention is encapsulated within a lipid nanoparticle. In one embodiment, a cell delivering moiety is derived from virus protein or peptide, e.g., a tat peptide. In another embodiment, a cell delivering moiety is a hydrophobic compound capable of penetrating cell membranes. Alternatively, a ubiquitin pathway protein binding moiety that is more susceptible for cell membrane penetration is used to enhance the cell membrane transduction of the composition.

### Signal Peptides

In some embodiments, the mRNA of the present invention may optionally encode a signal peptide, which can enable a binding peptide to target a protein of interest present at different locations—inside or outside of a cell. In some embodiments, the signal peptide can be one or more of a nuclear localization sequence, an endoplasmic reticulum (ER) signal sequence, an endoplasmic reticulum (ER) retention sequence, or a cell secretory sequence. In some embodiments, an E3 ligase protein naturally contains an NLS sequence. In some embodiments, a NLS is fused to an E3 ligase protein at the N-terminus. In some embodiments, a NLS is fused to an E3 ligase protein at the C-terminus.

A nuclear localization signal or sequence (NLS) is an amino acid sequence that ‘tags’ a protein for import into the cell nucleus by nuclear transport. Typically, this signal typically comprises of one or more short sequences of positively charged lysines or arginines exposed on the protein surface. For example, in some embodiments, an mRNA encoding protein construct described herein contains a NLS that can facilitate ubiquitination of a nuclear protein, and thereby target a protein inside the cell nucleus for degradation. A nuclear localization signal used in the present invention is not particularly limited as far as it has the ability to translocate a substance to which the signal sequence is attached into the nucleus. Various kinds of NLS known in the art are suitable for use with the invention described herein. In some embodiments, a nuclear localization signal can be of SV40 VP1, SV40 large T antigen, or hepatitis D virus δ antigen, or a sequence containing “PKKKRKV” that is the minimum unit having the nuclear translocation activity within the nuclear localization signal of SV40 large T antigen.

In some embodiments, the signal peptide can be an ER signal sequence. ER signal sequence can be an amino acid sequence that directs a protein to the ER membrane of a cell. An mRNA construct that contains an ER signal sequence facilitates ubiquitination of a protein within an endoplasmic reticulum, and thereby target a protein inside or associated with the ER.

In some embodiments, the signal peptide can be an endoplasmic reticulum (ER) retention sequence. The ER retention sequence can be an amino acid sequence that ‘tags’ a protein to be retained within an endoplasmic reticulum. An mRNA construct with an ER retention signal sequence facilitates ubiquitination of a protein within an endoplasmic reticulum, and thereby continuous regulation of the level of a target protein inside an ER.

A monomeric ER signal sequence is a polypeptide where at least a portion of the polypeptide is capable of functioning as an endoplasmic reticulum (ER) routing signal and/or as an endoplasmic reticulum retention signal. An ER routing signal functions to direct a polypeptide to the ER, while a retention signal functions to retain the polypeptide in the ER or to prevent secretion of ER-localized polypeptides.

Various epitopes for use as ER signals or ER retention sequences are known in the art and include for example hemagglutinin (HA), FLAG and Myc, among others.

In some embodiments, the signal peptide can be a cell secretory sequence. An mRNA construct with cell secretory sequence facilitates ubiquitination of a protein disposed outside a cell.

Examples of secretory proteins are discussed below and include proteins with important roles in cell-to-cell signaling. Such proteins include transmembrane receptors and cell surface markers, extracellular matrix molecules, cytokines, hormones, growth and differentiation factors, neuropeptides, vasomediators, ion channels, transporters/pumps, and proteases. (Reviewed in Alberts, B. et al. (1994) Molecular Biology of The Cell, Garland Publishing, New York N.Y., pp. 557-560, 582-592.).

An exemplary mRNA may encode a chimeric fusion protein comprising, starting from the N-terminus, an ER signal sequence, a binding protein that targets a protein of interest (e.g., an antibody), a ubiquitin pathway moiety (e.g., an E3 adaptor protein, E3 ligase, or antibody that specifically binds to an E3 adaptor protein or an E3 ligase), and an ER retention sequence. In other embodiments, an exemplary mRNA encodes a chimeric fusion protein comprising, starting from the N-terminus, a binding protein that targets a protein of interest (e.g., an antibody), a ubiquitin pathway moiety (e.g., an E3 adaptor protein, E3 ligase, or an antibody that specifically binds to an E3 adaptor protein or an E3 ligase), and a NLS.

Administration of the mRNA Compositions

In some embodiments, the mRNA compositions described herein are used for the treatment of a disease. Any kind of disease which is characterized by the aberrant expression, e.g., overexpression, of a protein or peptide can be treated by the mRNA compositions described herein. Diseases, including symptoms thereof, that are associated or caused by aberrant expression or overexpression of proteins or peptides are known in the art and include for example, prion-based diseases, polycystic kidney disease, Pelizaeus-Merzbacher disease, inflammatory diseases, and cancer. In some embodiments, a disease may be associated with one or more mutation in a protein or misfolding/aggregation of protein. For example, the mRNA compositions described herein may be used in a method of treating a disease or disorder associated with or caused by aberrant expression of a target protein. The target protein can be an enzyme, a protein involved in cell signaling, cell division, or metabolism, or a protein involved in an inflammatory response. Accordingly, in some embodiments, thee mRNA compositions described herein may be used in a method of treating cancer, a metabolic disease or an inflammatory disease. In certain embodiments, the invention relates to the use of an mRNA composition described herein in the manufacture of a medicament for treating a disease or disorder associated with or caused by the aberrant expression of a target protein. The composition and method according to this invention can be useful in degrading a protein of interest in combination with other therapies.

The mRNA compositions described herein can result in rapid targeting and degradation of a target protein of interest. In some embodiments, the mRNA compositions described herein result in targeted degradation of a protein of interest within about 48 hours, 40 hours, 36 hours, 32 hours, 28 hours, 24 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, or less than 4 hours following administration to a subject in need thereof. Accordingly, in some embodiments, the mRNA composition results in targeted degradation of a protein of interest within about 24 hours following administration to a subject in need thereof. In some embodiments, the mRNA composition results in targeted degradation of a protein of interest within about 20 hours following administration to a subject in need thereof. The mRNA composition results in targeted degradation of a protein of interest within about 19 hours following administration to a subject in need thereof. The mRNA composition results in targeted degradation of a protein of interest within about 18 hours following administration to a subject in need thereof. The mRNA composition results in targeted degradation of a protein of interest within about 17 hours following administration to a subject in need thereof. The mRNA composition results in targeted degradation of a protein of interest within about 16 hours following administration to a subject in need thereof. The mRNA composition results in targeted degradation of a protein of interest within about 16 hours following administration to a subject in need thereof. The mRNA composition results in targeted degradation of a protein of interest within about 15 hours following administration to a subject in need thereof. The mRNA composition results in targeted degradation of a protein of interest within about 14 hours following administration to a subject in need thereof. The mRNA composition results in targeted degradation of a protein of interest within about 13 hours following administration to a subject in need thereof. The mRNA composition results in targeted degradation of a protein of interest within about 12 hours following administration to a subject in need thereof. The mRNA composition results in targeted degradation of a protein of interest within about 11 hours following administration to a subject in need thereof. The mRNA composition results in targeted degradation of a protein of interest within about 10 hours following administration to a subject in need thereof. The mRNA composition results in targeted degradation of a protein of interest within about 9 hours following administration to a subject in need thereof. The mRNA composition results in targeted degradation of a protein of interest within about 8 hours following administration to a subject in need thereof. The mRNA composition results in targeted degradation of a protein of interest within about 7 hours following administration to a subject in need thereof. The mRNA composition results in targeted degradation of a protein of interest within about 6 hours following administration to a subject in need thereof. The mRNA composition results in targeted degradation of a protein of interest within about 5 hours following administration to a subject in need thereof. The mRNA composition results in targeted degradation of a protein of interest within about 4 hours following administration to a subject in need thereof. The mRNA composition results in targeted degradation of a protein of interest less than 4 hours following administration to a subject in need thereof.

The mRNA compositions of the present invention useful for therapeutic treatment can be administered alone, in a composition with a suitable pharmaceutical carrier, or in combination with other therapeutic agents. An effective amount of the compositions to be administered can be determined on a case-by-case basis.

The compositions of the present invention may be administered in any way which is medically acceptable which may depend on the disease condition or injury being treated. Possible administration routes include injections, by parenteral routes such as intravascular, intravenous, intraepidural or others, as well as oral, nasal, ophthalmic, rectal, topical, or pulmonary, e.g., by inhalation, or by nebulization.

In some embodiments, the administration of the composition results in a reduced level of aberrantly expressed proteins as compared to the control throughout the treatment period. In some embodiments, the administration of the composition results in a reduced level of aberrantly expressed proteins as compared to the control for 5 days. In some embodiments, the administration of the composition results in a reduced level of aberrantly expressed proteins as compared to the control for 7 days. In some embodiments, the administration of the composition results in a reduced level of aberrantly expressed proteins as compared to the control for 10 days. In some embodiments, the administration of the composition results in a reduced level of aberrantly expressed proteins as compared to the control for 15 days. In some embodiments, the administration of the composition results in a reduced level of aberrantly expressed proteins as compared to the control for 20 days. In some embodiments, the administration of the composition results in a reduced level of aberrantly expressed proteins as compared to the control for 25 days. In some embodiments, the administration of the composition results in a reduced level of aberrantly expressed proteins as compared to the control for 30 days. In some embodiments, the administration of the composition results in a reduced level of aberrantly expressed proteins as compared to the control for 35 days. In some embodiments, the administration of the composition results in a reduced level of aberrantly expressed proteins as compared to the control for 40 days. In some embodiments, the administration of the composition results in a reduced level of aberrantly expressed proteins as compared to the control for 45 days.

### Dose and Administration Interval

As used herein, the term “therapeutically effective amount” is largely based on the total amount of the mRNA contained in the compositions of the present invention. Generally, a therapeutically effective amount is sufficient to achieve a meaningful benefit to the subject. For example, a therapeutically effective amount may be an amount sufficient to achieve a desired therapeutic and/or prophylactic effect. Generally, the amount of a therapeutic agent (e.g., mRNA encoding a protein or a peptide) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

A delivery vehicle comprising mRNA may be administered and dosed in accordance with current medical practice, taking into account the clinical condition of the subject, the site and method of administration (e.g., local and systemic, including intratumoral, intravenous, and via injection), the scheduling of administration, the subject's age, sex, body weight, and other factors relevant to clinicians of ordinary skill in the art. The “effective amount” for the purposes herein may be determined by such relevant considerations as are known to those of ordinary skill in experimental clinical research, pharmacological, clinical and medical arts.

In some embodiments, the method comprises injecting a single dose. In some embodiments, the method comprises injecting multiple doses periodically.

Provided methods of the present invention contemplate single as well as multiple administrations of a therapeutically effective amount of the composition described herein. The composition can be administered at regular intervals, depending on the nature, severity and extent of the subject's condition. In some embodiments, a therapeutically effective amount of the composition of the present invention may be administered periodically at regular intervals (e.g., daily, twice a week, once every four days, weekly, once every 10 days, biweekly, monthly, bimonthly, twice a month, once every 30 days, once every 28 days or continuously.

In some embodiments, provided liposomes and/or compositions are formulated such that they are suitable for extended-release of the mRNA contained therein. Such extended-release compositions may be conveniently administered to a subject at extended dosing intervals. For example, in some embodiments, the compositions of the present invention are administered to a subject twice a day. In some embodiments, the composition is administered to a subject twice a day. In some embodiments, the composition is administered to a subject daily. In some embodiments, the composition is administered to a subject every other day. In some embodiments, the composition is administered to a subject twice a week. In some embodiments, the composition is administered to a subject once a week. In some embodiments, the composition is administered to a subject once every 7 days. In some embodiments, the composition is administered to a subject once every 10 days. In some embodiments, the composition is administered to a subject once every 14 days. In some embodiments, the composition is administered to a subject once every 28 days. In some embodiments, the composition is administered to a subject once every 30 days. In some embodiments, the composition is administered to a subject once every two weeks. In some embodiments, the composition is administered to a subject once every three weeks. In some embodiments, the composition is administered to a subject once every four weeks. In some embodiments, the composition is administered to a subject once a month. In some embodiments, the composition is administered to a subject twice a month. In some embodiments, the composition is administered to a subject once every six weeks. In some embodiments, the composition is administered to a subject once every eight weeks. In some embodiments, the composition is administered to a subject once every other month. In some embodiments, the composition is administered to a subject once every three months. In some embodiments, the composition is administered to a subject once every four months. In some embodiments, the composition is administered to a subject once every six months. In some embodiments, the composition is administered to a subject once every eight months. In some embodiments, the composition is administered to a subject once every nine months. In some embodiments, the composition is administered to a subject annually. Also contemplated are compositions and liposomes which are formulated for depot administration (e.g., intramuscularly, subcutaneously, intravitreally) to either deliver or release mRNA over extended periods of time. Preferably, the extended-release means employed are combined with modifications made to the mRNA to enhance stability.

A therapeutically effective amount is commonly administered in a dosing regimen that may comprise multiple unit doses. For any particular vaccine, a therapeutically effective amount and administration interval (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific composition employed; the specific composition employed; the age, body weight, general health, sex and diet of the patient; the time of administration, route of administration, and/or rate of excretion or metabolism of the specific protein employed; the duration of the treatment; and like factors as is well known in the medical arts.

In some embodiments, an initial dose and the subsequent dose or doses are same in amount. In some embodiments, an initial dose and the subsequent dose or doses are different in amount. In some embodiments, an initial dose is greater than the subsequent dose or doses. In some embodiments, an initial dose is less than the subsequent dose or doses. In some embodiments each of the multiple doses comprise the same dosage amount of mRNA. In some embodiments, each of the multiple doses comprise a different dosage amount of mRNA.

### Composition of Invention

In one aspect, the present invention relates to methods for selective degradation of aberrantly expressed or overly expressed proteins via administration of a composition comprising one or more mRNAs encoding a protein or a peptide encapsulated within lipid nanoparticles. In one aspect, the present invention provides a pharmaceutical composition, comprising one or more mRNAs each encoding a ubiquitin pathway moiety, a binding peptide, and optionally a signal peptide, wherein one or more mRNAs are encapsulated within lipid nanoparticles.

Synthesis of mRNA

In some embodiments, the one or more mRNAs encode a ubiquitin pathway moiety, a binding peptide, and optionally a signal peptide.

In some embodiments, the one or more mRNAs are codon optimized. In some embodiments, the protein or the peptide encoded by the mRNAs are wild-type. In some embodiments, the protein or the peptide encoded by the mRNAs contain a mutation or modification.

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

TABLE A
Exemplary Nucleotide Sequences

| | |
|---|---|
| ΔSPOP mRNA | AGCGTGAACATTAGCGGGCAGAATACCATGAACATGGTCA |
| coding sequence | AAGTGCCGGAATGTCGCCTGGCCGACGAACTGGGCGGCCT |
| | GTGGGAAAACTCAAGGTTCACGGACTGCTGCCTTTGCGTG |
| | GCCGGCCAAGAATTCCAGGCCCATAAGGCCATCCTGGCCG |
| | CGCGGTCGCCAGTATTCTCGGCCATGTTCGAACACGAAAT |
| | GGAAGAGTCTAAGAAGAATAGAGTGGAAATCAACGATGT |
| | GGAGCCTGAGGTCTTTAAGGAAATGATGTGCTTTATATACA |
| | CTGGAAAGGCCCCCAACCTCGACAAGATGGCCGACGACTT |
| | GCTGGCTGCCGCCGACAAATACGCCCTGGAGCGGCTCAAG |
| | GTTATGTGCGAGGACGCGCTGTGCAGCAACCTCAGCGTGG |
| | AGAACGCCGCAGAAATCCTCATCCTGGCGGATTTGCACTC |
| | CGCCGACCAACTCAAGACCCAGGCCGTGGACTTCATTAAC |
| | TACCACGCTTCCGACGTGCTGGAGACTTCCGGATGGAAGT |
| | CCATGGTCGTCAGCCACCCGCACTTAGTGGCAGAGGCCTA |
| | CAGATCCCTGGCCAGTGCCCAGTGCCCTTTCCTGGGGCCGC |
| | CTAGGAAACGCCTGAAGCAGAGCGGGGGTGGCTCC (SEQ |
| | ID NO: 1) |
| hVHL mRNA | GGTGGTGGATCCGGCGGCGGCTCCATGCCTAGGAGAGC |
| coding sequence | GGAGAATTGGGACGAAGCAGAAGTCGGAGCAGAAGAAGC |
| | CGGAGTGGAAGAATACGGACCTGAAGAGGACGGGGGAGA |
| | AGAGTCGGGCGCCGAAGAGTCCGGCCCCGAGGAGTCCGGA |
| | CCCGAAGAACTGGGCGCCGAGGAAGAAATGGAGGCCGGG |
| | CGCCCTAGGCCGGTGCTGCGGTCCGTGAACTCCCGCGAGC |
| | CGAGCCAGGTCATTTTCTGCAATCGCAGCCCGAGAGTGGT |
| | GCTGCCCGTGTGGCTGAACTTTGACGGGGAGCCTCAGCCA |
| | TACCCTACCCTGCCACCGGGAACTGGACGCAGAATCCACA |
| | GCTACCGGGGCCACCTTTGGCTTTTCCGGGACGCCGGGACT |
| | CACGACGGGCTGCTCGTGAACCAGACCGAGTTGTTCGTGC |
| | CGTCCCTGAACGTCGATGGCCAGCCAATTTTCGCCAACATC |
| | ACCCTGCCGGTGTACACACTGAAGGAACGGTGCCTCCAAG |
| | TCGTCAGAAGCCTCGTCAAGCCCGAGAACTACCGGCGGCT |
| | GGACATCGTGCGGTCACTCTACGAAGATCTCGAGGACCAC |
| | CCTAACGTGCAAAAGGACCTGGAGAGGCTGACTCAGGAAC |
| | GCATCGCCCATCAACGCATGGGCGACGGTGGTGGCTCC |
| | (SEQ ID NO: 2) |
| ΔCHIP mRNA | GGTTCCGGCTCTGGACGGCTGAACTTCGGGGACGATATT |
| coding sequence | CCTAGCGCCCTGCGCATCGCCAAGAAGAAGAGATGGAACT |
| | CAATCGAGGAACGGCGAATCCACCAGGAGTCCGAGCTGCA |
| | TAGCTACCTTAGCCGCCTTATCGCCGCGGAACGGGAGAGG |
| | GAGCTGGAAGAGTGTCAGCGGAACCATGAGGGCGACGAA |
| | GATGACTCCCACGTCCGGGCACAGCAGGCCTGCATCGAGG |
| | CTAAGCACGACAAGTACATGGCCGATATGGACGAGTTATT |
| | CAGCCAAGTGGACGAGAAGCGGAAGAAGCGCGACATCCC |
| | GGACTACTTATGCGGAAAGATTTCCTTCGAACTTATGAGGG |
| | AACCGTGTATCACCCCGTCCGGGATCACCTACGACCGGAA |
| | AGACATCGAAGAACACCTACAGCGCGTGGGGCACTTCGAC |
| | CCGGTCACCCGGAGCCCGCTGACCCAAGAGCAATTAATCC |
| | CCAACTTGGCGATGAAGGAAGTGATCGACGCCTTCATTAG |
| | CGAAAATGGATGGGTGGAGGATTACGGGGGTGGCTCC |
| | (SEQ ID NO: 3) |
| ER signal peptide | GGCTGGTCTTGCATTATACTCTTCCTTGTCGCCACCGCCAC |
| mRNA coding | TGGAGCGCATAGC (SEQ ID NO: 4) |
| sequence | |
| ER retention signal | TCCGAGAAAGATGAACTG (SEQ ID NO: 5) |
| mRNA coding | |
| sequence | |

NLS = underlined; Linker = bolded;

TABLE B
Exemplary Amino Acid Sequences

| | |
|---|---|
| ΔSPOP Protein | SVNISGQNTMNMVKVPECRLADELGGLWENSRFTDCCLCVA |
| Sequence | GQEFQAHKAILAARSPVFSAMFEHEMEESKKNRVEINDVEPE |
| | VFKEMMCFIYTGKAPNLDKMADDLLAAADKYALERLKVMC |
| | EDALCSNLSVENAAEILILADLHSADQLKTQAVDFINYHASDV |
| | LETSGWKSMVVSHPHLVAEAYRSLASAQCPFLGPPRKRLKQS |
| | GGGS (SEQ ID NO: 6) |
| hVHL Protein | GGGSGGGSMPRRAENWDEAEVGAEEAGVEEYGPEEDGGEE |
| Sequence | SGAEESGPEESGPEELGAEEEMEAGRPRPVLRSVNSREPSQVIF |
| | CNRSPRVVLPVWLNFDGEPQPYPTLPPGTGRRIHSYRGHLWL |
| | FRDAGTHDGLLVNQTELFVPSLNVDGQPIFANITLPVYTLKER |
| | CLQVVRSLVKPENYRRLDIVRSLYEDLEDHPNVQKDLERLTQ |
| | ERIAHQRMGDGGGS (SEQ ID NO: 7) |
| ΔCHIP Protein | GSGSGRLNFGDDIPSALRIAKKKRWNSIEERRIHQESELHSYL |
| Sequence | SRLIAAERERELEECQRNHEGDEDDSHVRAQQACIEAKHDKY |
| | MADMDELFSQVDEKRKKRDIPDYLCGKISFELMREPCITPSGI |
| | TYDRKDIEEHLQRVGHFDPVTRSPLTQEQLIPNLAMKEVIDAF |
| | ISENGWVEDYGGGS (SEQ ID NO: 8) |
| ER signal peptide | GWSCIILFLVATATGAHS (SEQ ID NO: 9) |
| Sequence | |
| ER retention | SEKDEL (SEQ ID NO: 10) |
| signal | |
| Sequence | |

NLS = underlined; Linker = bolded;

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

Exemplary Construct Design for mRNAs

Construct design:
X-mRNA coding region-Y
5′ and 3′ UTR Sequences:
X (5′ UTR Sequence) =
(SEQ ID NO: 11)
GGACAGAUCGCCUGGAGACGCCAUCCACGCUGUUUUGACCUCCAUAGAAGA
CACCGGGACCGAUCCAGCCUCCGCGGCCGGGAACGGUGCAUUGGAACGCGG
AUUCCCCGUGCCAAGAGUGACUCACCGUCCUUGACACG
Y (3′ UTR Sequence) =
(SEQ ID NO: 12)
CGGGUGGCAUCCCUGUGACCCCUCCCCAGUGCCUCUCCUGGCCCUGGAAGU
UGCCACUCCAGUGCCCACCAGCCUUGUCCUAAUAAAAUUAAGUUGCAUCAA
GCU
OR
(SEQ ID NO: 13)
GGGUGGCAUCCCUGUGACCCCUCCCCAGUGCCUCUCCUGGCCCUGGAAGUU
GCCACUCCAGUGCCCACCAGCCUUGUCCUAAUAAAAUUAAGUUGCAUCAAA
GCU

The present invention may be used to deliver mRNAs of a variety of lengths. In some embodiments, the present invention may be used to deliver in vitro synthesized mRNA of or greater than about 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, or 20 kb in length. In some embodiments, the present invention may be used to deliver in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

Synthesis of mRNA Using SP6 RNA Polymerase

In some embodiments, mRNA is produced using SP6 RNA Polymerase. SP6 RNA Polymerase is a DNA-dependent RNA polymerase with high sequence specificity for SP6 promoter sequences. The SP6 polymerase catalyzes the 5′→3′ in vitro synthesis of RNA on either single-stranded DNA or double-stranded DNA downstream from its promoter; it incorporates native ribonucleotides and/or modified ribonucleotides and/or labeled ribonucleotides into the polymerized transcript. Examples of such labeled ribonucleotides include biotin-, fluorescein-, digoxigenin-, aminoallyl-, and isotope-labeled nucleotides.

The sequence for bacteriophage SP6 RNA polymerase was initially described (GenBank: Y00105.1) as having the following amino acid sequence:

(SEQ ID NO: 14)
MQDLHAIQLQLEEEMFNGGIRRFEADQQRQIAAGSESDTAWNRRLLSELI
APMAEGIQAYKEEYEGKKGRAPRALAFLQCVENEVAAYITMKVVMDMLNT
DATLQAIAMSVAERIEDQVRFSKLEGHAAKYFEKVKKSLKASRTKSYRHA
HNVAVVAEKSVAEKDADFDRWEAWPKETQLQIGTTLLEILEGSVFYNGEP
VFMRAMRTYGGKTIYYLQTSESVGQWISAFKEHVAQLSPAYAPCVIPPRP
WRTPFNGGFHTEKVASRIRLVKGNREHVRKLTQKQMPKVYKAINALQNTQ
WQINKDVLAVIEEVIRLDLGYGVPSFKPLIDKENKPANPVPVEFQHLRGR
ELKEMLSPEQWQQFINWKGECARLYTAETKRGSKSAAVVRMVGQARKYSA
FESIYFVYAMDSRSRVYVQSSTLSPQSNDLGKALLRFTEGRPVNGVEALK
WFCINGANLWGWDKKTFDVRVSNVLDEEFQDMCRDIAADPLTFTQWAKAD
APYEFLAWCFEYAQYLDLVDEGRADEFRTHLPVHQDGSCSGIQHYSAMLR
DEVGAKAVNLKPSDAPQDIYGAVAQVVIKKNALYMDADDATTFTSGSVTL
SGTELRAMASAWDSIGITRSLTKKPVMTLPYGSTRLTCRESVIDYIVDLE
EKEAQKAVAEGRTANKVHPFEDDRQDYLTPGAAYNYMTALIWPSISEVVK
APIVAMKMIRQLARFAAKRNEGLMYTLPTGFILEQKIMATEMLRVRTCLM
GDIKMSLQVETDIVDEAAMMGAAAPNFVHGHDASHLILTVCELVDKGVTS
IAVIHDSFGTHADNTLTLRVALKGQMVAMYIDGNALQKLLEEHEVRWMVD
TGIEVPEQGEFDLNEIMDSEYVFA.

An SP6 RNA polymerase suitable for the present invention can be any enzyme having substantially the same polymerase activity as bacteriophage SP6 RNA polymerase. Thus, in some embodiments, an SP6 RNA polymerase suitable for the present invention may be modified from SEQ ID NO: 14. For example, a suitable SP6 RNA polymerase may contain one or more amino acid substitutions, deletions, or additions. In some embodiments, a suitable SP6 RNA polymerase has an amino acid sequence about 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, or 60% identical or homologous to SEQ ID NO: 14. In some embodiments, a suitable SP6 RNA polymerase may be a truncated protein (from N-terminus, C-terminus, or internally) but retain the polymerase activity. In some embodiments, a suitable SP6 RNA polymerase is a fusion protein.

An SP6 RNA polymerase suitable for the invention may be a commercially-available product, e.g., from Aldevron, Ambion, New England Biolabs (NEB), Promega, and Roche. The SP6 may be ordered and/or custom designed from a commercial source or a non-commercial source according to the amino acid sequence of SEQ ID NO: 14 or a variant of SEQ ID NO: 14 as described herein. The SP6 may be a standard-fidelity polymerase or may be a high-fidelity/high-efficiency/high-capacity which has been modified to promote RNA polymerase activities, e.g., mutations in the SP6 RNA polymerase gene or post-translational modifications of the SP6 RNA polymerase itself. Examples of such modified SP6 include SP6 RNA Polymerase-Plus™ from Ambion, HiScribe SP6 from NEB, and RiboMAX™ and Riboprobe® Systems from Promega.

In some embodiments, a suitable SP6 RNA polymerase is a fusion protein. For example, an SP6 RNA polymerase may include one or more tags to promote isolation, purification, or solubility of the enzyme. A suitable tag may be located at the N-terminus, C-terminus, and/or internally. Non-limiting examples of a suitable tag include Calmodulin-binding protein (CBP); Fasciola hepatica 8-kDa antigen (Fh8); FLAG tag peptide; glutathione-S-transferase (GST); Histidine tag (e.g., hexahistidine tag (His6)); maltose-binding protein (MBP); N-utilization substance (NusA); small ubiquitin related modifier (SUMO) fusion tag; Streptavidin binding peptide (STREP); Tandem affinity purification (TAP); and thioredoxin (TrxA). Other tags may be used in the present invention. These and other fusion tags have been described, e.g., Costa et al. Frontiers in Microbiology 5 (2014): 63 and in PCT/US16/57044, the contents of which are incorporated herein by reference in their entireties. In certain embodiments, a His tag is located at SP6's N-terminus.

SP6 Promoter

Any promoter that can be recognized by an SP6 RNA polymerase may be used in the present invention. Typically, an SP6 promoter comprises 5′ ATTTAGGTGACACTATAG-3′ (SEQ ID NO: 15). Variants of the SP6 promoter have been discovered and/or created to optimize recognition and/or binding of SP6 to its promoter. Non-limiting variants include but are not limited to:

(SEQ ID NO: 16 to SEQ ID NO: 25)
5′-ATTTAGGGGACACTATAGAAGAG-3′;
5′-ATTTAGGGGACACTATAGAAGG-3′;
5′-ATTTAGGGGACACTATAGAAGGG-3′;
5′-ATTTAGGTGACACTATAGAA-3′;
5′-ATTTAGGTGACACTATAGAAGA-3′;
5′-ATTTAGGTGACACTATAGAAGAG-3′;
5′-ATTTAGGTGACACTATAGAAGG-3′;
5′-ATTTAGGTGACACTATAGAAGGG-3′;
5′-ATTTAGGTGACACTATAGAAGNG-3′;
and
5′-CATACGATTTAGGTGACACTATAG-3′.

In addition, a suitable SP6 promoter for the present invention may be about 95%, 90%, 85%, 80%, 75%, or 70% identical or homologous to any one of SEQ ID NO: 15 to SEQ ID NO: 25. Moreover, an SP6 promoter useful in the present invention may include one or more additional nucleotides 5′ and/or 3′ to any of the promoter sequences described herein.

DNA Template

Typically, a DNA template is either entirely double-stranded or mostly single-stranded with a double-stranded SP6 promoter sequence.

Linearized plasmid DNA (linearized via one or more restriction enzymes), linearized genomic DNA fragments (via restriction enzyme and/or physical means), PCR products, and/or synthetic DNA oligonucleotides can be used as templates for in vitro transcription with SP6, provided that they contain a double-stranded SP6 promoter upstream (and in the correct orientation) of the DNA sequence to be transcribed.

In some embodiments, the linearized DNA template has a blunt-end.

In some embodiments, the DNA sequence to be transcribed may be optimized to facilitate more efficient transcription and/or translation. For example, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., IRES), premature polyA sites, Shine-Dalgarno (SD) sequences, and/or other elements relevant to translation; and/or the DNA sequence may be optimized regarding codon context, codon-anticodon interaction, translational pause sites, and/or other elements relevant to protein folding. Optimization methods known in the art may be used in the present invention, e.g., GeneOptimizer by ThermoFisher and OptimumGene™, which are described in US 20110081708, the contents of which are incorporated herein by reference in its entirety.

In some embodiments, the DNA template includes a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

Large-Scale mRNA Synthesis

The present invention relates to large-scale production of wild-type or codon optimized mRNAs. In some embodiments, a method according to the invention synthesizes mRNA at least 100 mg, 150 mg, 200 mg, 300 mg, 400 mg, 500 mg, 600 mg, 700 mg, 800 mg, 900 mg, 1 g, 5 g, 10 g, 25 g, 50 g, 75 g, 100 g, 250 g, 500 g, 750 g, 1 kg, 5 kg, 10 kg, 50 kg, 100 kg, 1000 kg, or more at a single batch. As used herein, the term “batch” refers to a quantity or amount of mRNA synthesized at one time, e.g., produced according to a single manufacturing setting. A batch may refer to an amount of mRNA synthesized in one reaction that occurs via a single aliquot of enzyme and/or a single aliquot of DNA template for continuous synthesis under one set of conditions. mRNA synthesized at a single batch would not include mRNA synthesized at different times that are combined to achieve the desired amount. Generally, a reaction mixture includes SP6 RNA polymerase, a linear DNA template, and an RNA polymerase reaction buffer (which may include ribonucleotides or may require addition of ribonucleotides).

According to the present invention, 1-100 mg of SP6 polymerase is typically used per gram (g) of mRNA produced. In some embodiments, about 1-90 mg, 1-80 mg, 1-60 mg, 1-50 mg, 1-40 mg, 10-100 mg, 10-80 mg, 10-60 mg, 10-50 mg of SP6 polymerase is used per gram of mRNA produced. In some embodiments, about 5-20 mg of SP6 polymerase is used to produce about 1 gram of mRNA. In some embodiments, about 0.5 to 2 grams of SP6 polymerase is used to produce about 100 grams of mRNA. In some embodiments, about 5 to 20 grams of SP6 polymerase is used to about 1 kilogram of mRNA. In some embodiments, at least 5 mg of SP6 polymerase is used to produce at least 1 gram of mRNA. In some embodiments, at least 500 mg of SP6 polymerase is used to produce at least 100 grams of mRNA. In some embodiments, at least 5 grams of SP6 polymerase is used to produce at least 1 kilogram of mRNA. In some embodiments, about 10 mg, 20 mg, 30 mg, 40 mg, 50 mg, 60 mg, 70 mg, 80 mg, 90 mg, or 100 mg of plasmid DNA is used per gram of mRNA produced. In some embodiments, about 10-30 mg of plasmid DNA is used to produce about 1 gram of mRNA. In some embodiments, about 1 to 3 grams of plasmid DNA is used to produce about 100 grams of mRNA. In some embodiments, about 10 to 30 grams of plasmid DNA is used to about 1 kilogram of mRNA. In some embodiments, at least 10 mg of plasmid DNA is used to produce at least 1 gram of mRNA. In some embodiments, at least 1 gram of plasmid DNA is used to produce at least 100 grams of mRNA. In some embodiments, at least 10 grams of plasmid DNA is used to produce at least 1 kilogram of mRNA.

In some embodiments, the concentration of the SP6 RNA polymerase in the reaction mixture may be from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the SP6 RNA polymerase is from about 10 to 50 nM, 20 to 50 nM, or 30 to 50 nM. A concentration of 100 to 10000 Units/ml of the SP6 RNA polymerase may be used, as examples, concentrations of 100 to 9000 Units/ml, 100 to 8000 Units/ml, 100 to 7000 Units/ml, 100 to 6000 Units/ml, 100 to 5000 Units/ml, 100 to 1000 Units/ml, 200 to 2000 Units/ml, 500 to 1000 Units/ml, 500 to 2000 Units/ml, 500 to 3000 Units/ml, 500 to 4000 Units/ml, 500 to 5000 Units/ml, 500 to 6000 Units/ml, 1000 to 7500 Units/ml, and 2500 to 5000 Units/ml may be used.

The concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about 10 mM, between about 1 mM and about 8 mM, between about 1 mM and about 6 mM, between about 3 mM and about 10 mM, between about 3 mM and about 8 mM, between about 3 mM and about 6 mM, between about 4 mM and about 5 mM. In some embodiments, each ribonucleotide is at about 5 mM in a reaction mixture. In some embodiments, the total concentration of rNTPs (for example, ATP, GTP, CTP and UTPs combined) used in the reaction range between 1 mM and 40 mM. In some embodiments, the total concentration of rNTPs (for example, ATP, GTP, CTP and UTPs combined) used in the reaction range between 1 mM and 30 mM, or between 1 mM and 28 mM, or between 1 mM to 25 mM, or between 1 mM and 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride.

The pH of the reaction mixture may be between about 6 to 8.5, from 6.5 to 8.0, from 7.0 to 7.5, and in some embodiments, the pH is 7.5.

Linear or linearized DNA template (e.g., as described above and in an amount/concentration sufficient to provide a desired amount of RNA), the RNA polymerase reaction buffer, and SP6 RNA polymerase are combined to form the reaction mixture. The reaction mixture is incubated at between about 37° C. and about 42° C. for thirty minutes to six hours, e.g., about sixty to about ninety minutes.

In some embodiments, about 5 mM NTPs, about 0.05 mg/mL SP6 polymerase, and about 0.1 mg/ml DNA template in a suitable RNA polymerase reaction buffer (final reaction mixture pH of about 7.5) is incubated at about 37° C. to about 42° C. for sixty to ninety minutes.

In some embodiments, a reaction mixture contains linearized double stranded DNA template with an SP6 polymerase-specific promoter, SP6 RNA polymerase, RNase inhibitor, pyrophosphatase, 29 mM NTPs, 10 mM DTT and a reaction buffer (when at 10× is 800 mM HEPES, 20 mM spermidine, 250 mM MgCl₂, pH 7.7) and quantity sufficient (QS) to a desired reaction volume with RNase-free water; this reaction mixture is then incubated at 37° C. for 60 minutes. The polymerase reaction is then quenched by addition of DNase I and a DNase I buffer (when at 10× is 100 mM Tris-HCl, 5 mM MgCl₂ and 25 mM CaCl₂), pH 7.6) to facilitate digestion of the double-stranded DNA template in preparation for purification. This embodiment has been shown to be sufficient to produce 100 grams of mRNA.

In some embodiments, a reaction mixture includes NTPs at a concentration ranging from 1-10 mM, DNA template at a concentration ranging from 0.01-0.5 mg/ml, and SP6 RNA polymerase at a concentration ranging from 0.01-0.1 mg/ml, e.g., the reaction mixture comprises NTPs at a concentration of 5 mM, the DNA template at a concentration of 0.1 mg/ml, and the SP6 RNA polymerase at a concentration of 0.05 mg/ml.

Nucleotides

Various naturally-occurring or modified nucleosides may be used to product mRNA according to the present invention. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“ivU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published US Application No. US 2016/0032356 and U.S. Provisional Application 62/464,327, filed Feb. 27, 2017, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized according to the present invention may be used without further purification. In particular, mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art. In some embodiments, the mRNA is purified using Tangential Flow Filtration. Suitable purification methods include those described in US 2016/0040154, US 2015/0376220, PCT application PCT/US18/19954 entitled “METHODS FOR PURIFICATION OF MESSENGER RNA” filed on Feb. 27, 2018, and PCT application PCT/US18/19978 entitled “METHODS FOR PURIFICATION OF MESSENGER RNA” filed on Feb. 27, 2018, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

Characterization of mRNA

Full-length or abortive transcripts of mRNA may be detected and quantified using any methods available in the art. In some embodiments, the synthesized mRNA molecules are detected using blotting, capillary electrophoresis, chromatography, fluorescence, gel electrophoresis, HPLC, silver stain, spectroscopy, ultraviolet (UV), or UPLC, or a combination thereof. Other detection methods known in the art are included in the present invention. In some embodiments, the synthesized mRNA molecules are detected using UV absorption spectroscopy with separation by capillary electrophoresis. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

In some embodiments, mRNA generated by the method disclosed herein comprises less than 10%, less than 9%, less than 8%, less than 7%, less than 6%, less than 5%, less than 4%, less than 3%, less than 2%, less than 1%, less than 0.5%, less than 0.1% impurities other than full length mRNA. The impurities include IVT contaminants, e.g., proteins, enzymes, free nucleotides and/or shortmers.

In some embodiments, mRNA produced according to the invention is substantially free of shortmers or abortive transcripts. In particular, mRNA produced according to the invention contains undetectable level of shortmers or abortive transcripts by capillary electrophoresis or Glyoxal gel electrophoresis. As used herein, the term “shortmers” or “abortive transcripts” refers to any transcripts that are less than full-length. In some embodiments, “shortmers” or “abortive transcripts” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail.

Delivery Vehicles

According to the present invention, mRNA encoding a protein or a peptide (e.g., a full length, fragment, or portion of a protein or a peptide) as described herein may be delivered as naked RNA (unpackaged) or via delivery vehicles. As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

In some embodiments, a delivery vehicle comprising one or more mRNAs is administered by intravenous, intratumoral, intradermal, subcutaneous, intramuscular, intraperitoneal, epideural, intrathecal, or pulmonary delivery, e.g., comprising nebulization. In some embodiments, the mRNA is expressed in the tissue in which the delivery vehicle was administered. Additional teaching of pulmonary delivery and nebulization are described in the related international application PCT/US17/61100 filed Nov. 10, 2017 by Applicant entitled “NOVEL ICE-BASED LIPID NANOPARTICLE FORMULATION FOR DELIVERY OF MRNA”, and the U.S. Provisional Application Ser. No. 62/507,061, each of which is incorporated by reference in its entirety.

In some embodiments, mRNAs encoding a protein or a peptide may be delivered via a single delivery vehicle. In some embodiments, mRNAs encoding a protein or a peptide may be delivered via one or more delivery vehicles each of a different composition. In some embodiments, the one or more mRNAs are encapsulated within the same lipid nanoparticles. In some embodiments, the one or more mRNAs are encapsulated within separate lipid nanoparticles.

According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags. Also contemplated is the use of bionanocapsules and other viral capsid proteins assemblies as a suitable transfer vehicle. (Hum. Gene Ther. 2008 September; 19(9):887-95).

Liposomal Delivery Vehicles

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired mRNA to a target cell or tissue. In some embodiments, a nanoparticle delivery vehicle is a liposome. In some embodiments, a liposome comprises one or more cationic lipids, one or more non-cationic lipids, one or more cholesterol-based lipids and one or more PEG-modified lipids. In some embodiments, a liposome comprises no more than three distinct lipid components. In some embodiments, one distinct lipid component is a sterol-based cationic lipid.

Cationic Lipids

As used herein, the phrase “cationic lipids” refers to any of a number of lipid species that have a net positive charge at a selected pH, such as physiological pH.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

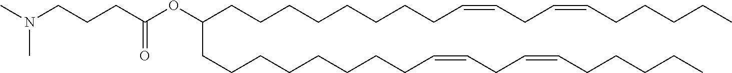 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

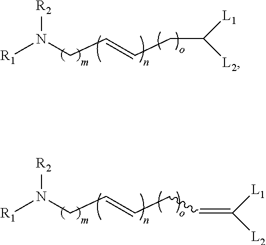

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z, 18Z)—N,N-dimethyl-6-(9Z,12Z)-octadeca-9,12-dien-1-yl)tetracosa-15,18-dien-1-amine (“HGT5000”), having a compound structure of:

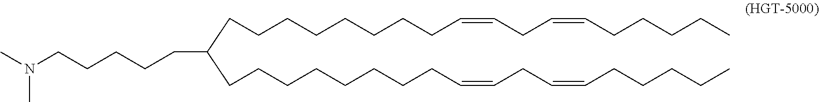 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z, 18Z)—N,N-dimethyl-6-((9Z,12Z)-octadeca-9,12-dien-1-yl) tetracosa-4,15,18-trien-1-amine (“HGT5001”), having a compound structure of:

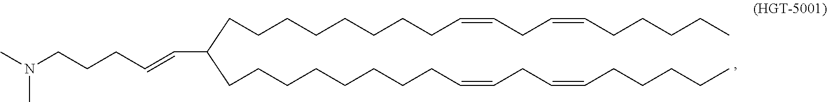 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)—N,N-dimethyl-6-((9Z,12Z)-octadeca-9,12-dien-1-yl) tetracosa-5,15,18-trien-1-amine (“HGT5002”), having a compound structure of:

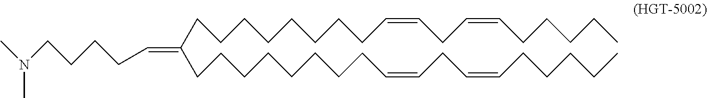 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

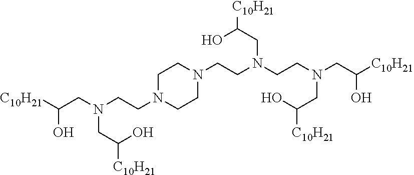

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

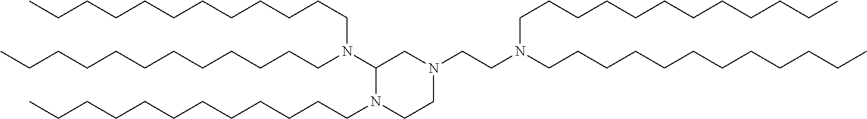 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

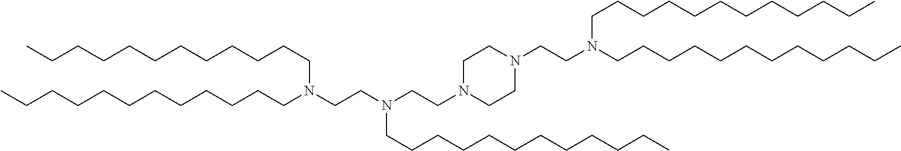 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

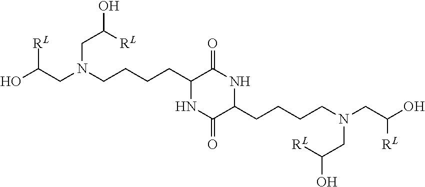

or pharmaceutically acceptable salts thereof, wherein each instance of R^{L} is independently optionally substituted C₆-C₄₀ alkenyl. In certain embodiments, the compositions and methods of the present invention include a cationic lipid having a compound structure of:

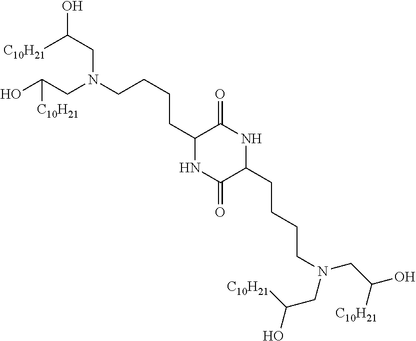

and pharmaceutically acceptable salts thereof. In certain embodiments, the compositions and methods of the present invention include a cationic lipid having a compound structure of:

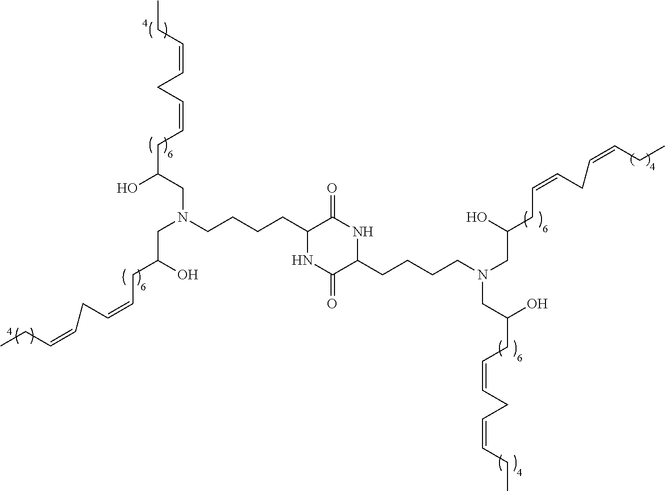

and pharmaceutically acceptable salts thereof. In certain embodiments, the compositions and methods of the present invention include a cationic lipid having a compound structure of:

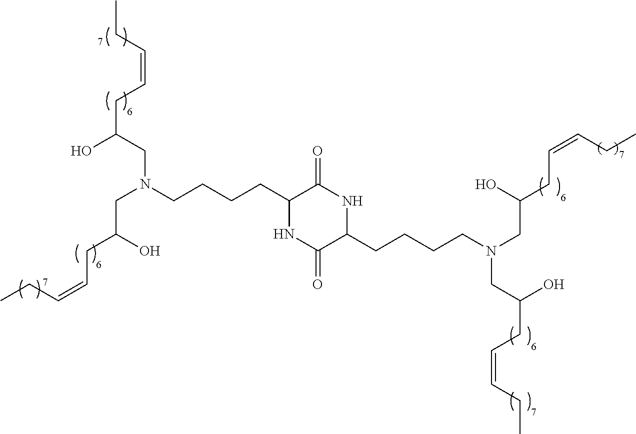

and pharmaceutically acceptable salts thereof. In certain embodiments, the compositions and methods of the present invention include a cationic lipid having a compound structure of:

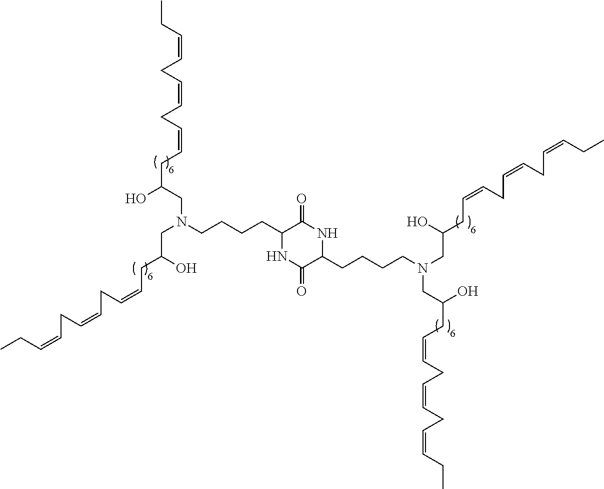

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

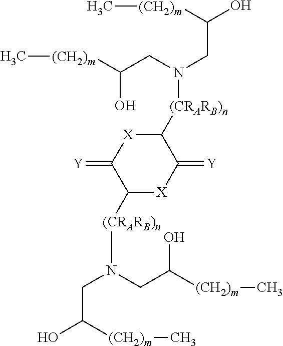

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R_{B} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present invention include a cationic lipid, “Target 23”, having a compound structure of:

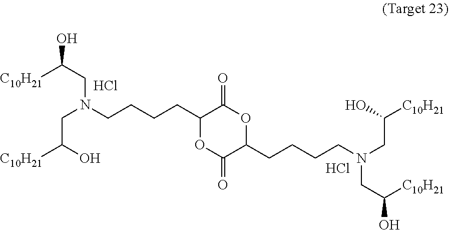

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

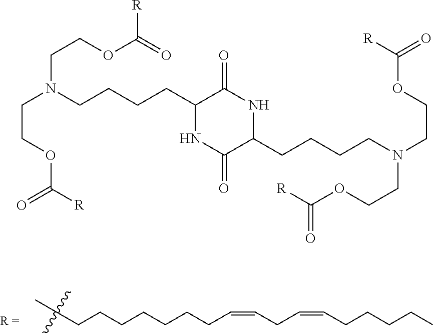

or a pharmaceutically acceptable salt thereof. In some embodiments, the compositions and methods of the present invention include a cationic lipid having the compound structure:

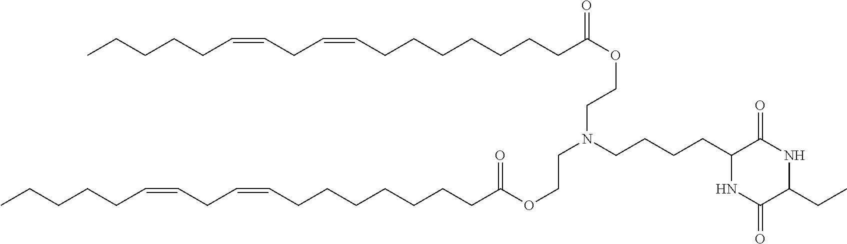 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

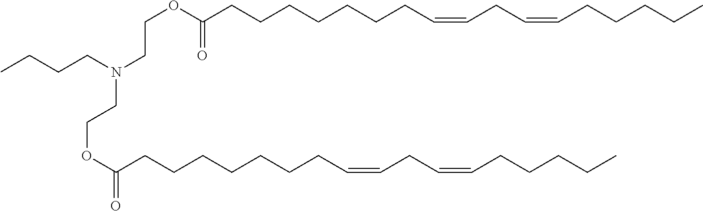 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include cationic lipids as described in U.S. Provisional Patent Application Ser. No. 62/758,179, which is incorporated herein by reference. In some embodiments, the compositions and methods of the present invention include a cationic lipid of the following formula:

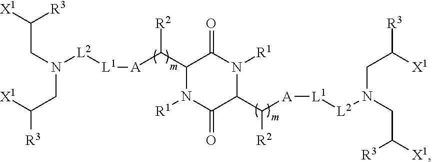

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the compositions and methods of the present invention include a cationic lipid of the following formula:

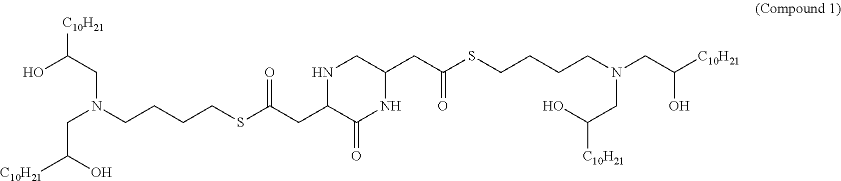

or a pharmaceutically acceptable salt thereof. In some embodiments, the compositions and methods of the present invention include a cationic lipid of the following formula:

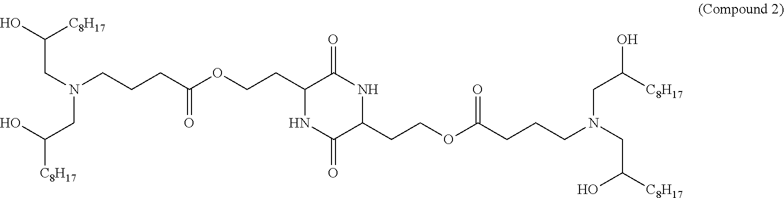

or a pharmaceutically acceptable salt thereof. In some embodiments, the compositions and methods of the present invention include a cationic lipid of the following formula:

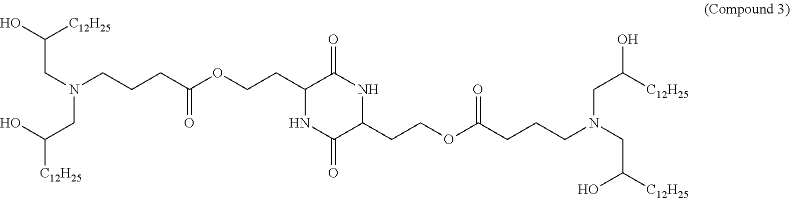

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

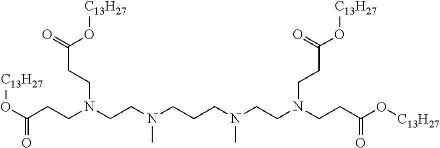

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

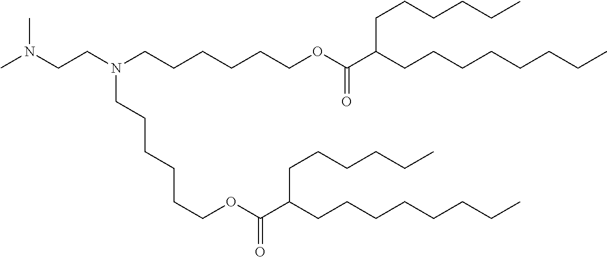 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

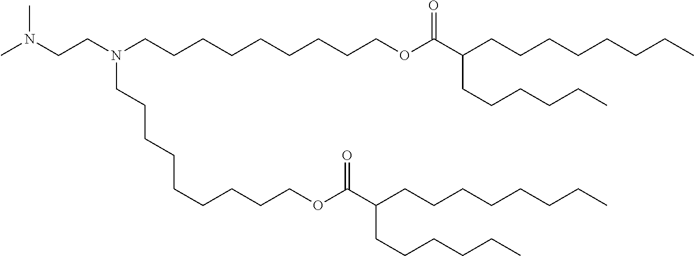 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

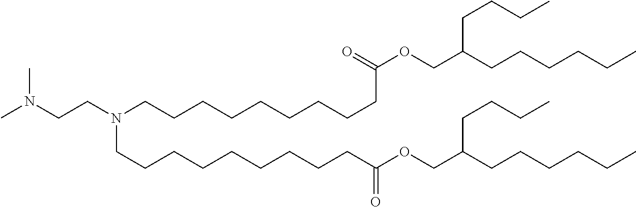 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

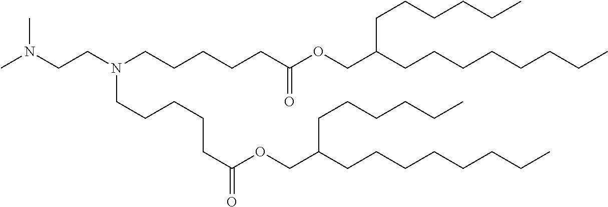 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

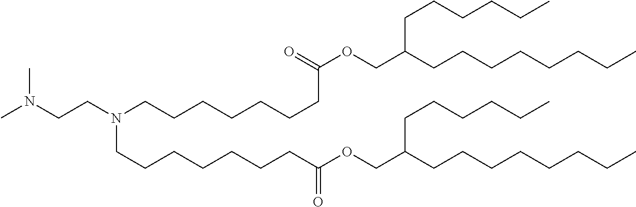 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

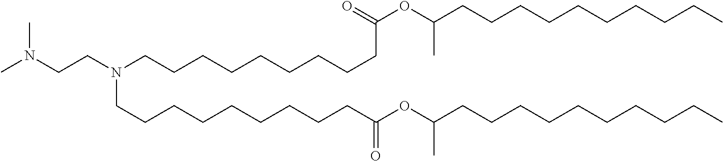 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

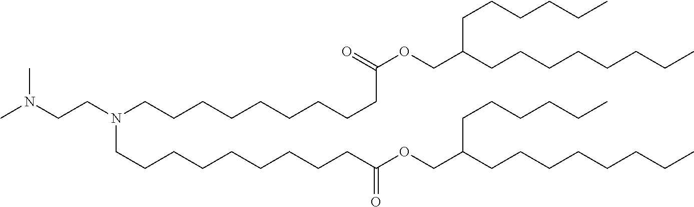 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

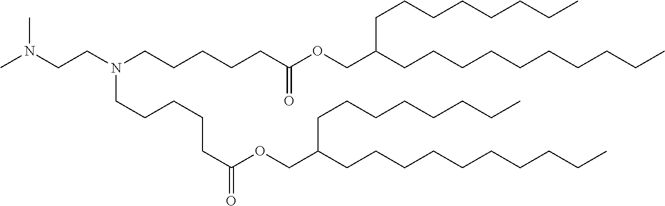 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

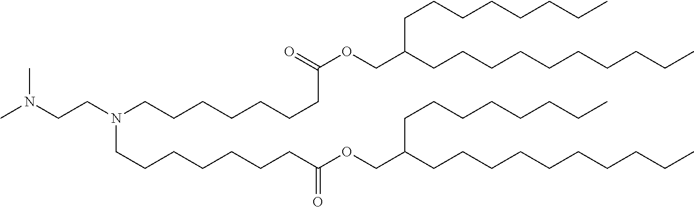 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

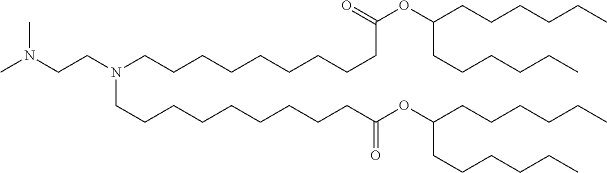 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

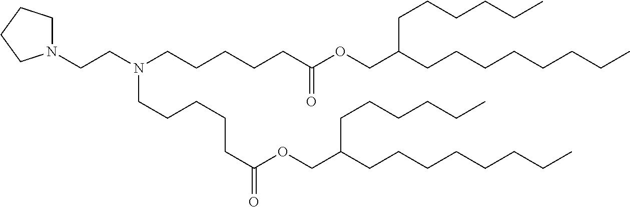 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

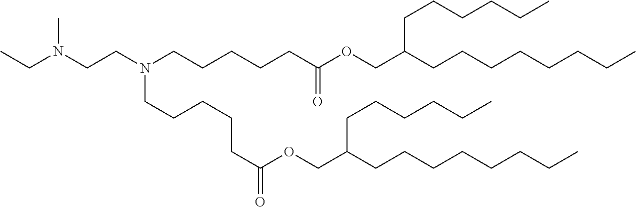 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

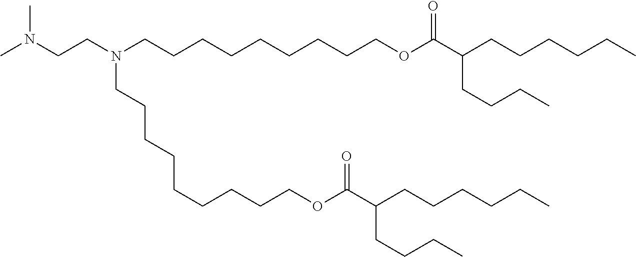 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

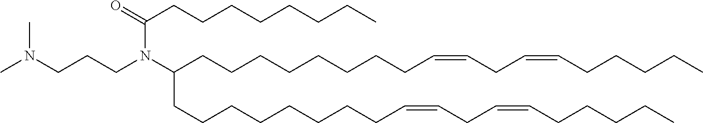 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

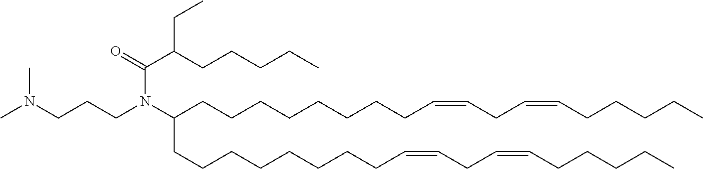 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

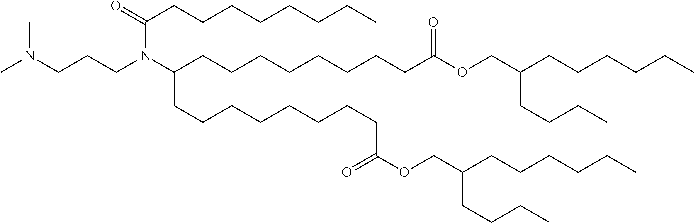 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

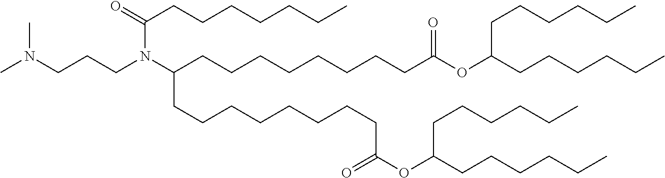 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

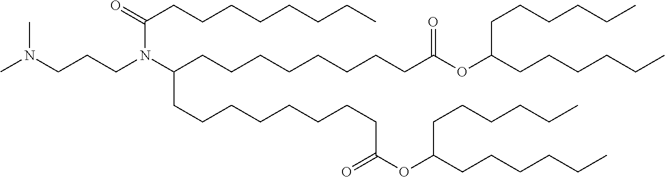 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

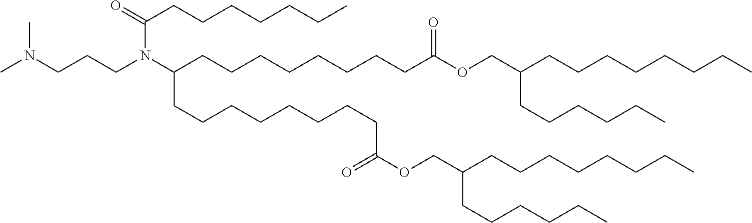 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

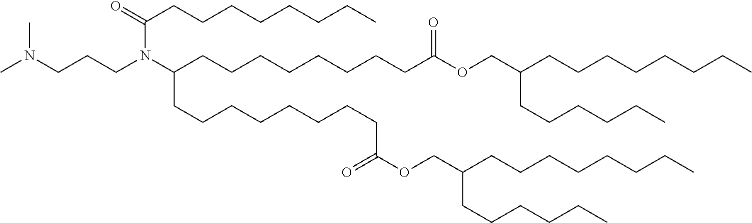 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

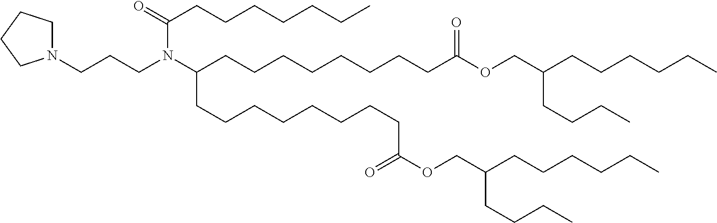 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

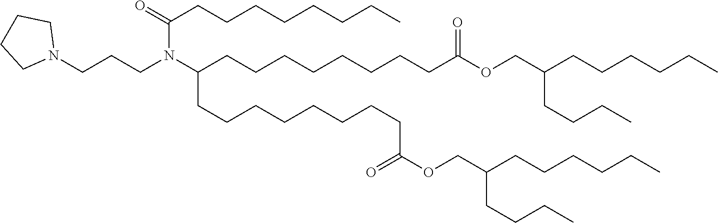 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

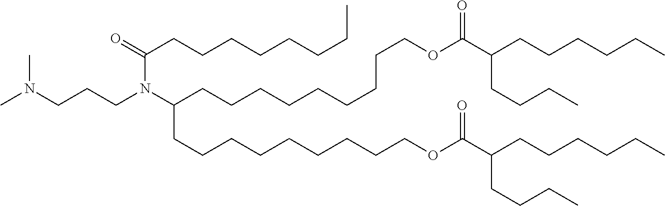 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

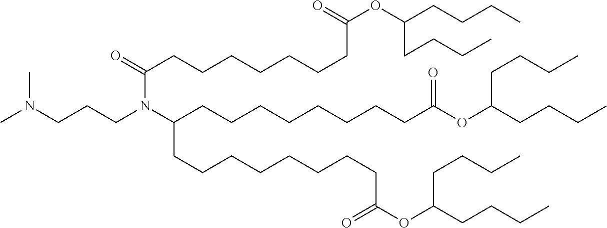 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

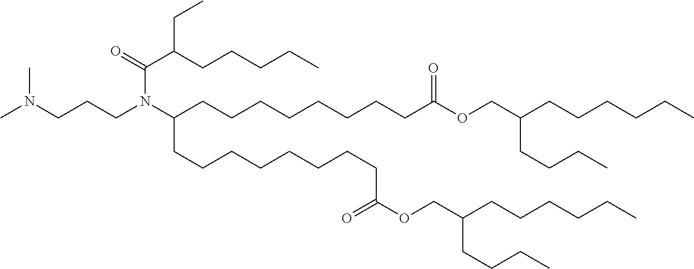 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

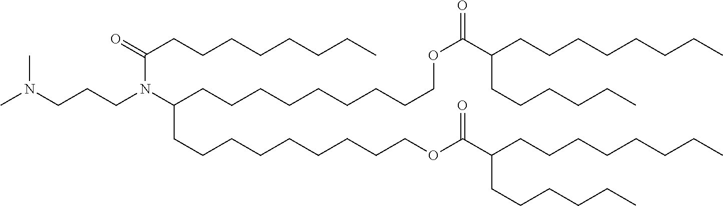 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

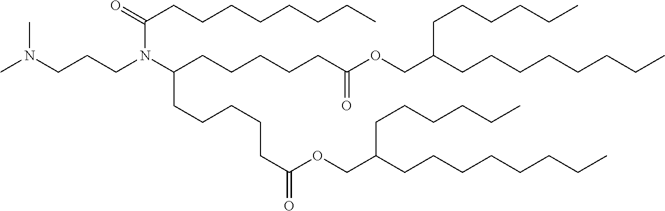 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

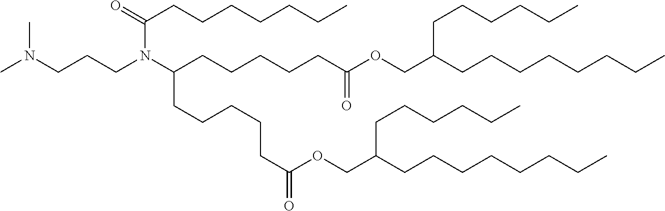 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

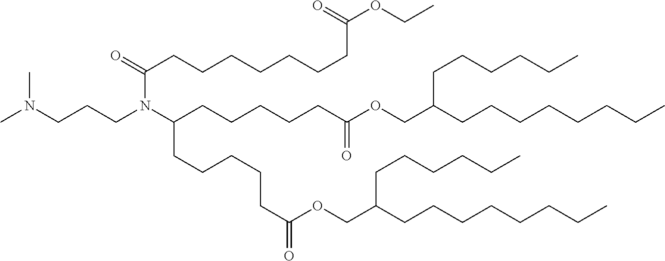 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

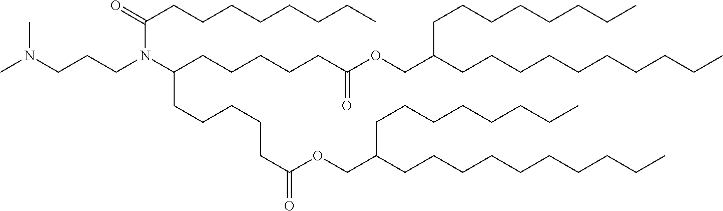 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

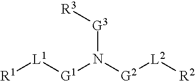

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂, alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵ C(═O)R⁴; R⁴ is C₁-C₁₂ alkyl; R⁵ is H or C₁-C₆ alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

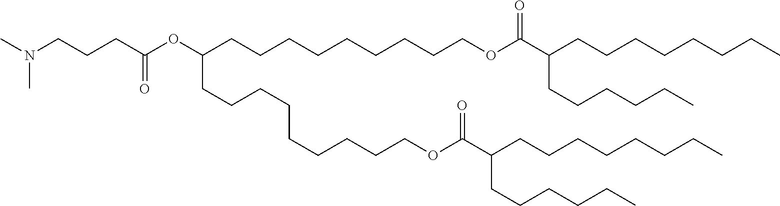 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

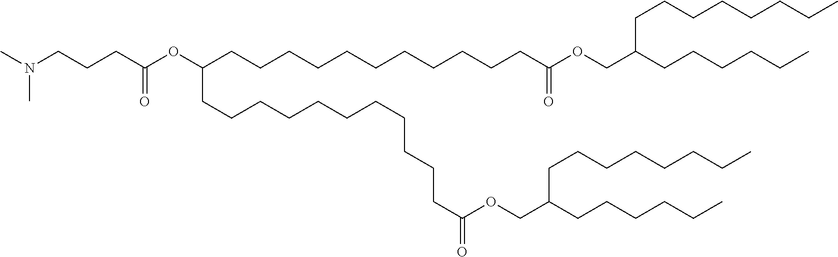 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

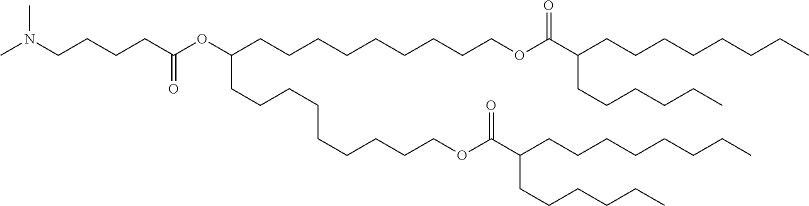 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

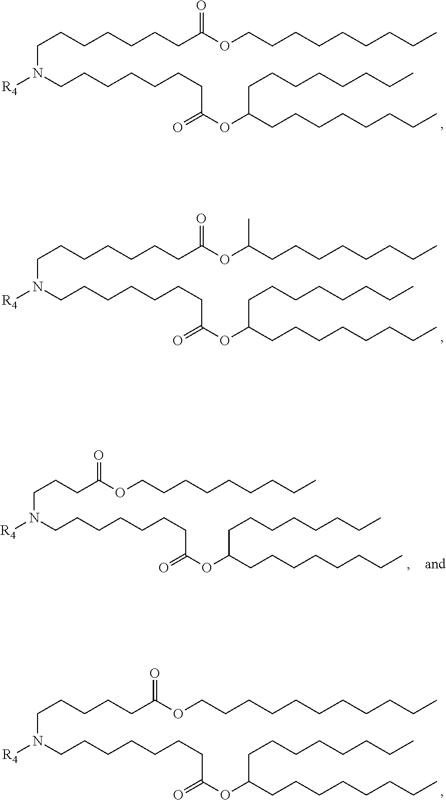

and pharmaceutically acceptable salts thereof. For any one of these four formulas, R₄ is independently selected from —(CH₂)-Q and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

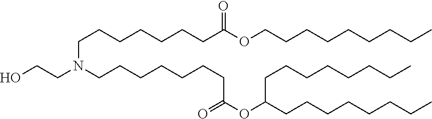 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

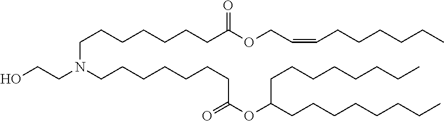 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

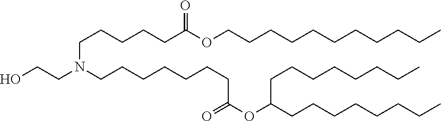 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

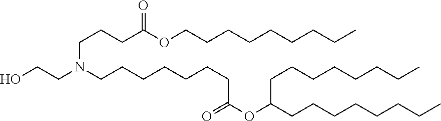 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of:

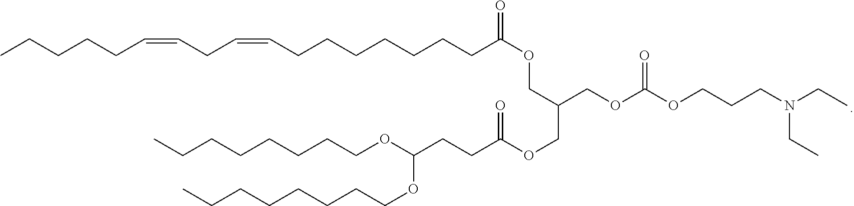 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In certain embodiments, the compositions and methods of the present invention include a cationic lipid having a compound structure of:

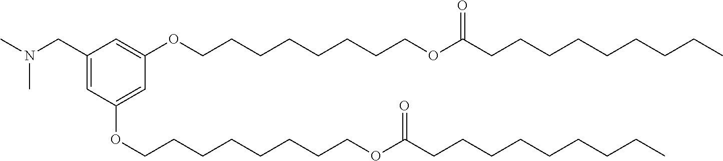 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

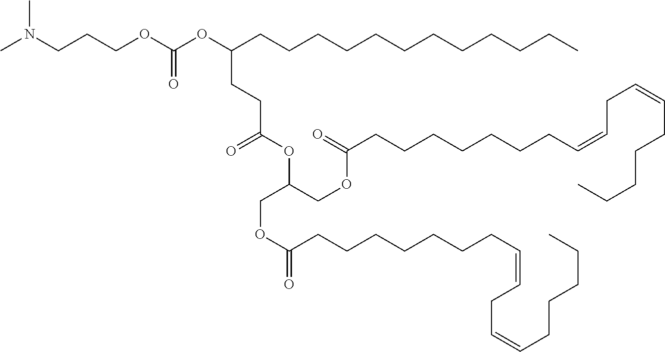 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

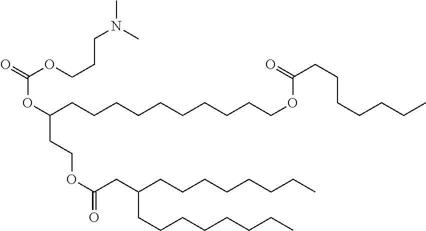 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

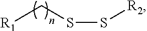

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

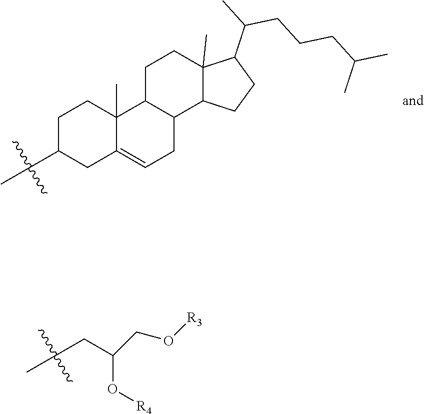

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

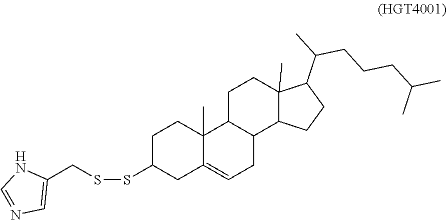 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002”, having a compound structure of:

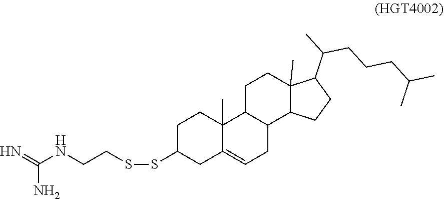 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003”, having a compound structure of:

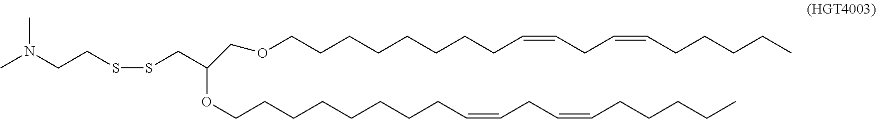 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004”, having a compound structure of:

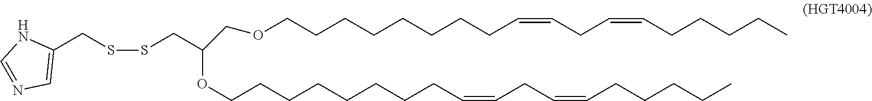 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005”, having a compound structure of:

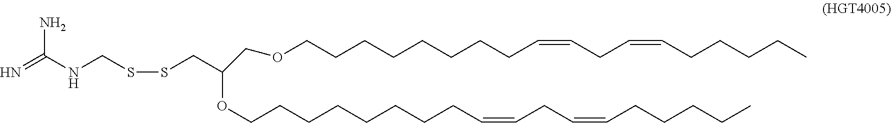 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present invention include cleavable cationic lipids as described in U.S. Provisional Application No. 62/672,194, filed May 16, 2018, and incorporated herein by reference. In certain embodiments, the compositions and methods of the present invention include a cationic lipid that is any of general formulas or any of structures (1a)-(21a) and (1b)-(21b) and (22)-(237) described in U.S. Provisional Application No. 62/672,194. In certain embodiments, the compositions and methods of the present invention include a cationic lipid that has a structure according to Formula (I′),

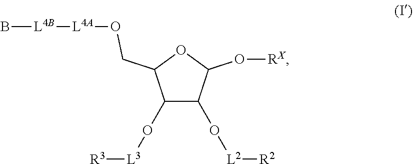

wherein:
R^{x} is independently —H, -L¹-R¹, or -L^{5A}-L^{5B}-B′;
each of L¹, L², and L³ is independently a covalent bond, —C(O)—, —C(O)O—, —C(O)S—, or —C(O)NR^{L}—;
each L^{4A} and L^{5A} is independently —C(O)—, —C(O)O—, or —C(O)NR^{L}—;
each L^{4B} and L^{5B} is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl; and
each R^{L} is independently hydrogen, C₁-C₂₀ alkyl, C₂-C₂₀ alkenyl, or C₂-C₂₀ alkynyl.
   In certain embodiments, the compositions and methods of the present invention include a cationic lipid that is Compound (139) of 62/672,194, having a compound structure of:

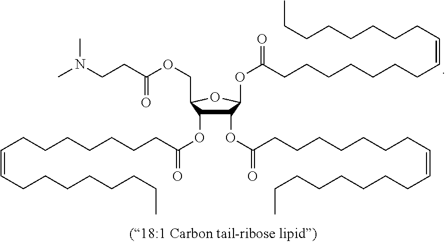

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.′1 Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′, 1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9, 12-dien-1-yloxy]propane-1-amine (“Octyl-CLinDMA”); (2R)-2-((8-[3beta)-cholest-5-en-3-yloxy]octyl)oxy)-N, N-dimethyl-3-[(9Z, 12Z)-octadeca-9, 12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present invention include 2,2-Dilinoleyl-4-dimethylaminoethyl-[1,3]-dioxolane (“XTC”); (3aR,5s,6aS)—N,N-dimethyl-2,2-dn(9Z,12Z)-octadeca-9,12-dienyetetrahydro-3aH-cyclopenta[d] [1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

Non-Cationic/Helper Lipids

In some embodiments, provided liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, such non-cationic lipids may be used alone, but are preferably used in combination with other lipids, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

Cholesterol-Based Lipids

In some embodiments, provided liposomes comprise one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm 179, 280 (1991); Wolf et al. BioTechniques 23, 139 (1997); U.S. Pat. No. 5,744,335), or ICE. In some embodiments, the cholesterol-based lipid may comprise a molar ration of about 2% to about 30%, or about 5% to about 20% of the total lipid present in a liposome. In some embodiments, the percentage of cholesterol-based lipid in the lipid nanoparticle may be greater than 5%, greater than 10%, greater than 20%, greater than 30%, or greater than 40%.

PEG-Modified Lipids

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle). Contemplated PEG-modified lipids include, but are not limited to, a polyethylene glycol chain of up to S kDa in length covalently attached to a lipid with alkyl chain(s) of C₆-C₂₀ length. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivitized lipids of the present invention may comprise a molar ratio from about 0% to about 20%, about 0.5% to about 20%, about 1% to about 15%, about 4% to about 10%, or about 2% of the total lipid present in the liposomal transfer vehicle.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the MCNA to be delivered. Additional considerations include, for example, the saturation of the alkyl chain, as well as the size, charge, pH, pKa, fusogenicity and toxicity of the selected lipid(s). Thus the molar ratios may be adjusted accordingly.

Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

A suitable liposome for the present invention may include one or more of any of the cationic lipids, non-cationic lipids, cholesterol lipids, PEG-modified lipids and/or polymers described herein at various ratios. As non-limiting examples, a suitable liposome formulation may include a combination selected from cKK-E12, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In various embodiments, cationic lipids (e.g., cKK-E12, C12-200, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C12-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

Ratio of Distinct Lipid Components

In embodiments where a lipid nanoparticle comprises three and no more than three distinct components of lipids, the ratio of total lipid content (i.e., the ratio of lipid component (1):lipid component (2):lipid component (3)) can be represented as x:y:z, wherein

(y+z)=100−x.

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a helper lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For compositions having three and only three distinct lipid components, variables “x,” “y,” and “z” may be in any combination so long as the total of the three variables sums to 100% of the total lipid content.

Formation of Liposomes Encapsulating mRNA

The liposomal transfer vehicles for use in the compositions of the invention can be prepared by various techniques which are presently known in the art. The liposomes for use in provided compositions can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA through electrostatic interactions. For example, during preparation of the compositions of the present invention, cationic liposomes may associate with the mRNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (zeta potential), targeting ligands and/or combinations thereof. In some embodiments, the one or more liposome may have a different composition of sterol-based cationic lipids, neutral lipid, PEG-modified lipid and/or combinations thereof. In some embodiments the one or more liposomes may have a different molar ratio of cholesterol-based cationic lipid, neutral lipid, and PEG-modified lipid used to create the liposome.

The process of incorporation of a desired mRNA into a liposome is often referred to as “loading”. Exemplary methods are described in Lasic, et al., FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of mRNA to the target cell or tissue.

Suitable liposomes in accordance with the present invention may be made in various sizes. In some embodiments, provided liposomes may be made smaller than previously known mRNA encapsulating liposomes. In some embodiments, decreased size of liposomes is associated with more efficient delivery of mRNA. Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-150 (1981), incorporated herein by reference. Average liposome diameter may be reduced by sonication of formed liposomes. Intermittent sonication cycles may be alternated with QELS assessment to guide efficient liposome synthesis.

### EXAMPLES

While certain compounds, compositions and methods of the present invention have been described with specificity in accordance with certain embodiments, the following examples serve only to illustrate the compounds of the invention and are not intended to limit the same.

### Example 1. Construct Design

Exemplary methods and designs of mRNA constructs for substrate-specific E3-ubiquitin ligase and variations of the same are provided in this example.

The basic design of an mRNA construct for substrate-specific E3-ubiquitin ligase comprises 1) a sequence encoding substrate binding domain and 2) a sequence encoding a fragment or full-length of E3 ubiquitin ligase. Optionally, a construct may further comprise a sequence encoding endoplasmic reticulum (ER) signal peptide, nuclear localization signal (NLS), and/or ER retention signal.

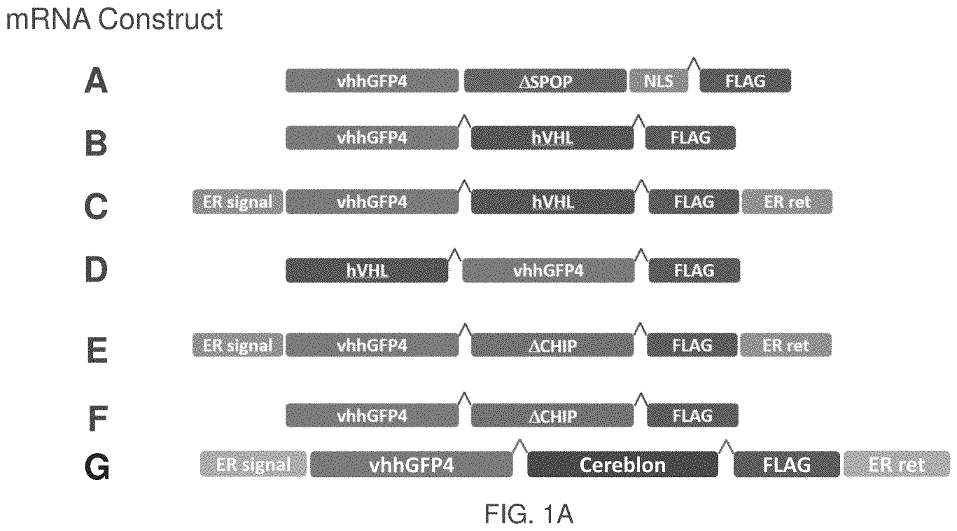

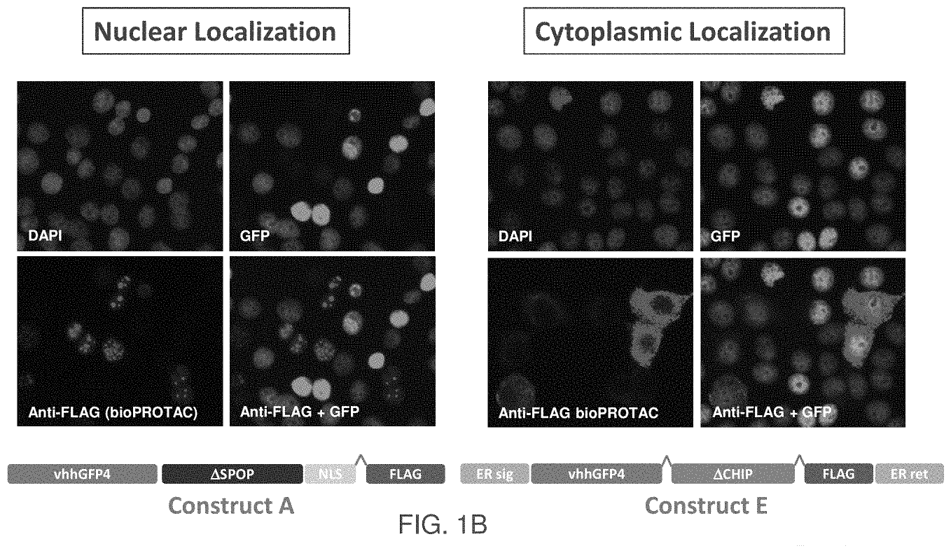

In this study, green fluorescent protein (GFP) was chosen as a target substrate. Various mRNA constructs were prepared as shown in FIG. 1A. vhhGFP4, a nanobody that specifically recognizes GFP, was used as a substrate binding domain. In each construct, vhhGFP4 was fused to an E3 ligase (ΔSPOP, hVHL, or ΔCHIP) with or without a flexible linker (as indicated by \hat{ } in FIG. 1A). Each construct was tagged with FLAG, which enables visualization with the anti-FLAG Cy3 dye. Constructs C and E further comprises sequences encoding ER signal peptide and ER retention signal. Components of each construct are shown in Table 1. Any number of variations of the above construct can be performed. For example, a linker can be modified, more than one E3 ligase may be used, or a sequence encoding E2 ubiquitin-conjugating enzyme can be introduced. Additionally, different combinations of substrate binding domain, E3 ligase, ER signal peptide, and ER retention signal can be contemplated.

The construct designs allow for specific subcellular targeting of proteins of interest. For example, degrading target proteins in some subcellular compartments maybe toxic. In order to avoid toxicity, targeting of a protein of interest can be restricted to specific subcellular compartments using the mRNA constructs provided herewith. Furthermore, using subcellular targeting signals is advantageous over other treatment strategies such as use of small molecules. Exemplary subcellular localization using the constructs described herein is shown in FIG. 1B. As seen in FIG. 1B, use of construct A provides for precise nuclear localization of the PROTAC, whereas use of a construct E provides for cytoplasmic localization of the PROTAC.

TABLE 1
mRNA Construct components

| | | Substrate | | | | ER | |
|---|---|---|---|---|---|---|---|
| | ER | binding | E3 | | | retention | |
| Construct | Signal | domain | Ligase | NLS | FLAG | signal | Comprises |
| A | no | vHHGFP4 | ΔSPOP | yes | yes | no | SEQ ID NO: 1 |
| B | no | vHHGFP4 | hVHL | no | yes | no | SEQ ID NO: 2 |
| C | yes | vHHGFP4 | hVHL | no | yes | yes | SEQ ID NO: 2, |
| | | | | | | | SEQ ID NO: 4-5 |
| D | no | vHHGFP4 | hVHL | no | yes | no | SEQ ID NO: 2 |
| E | yes | vHHGFP4 | ΔCHIP | no | yes | yes | SEQ ID NO: 3, |
| | | | | | | | SEQ ID NO: 4-5 |
| F | no | vHHGFP4 | ΔCHIP | no | yes | no | SEQ ID NO: 3 |
| G | Yes | vhhGFP4 | cereblon | no | yes | yes | SEQ ID Nos: 4-5 |

### Example 2. In Vitro Expression and Efficacy of mRNAs for Substrate-Specific E3-Ubiquitin Ligase Proteolysis

This example illustrates successful in vitro transfection, expression, and efficacy of mRNAs encoding substrate-specific E3-ubiquitin ligases.

GFP-expressing HeLa cells were transfected by mRNAs of constructs A, C, D, E, and F. After 24 hours of transfection, the untreated and transfected cells were stained and imaged using a microscope.

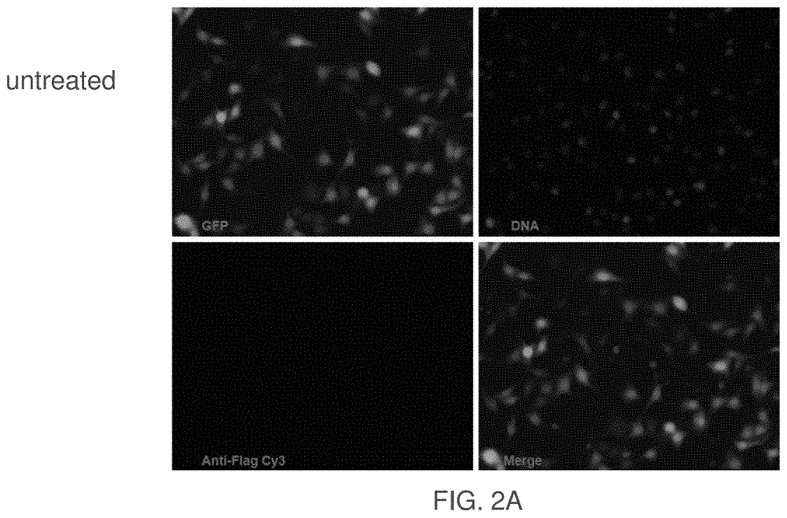

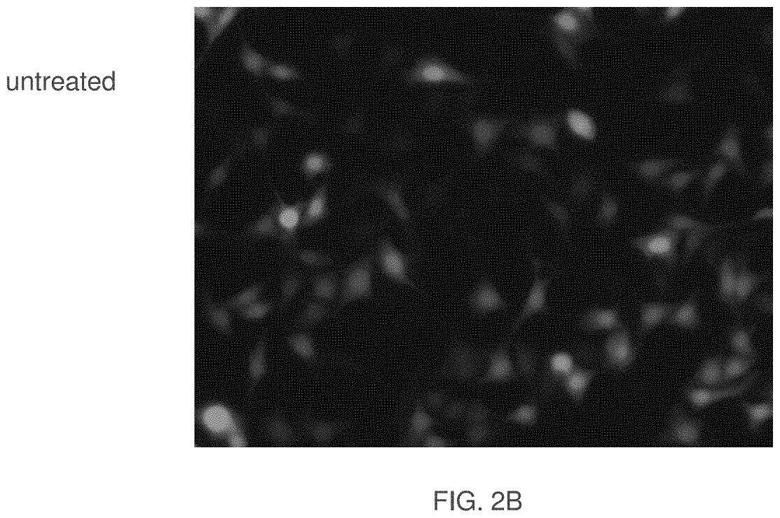

As depicted in FIG. 2A, the expressed GFP proteins and DNA were visualized by immunofluorescence. In the untreated cells, no signal was observed for anti-FLAG Cy3. A magnified merge image of GFP and FLAG signals for the untreated cells is shown in FIG. 2B.

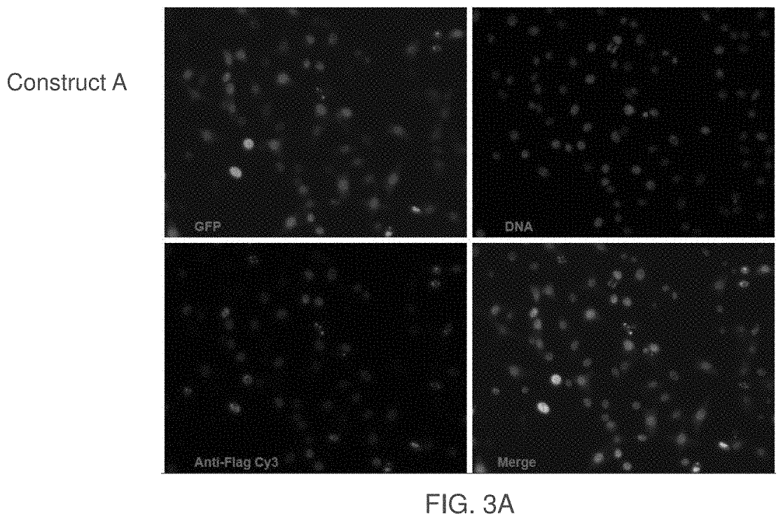

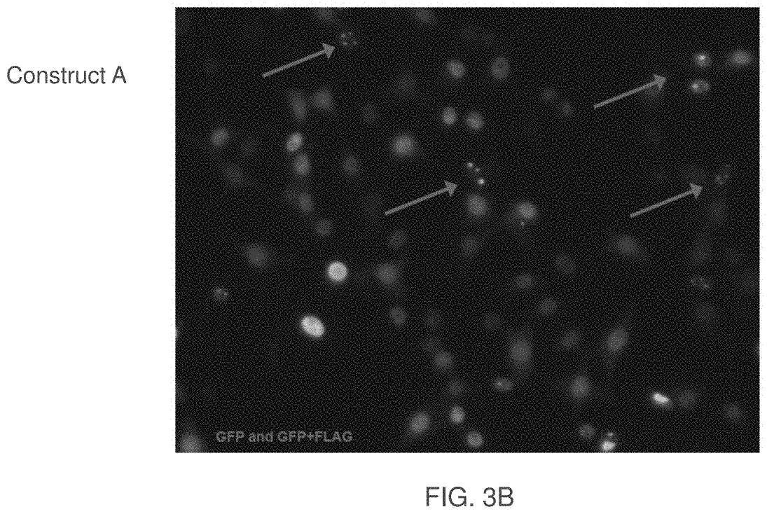

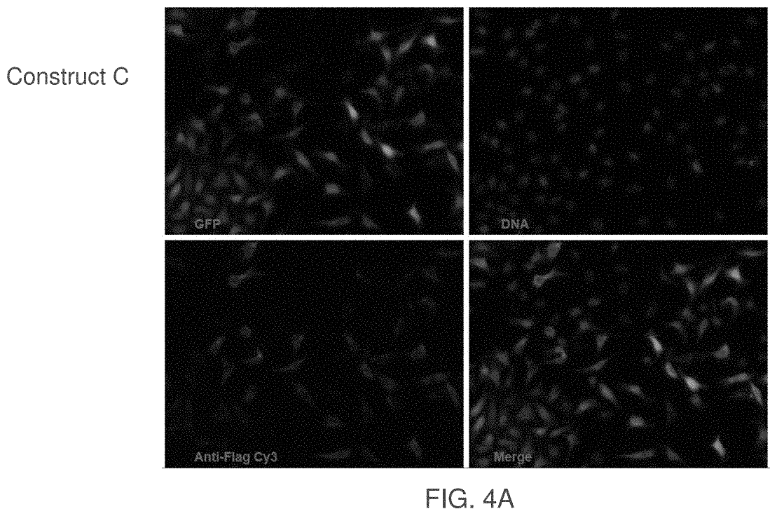

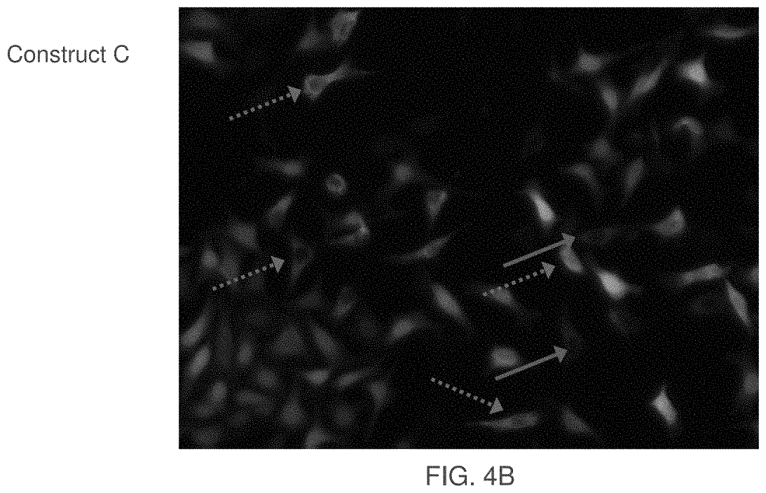

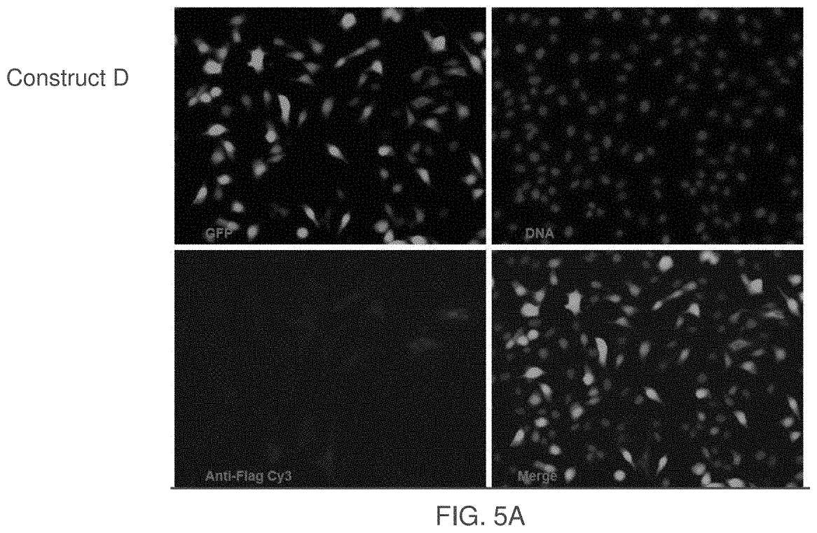

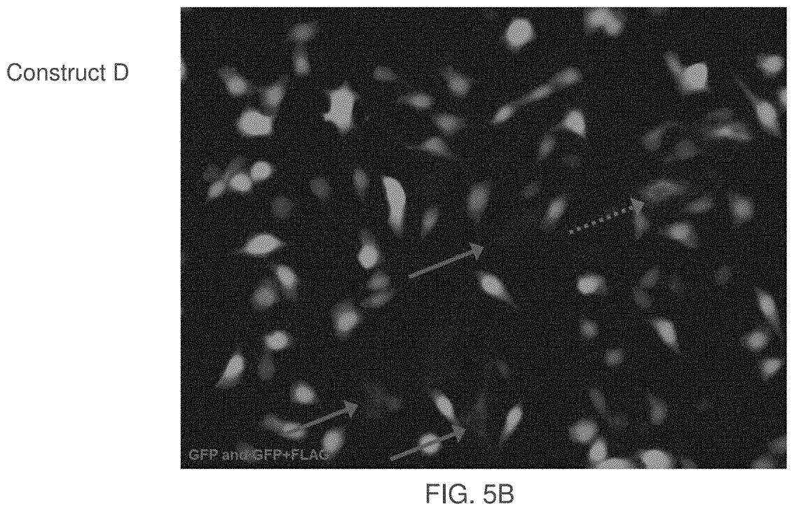

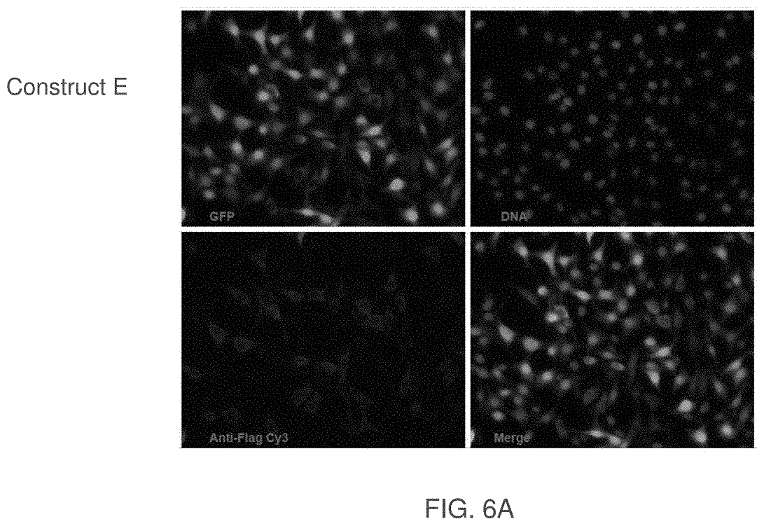

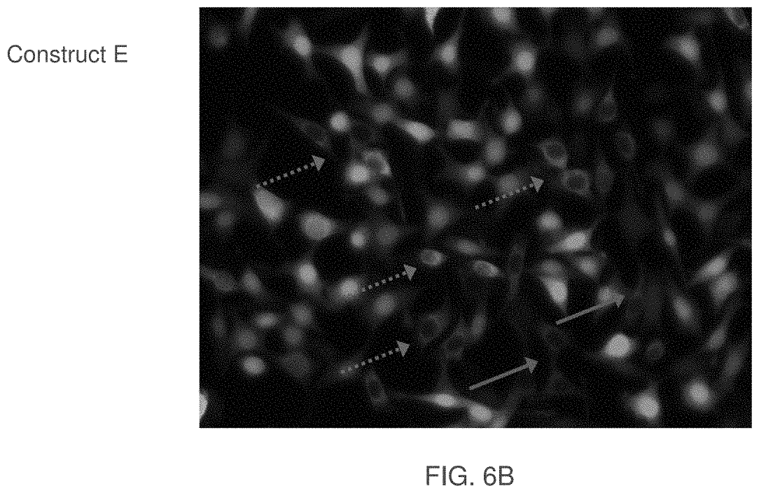

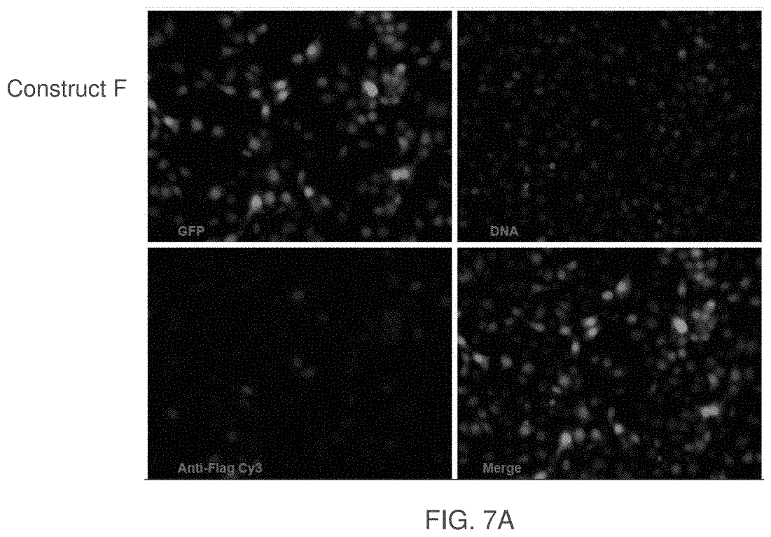

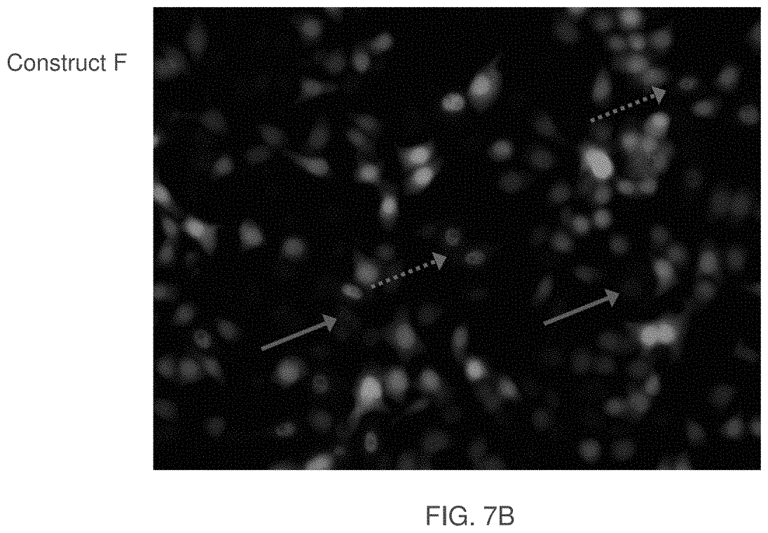

As shown in FIG. 3A-7B, cells transfected with various mRNA constructs shown in Table 1 successfully expressed the substrate-specific E3-ubiquitin ligases. Notably, as shown in merge images, FIG. 3B, FIG. 4B, FIG. 5B, FIG. 6B, and FIG. 7B, the expressed E3-ubiquitin ligases co-localized with GFP, indicating that the expressed E3-ligases were able to bind to their target, GFP.

Cells transfected with construct A mRNAs, which do not comprise ER signal peptide or ER retention signal, exhibited nucleus-associated speckles (FIG. 3B). Without wishing to be bound by theory, this demonstrates that the GFP-specific E3-ubiquitin ligases encoded by construct A bound to GFP in the transfected cells and translocated the GFP into the nucleus due to the lack or ER retention signal peptide.

Cells transfected with construct C or E mRNAs, which include ER signal peptide and ER retention signal, are shown in FIG. 4B and FIG. 6B, respectively. Interestingly, in these transfected cells that show expression of both GFP and E3-ubiquitin ligase (dashed arrows), GFP is sequestered outside of the nucleus.

Cells transfected with construct F mRNAs, which do not comprise ER signal or ER retention signal peptide, are shown in FIG. 7B. Notably, in cells that showed expression and co-localization of GFP and E3-ubiquitin ligases, “holes” were visible in the nucleus, demonstrating the degradation of GFP mediated by ubiquitin degradation pathway (see blue arrows in FIG. 7B).

Overall, this example shows that cells transfected with various mRNA constructs successfully expressed the GFP-specific E3-ubiquitin ligases. In turn, these expressed GFP-specific E3-ubiquitin ligases bound GFP and induced selective proteolysis.

### Example 3. Time Course Study of Expression and Efficacy of mRNAs for Substrate-Specific E3-Ubiquitin Ligase Proteolysis

This example illustrates successful expression and efficacy of mRNAs encoding substrate-specific E3-ubiquitin ligases at 6 and 24 hours post-transfection.

HEK293 cells were transfected by mRNAs of constructs A or E, or GFP mRNA alone. Additionally, HEK293 cells were co-transfected with GFP mRNA and mRNA construct A or E. The cells were stained at 6 or 24 hours post-transfection and were imaged using a microscope at 40× magnification. The study design is shown in Table 2.

TABLE 2
Study design of induced selective
proteolysis in HEK193 cells

| | E3 Ligase mRNA | GFP | Post- |
|---|---|---|---|
| Sample | construct | mRNA | Transfection |
| 1 | — | — | 6 hours |
| 2 | — | yes | 6 hours |
| 3 | Construct A | — | 6 hours |
| 4 | Construct E | — | 6 hours |
| 5 | Construct A | yes | 6 hours |
| 6 | Construct E | yes | 6 hours |
| 7 | — | — | 24 hours |
| 8 | — | yes | 24 hours |
| 9 | Construct A | — | 24 hours |
| 10 | Construct E | — | 24 hours |
| 11 | Construct A | yes | 24 hours |
| 12 | Construct E | yes | 24 hours |

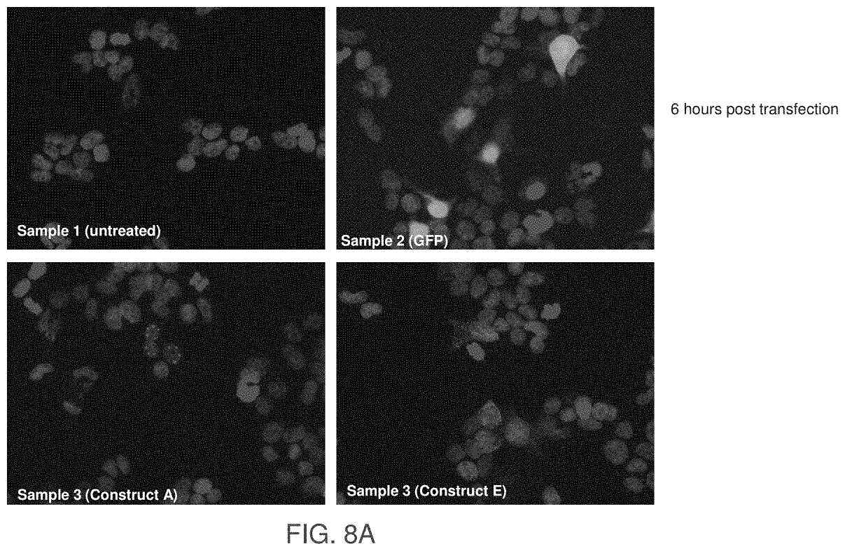

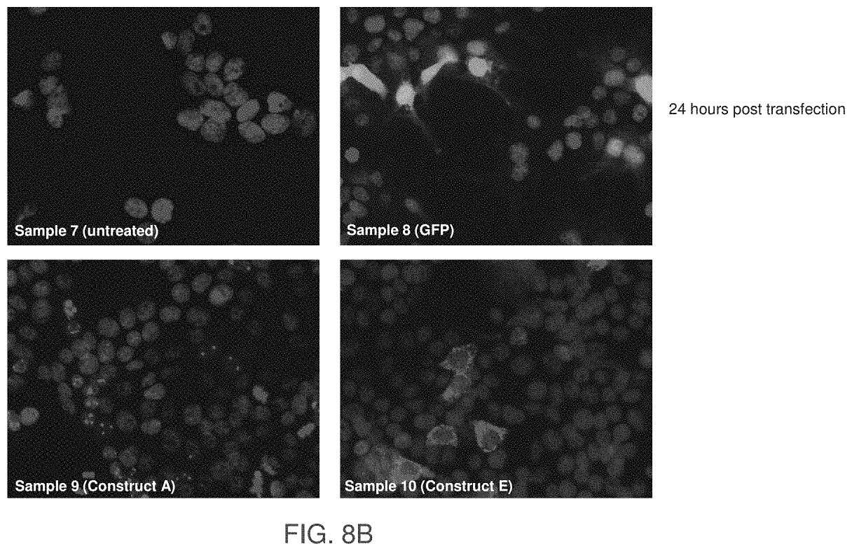

As shown in FIG. 8A, single-construction transfection of each mRNA (Samples 2-4 in Table 2) resulted in moderate expression of either GFP or E3 ligase as compared to the untreated sample 1, 6 hours after transfection. For sample 2, transfected GFP was present uniformly throughout the cell. For sample 2, transfected with construct A, which contains the NLS but not the ER signal peptide or ER retention signal, showed that the expressed E3 ligase localized to the nucleus in speckles. For sample 4, transfected with construct E, which includes ER signal peptide and ER retention signal, showed that the expressed E3 ligase remained in the cytoplasm.

As shown in FIG. 8B, each construct demonstrated an increased expression after 24 hours post-transfection (Samples 8-10). Localization of the expressed proteins was similar to what was observed at 6 hour post-transfection samples.

Next, HEK293 cells were co-transfected by GFP mRNA with construct A or E, as indicated for samples 5, 6, 11 and 12 in Table 2 and imaged at 6 or 24 hours post-transfection.

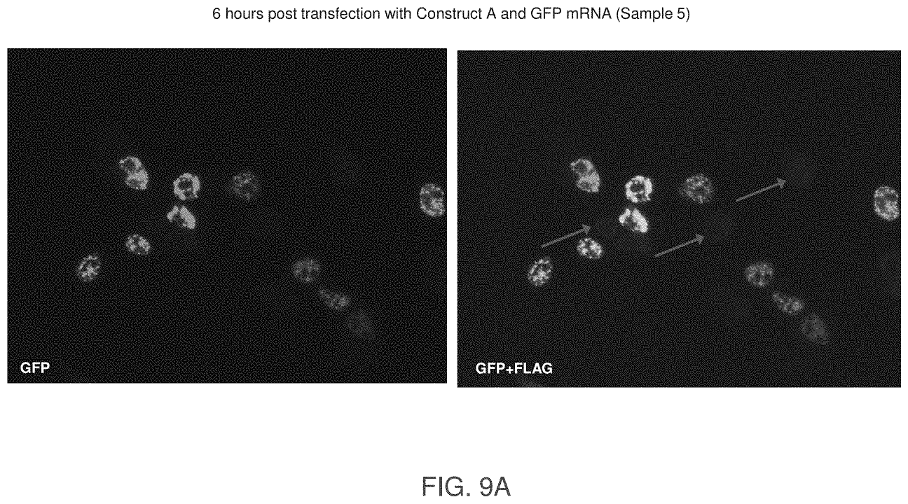

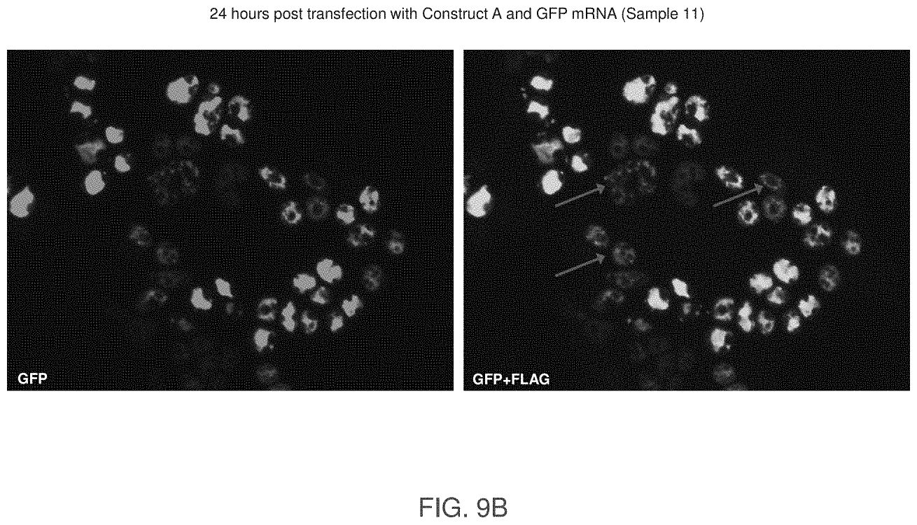

When cells were transfected by GFP mRNA alone, GFP was expressed throughout the cell as shown in FIG. 8A and FIG. 8B (samples 2 and 8). However, when the cells were co-transfected by GFP mRNA and construct A, which contains NLS, the expressed GFP was sequestered into the nucleus, indicating that the expressed E3 ligase is able to bind to GFP and move into the nucleus (FIG. 9A and FIG. 9B). Additionally, “holes” were visible in the nucleus, suggesting the degradation of GFP mediated by ubiquitin degradation pathway.

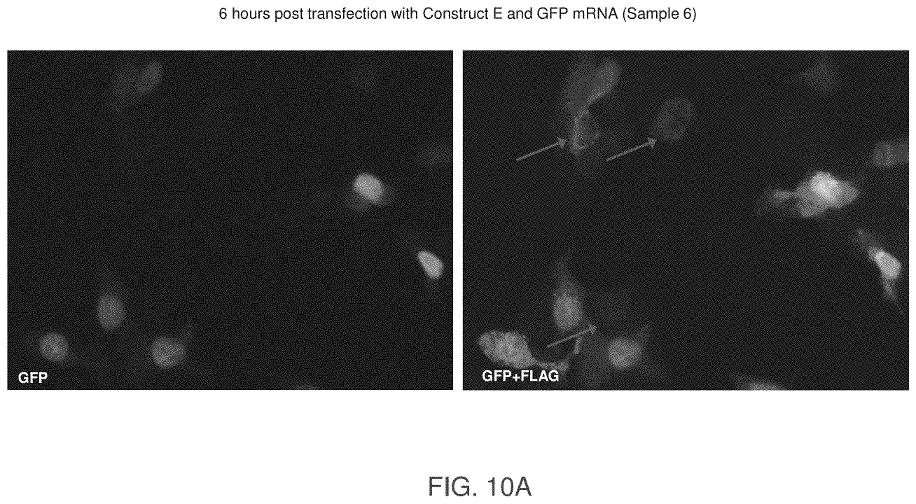

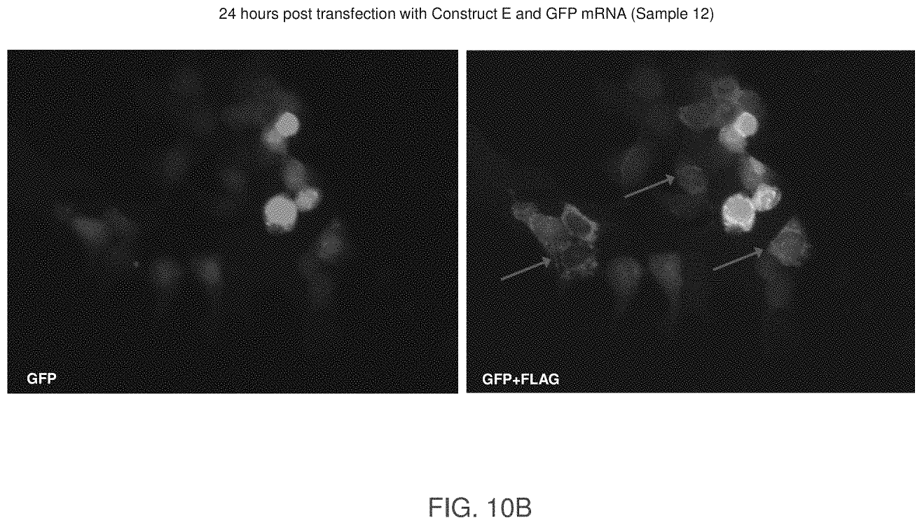

As illustrated in FIG. 10A and FIG. 10B, cells transfected with GFP mRNA and construct E (samples 6 and 12), show that cytoplasmic GFP signal was reduced in regions expressing E3 ligase, while the nuclear GFP signal remained, for both 6 and 24 hours post-transfection. This shows that E3 ligase expressed by construct E degraded cytoplasmic GFP. At 24 hours post-transfection, nuclear GFP appeared to be slightly reduced, suggesting that the E3 ligase might be degrading GFP before moving out of the nucleus, reducing the nucleus.

Overall, this example shows that E3 ligase expressed by the transfected mRNAs successfully bound GFP and induced selective proteolysis. This example further demonstrates that the E3-ubiquitin ligase induced proteolysis of the present invention can be made specific to a subcellular compartment.

### Example 4. In Vitro Efficacy of E3-Ubiquitin Ligase Induced Proteolysis of Nucleus GFP

This example illustrates that the expressed E3-ubiquitin ligases are able to bind its target substrate in the nucleus and induce proteolysis.

HeLa cell line stably expressing GFP, which has been modified with a Histone H2B tag, was transfected by construct A or E. Histone H2B is one of the four main histone proteins that form the nucleosome, and thus, the H2B-tagged GFP is localized exclusively in the nucleus. Additionally, the H2B tag is also thought to slightly alter the conformation of GFP, potentially making it more amendable to poly-ubiquitination or proteasomal degradation.

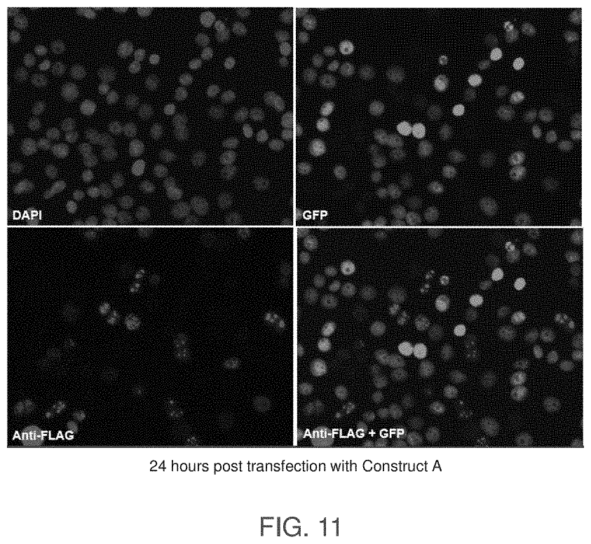

The transfected cells were stained at 24 hours post-transfection and were imaged using a microscope at 40× magnification. FIG. 11 shows images of cells transfected with construct A and H2B-tagged GFP mRNA. As shown in the upper right panel, GFP was exclusively localized to the nucleus due to the H2B tag. Additionally, E3 ligase encoded by construct A localized in nuclear speckles as seen previously and on lower left panel. Interestingly, as shown in lower right panel of FIG. 11, E3 ligase did not reveal any co-localization with GFP, suggesting that the H2B-tagged GFP is being degraded efficiently in the nucleus.

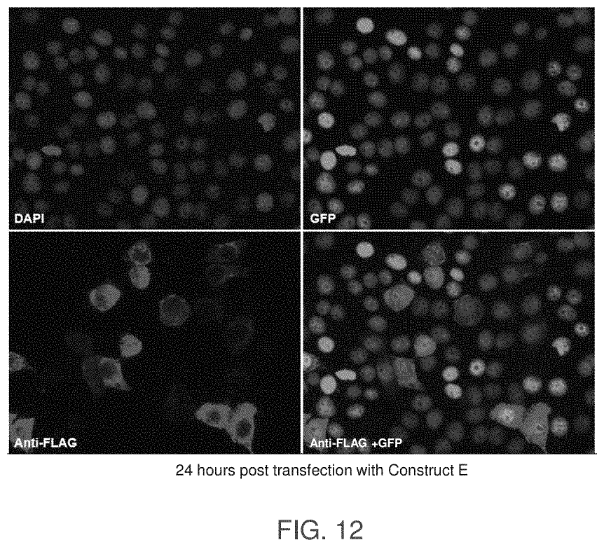

FIG. 12 shows the stained images of cells transfected with construct E and H2B-tagged GFP mRNA. Similar to FIG. 11, it shows that the GFP was localized to the nucleus. Since construct E contains ER signal peptide and ER retention signal, the E3 ligase encoded by the transfected mRNA localized in the cytoplasm, as shown in lower left panel of FIG. 12. In contrast to FIG. 11, the merge image on lower right panel shows that nuclear GFP was clearly present in cells that expressed E3 ligase (FIG. 12, lower right panel). As H2B-tagged GFP was restricted to the nucleus, GFP could not be degraded by E3-ligase inducted proteolysis pathway.

### Example 5. Concentration-Dependent Response of E3-Ubiquitin Ligase Induced Proteolysis of GFP

This example illustrates that proteolysis induced by the expressed E3-ubiquitin ligases is concentration-dependent.

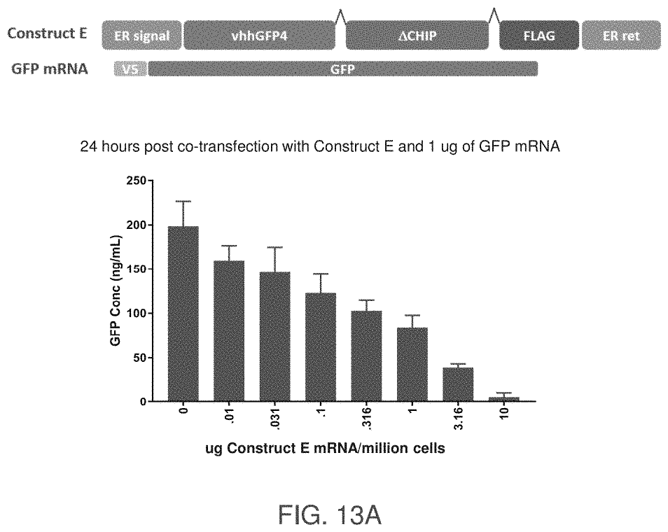

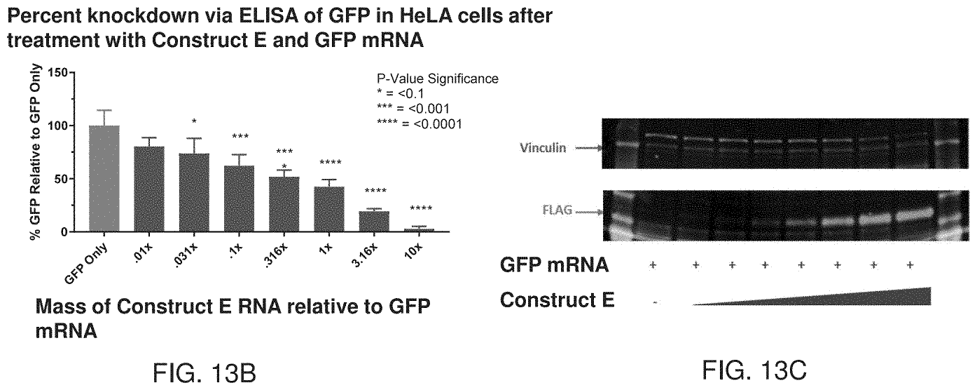

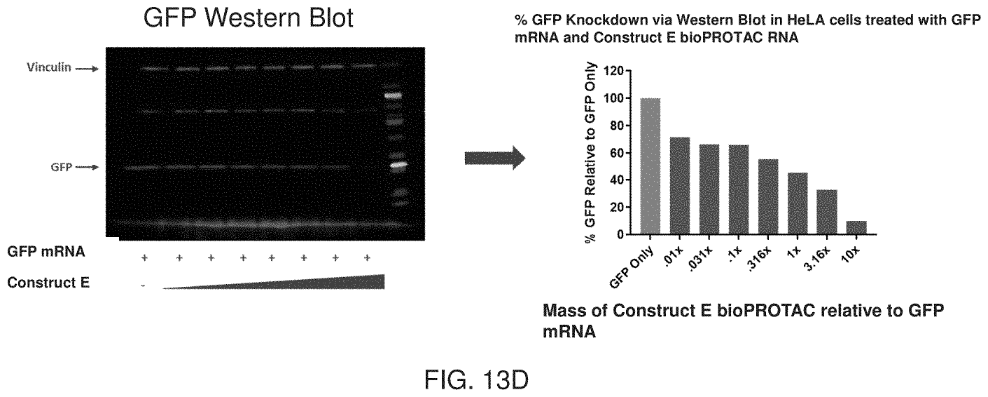

HeLa cell line that does not endogenously express GFP was co-transfected with 1 μg of GFP mRNA and a varying concentration of construct E. The co-transfected cells were stained at 24 hours post-transfection and were imaged. The amount of GFP was quantified and plotted as shown in FIG. 13A-B, D. FIG. 13C is a FLAG Western blot that shows construct E reduced GFP expression in a concentration-dependent manner FIG. 13D is a GFP Western Blot that shows construct E reduced GFP expression in a concentration-dependent manner Overall, the results show that E3-ubiquitin ligase encoded by the construct E mRNA efficiently induced degradation of GFP in a concentration-dependent manner.

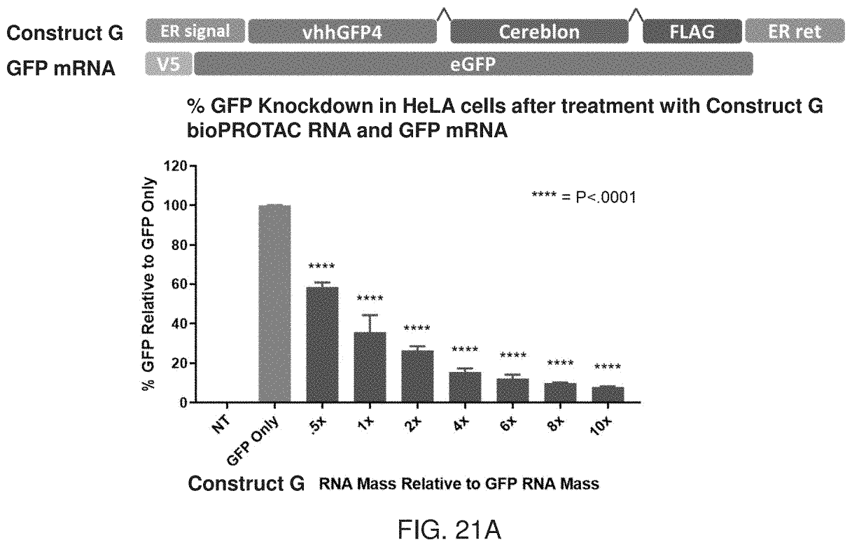

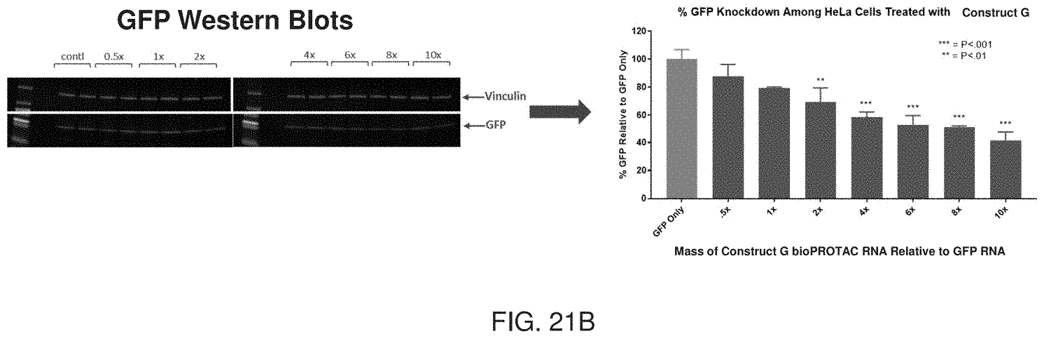

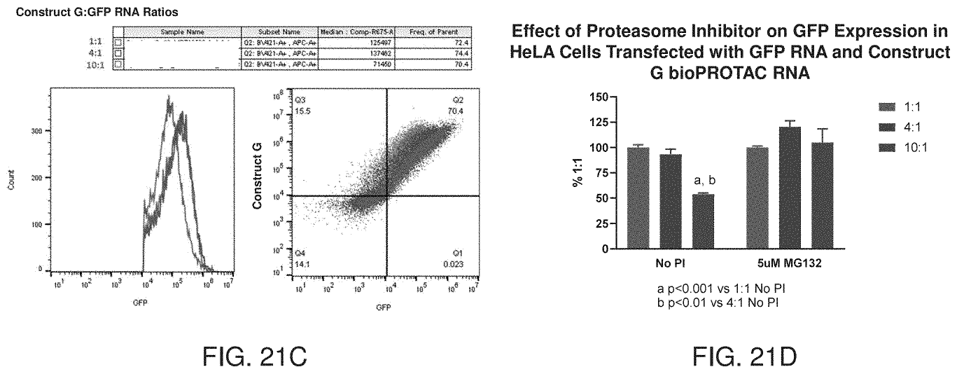

Another E3-ubiquitin ligase was tested and shown to provide targeted proteolysis in a concentration-dependent manner. This ubiquitin construct, construct G, contains the E3 ligase cereblon, ER signal, ER retention sequence, and vhhGFP. The data from this study showed that construct G reduced GFP expression in a concentration-dependent manner (FIG. 21A-B). The study design was as described in the paragraph above. Additional data was generated using Construct G which showed a concentration-dependent response of Construct G on GFP expression. These data are presented in FIG. 21C, which shows a flow cytometry plot with HeLA cells that had been exposed to Construct G:GFP RNA ratios of 1:1, 4:1, and 10:1. These data are shown as bar graphs in FIG. 21D. Overall, these data showed a concentration-dependent decrease in the amount of GFP as the ratio of Construct G increased. Specifically, the data showed a 46% decrease in GFP mean fluorescence intensity (MFI) at 10:1 Construct G:GFP RNA that is blocked with 5 μM MG132.

### Example 6. Time-Course Study of E3-Ubiquitin Ligase Induced Proteolysis of GFP

This example studies time-course degradation of GFP induced by E3-ubiquitin ligase encoded by the administered mRNA.

HeLa cell line that does not endogenously express GFP was co-transfected with GFP mRNA and construct E. The amount of GFP in the co-transfected cells were measured at various time points, up to 34 hours post-transfection. As a negative control, HeLa cell line that does not endogenously express GFP, which was transfected with GFP mRNA alone, was also measured for GFP concentration.

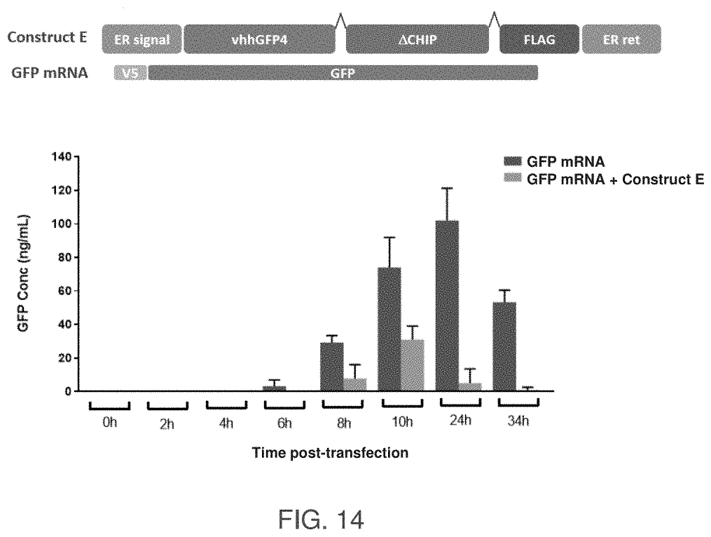

The amount of GFP at various time points were plotted in FIG. 14. As compared to the GFP level in cells transfected with only GFP mRNA, the GFP level in cells co-transfected with GFP mRNA and construct E encoding E3-ubiquitin ligase was significantly decreased at all time-points. The results also show that E3-ubiquitin ligase encoded by the administered mRNA is effective as soon as 6 hours after transfection (as soon as the GFP expression was detectable), and the effect lasts even after 34 hours post-transfection.

Next, HeLa cell line stably expressing GFP, which has been modified with a Histone H2B tag, was transfected by construct A. Construct A has a nucleus localize signal, therefore induces the expression of E3-ubiquitin ligase in the nucleus. The amount of GFP in the transfected cells were measured at various time points, up to 72 hours post-transfection. As a negative control, HeLa cell line that constitutively expresses H2B-GFP, which was not transfected with construct A, was also measured for GFP concentration.

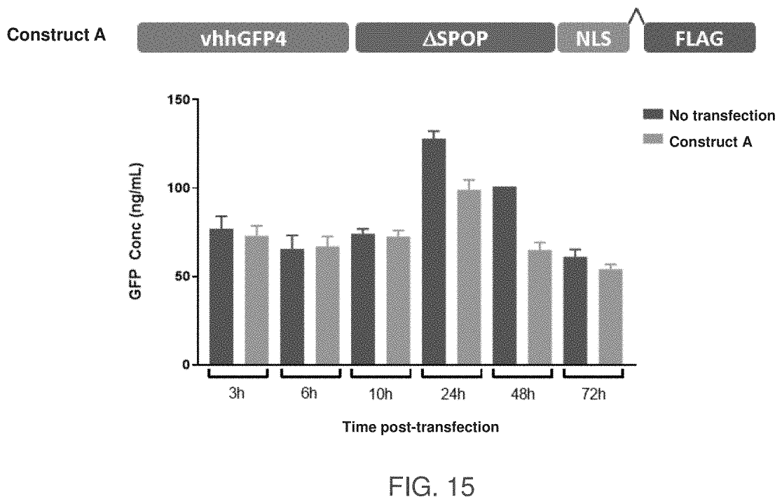

The amount of GFP at various time points were plotted in FIG. 15. There was no significant change in GFP level before 10 hours post-transfection as compared to the negative control. At 24 and 48 hour time-points, an apparent reduction in GFP concentration was observed as compared to the negative control.

### Example 7. In Vitro Efficacy of E3-Ubiquitin Ligase Induced Proteolysis of GFP in Cell-Free System

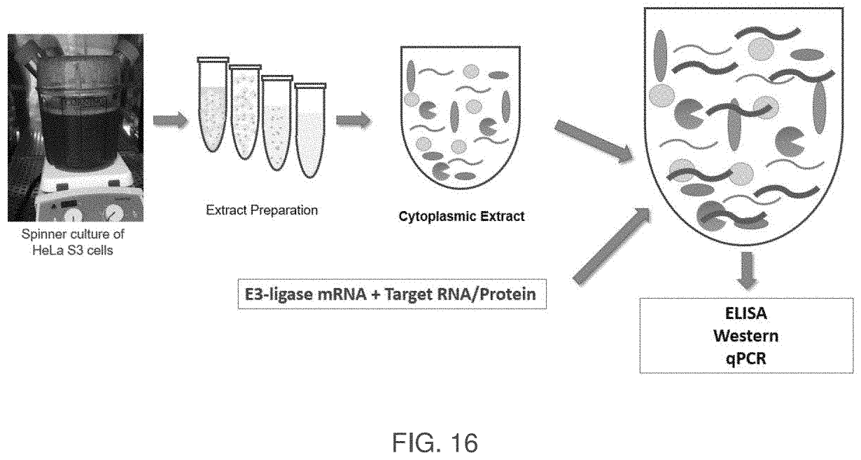

This example studies E3-ubiquitin ligase induced proteolysis of GFP in vitro translation system (cell-free system). The study design is depicted in FIG. 16. Briefly, cytoplasmic extracts of HeLa cells were prepared according to methods known in art. To cytoplasmic extracts, which contain functional translation system, E3-ligase mRNA and target mRNA or protein were added. Amount of mRNA or GFP were quantified by ELISA, Western blot, or qPCR.

The efficacy GFP degradation induced by administered mRNA encoding E3-ubiquitin ligase in the in vitro cell-free system was examined Cytoplasmic extracts were added with various components at different ratios as shown in Table 3.

TABLE 3
In Vitro Translation System with GFP mRNA

| | | Construct E | |
|---|---|---|---|
| | | (GFP mRNA: | |
| Sample | GFP mRNA | Construct E) | Non-anti-GFP |
| 1 | 5 pmol | — | — |
| 2 | 5 pmol | 2:1 | — |
| 3 | 5 pmol | 1:1 | — |
| 4 | 5 pmol | 1:2 | — |
| 5 | 5 pmol | 1:2 | 2.5 pmol |
| 6 | 0 pmol | — | — |

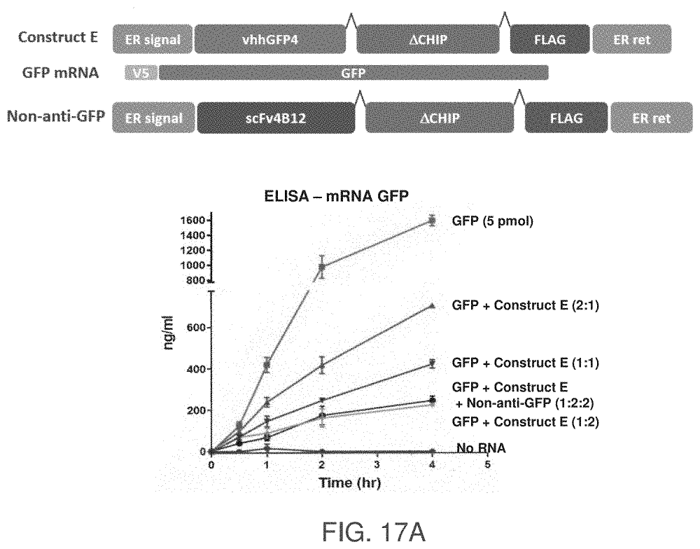

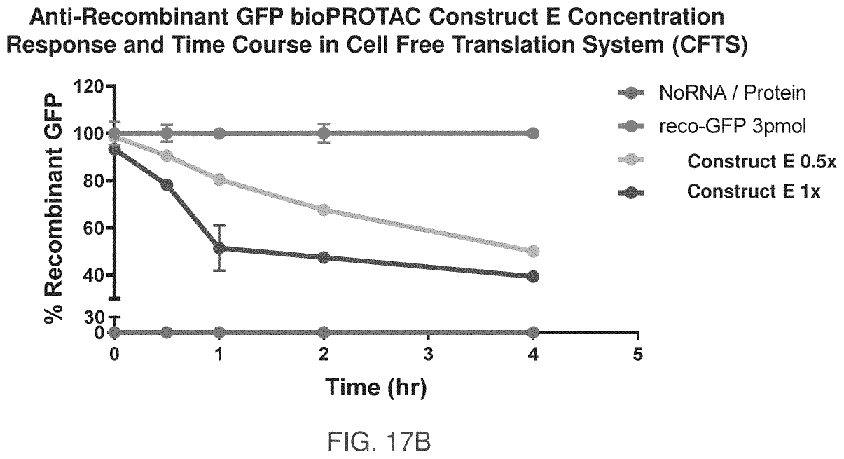

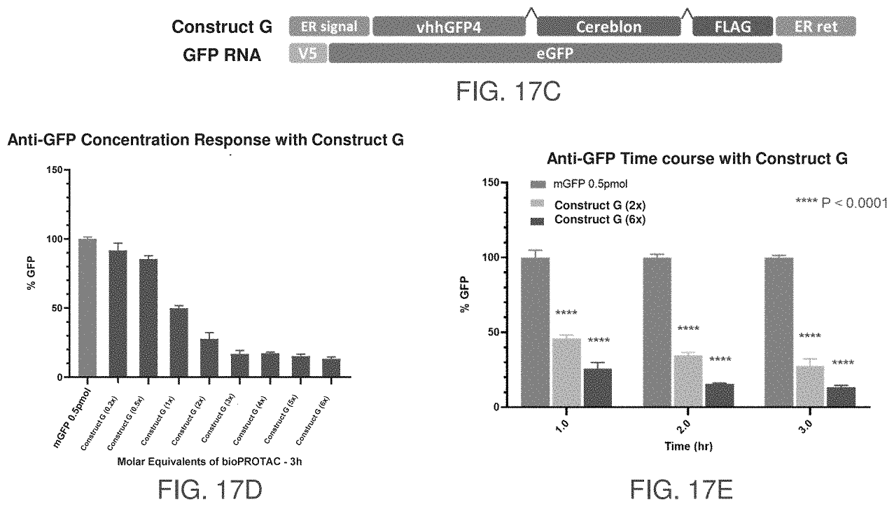

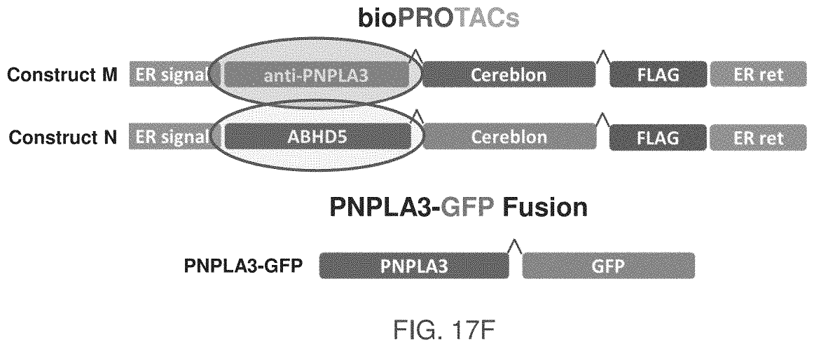

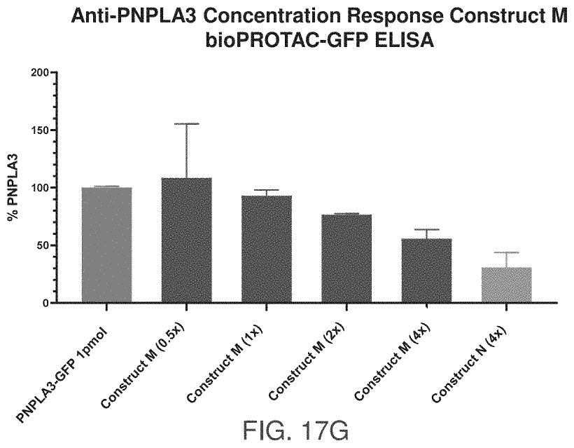

As shown in FIG. 17, sample containing GFP mRNA alone (Sample 1) produced significantly high amount of GFP protein, whereas sample without any mRNA added (Sample 6) contained undetectable amount of GFP. Samples 2-4, which were supplemented with varying amount of construct E showed dose-dependent reduction in GFP, illustrating that E3-ubiquitin ligase encoded by construct E successfully induced GFP proteolysis. To examine if there is limitation in translation of GFP and/or E3-ubiquitin ligase, an mRNA encoding E3-ubiquitin ligase targeting non-GFP was added (Sample 5). The results show there was no significant difference between GFP in sample 4 as compared to sample 5, demonstrating that production of GFP and/or E3-ubiquitin ligase is not limited by the translation efficiency. FIG. 17B presents data that shows degradation of recombinant GFP using Construct E in a cell-free translation system (CFTS). Data from this study showed that bioPROTAC activity was observed after 30 minutes in the CFTS.

A cell free translation system (CFTS) was also used to assess anti-GFP bioPROTAC using the E3-ligase cereblon, Construct G (FIGS. 17C-E). FIG. 17C is a schematic showing Construct G and a construct comprising GFP RNA. These CFTS studies showed both an anti-GFP concentration response with Construct G (FIG. 17D), and a progressive reduction of anti-GFP over a three-hour time course assessment (FIG. 17E). The total RNA/sample was 3.5 pmol. The data showed significant GFP knockdown using Construct E even at 0.2 eq.

Another CFTS study was performed using a bioPROTAC E3 ligase comprising cereblon and either an anti-PNPLA3 scFv antibody (Construct or ABHD5 (FIG. 17F). ABHD5 is a PNPLA3 protein binder. FIG. 17F is a schematic which shows the cereblon comprising E3 ligase bioPROTACs and also shows a PNPLA3-GFP fusion. For these studies, PNPLA3-GFP fusion constructs and/or the constructs M or N were used in the CFTS system. The data showed a concentration dependent decrease in the amount of PNPLA3-GFP with increasing amounts of construct M or N (FIG. 17G). These data showed that use of a cereblon-based E3 ligase reduces the presence of a target protein in a concentration dependent manner.

### Example 8. Effect of Linker Length on E3-Ubiquitin Ligase Induced Proteolysis of GFP

This example illustrates the linker length between the vhhGFP4 (substrate binding domain) and ΔSPOP (ubiquitin pathway moiety) does not significantly affect E3-ubiquitin ligase induced proteolysis of GFP.

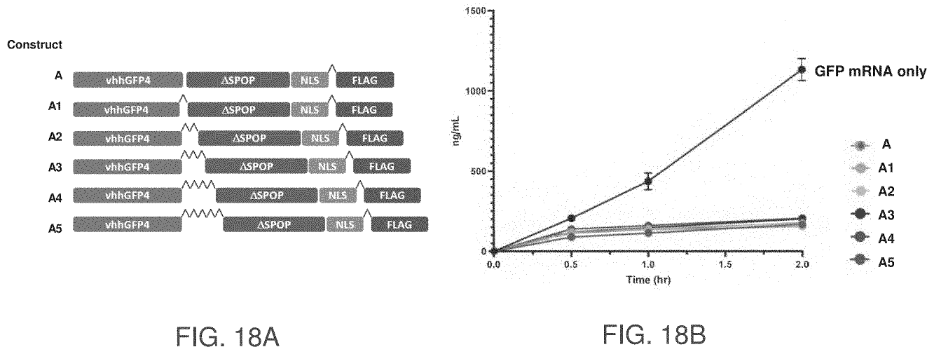

Constructs with various linker length between the vhhGFP4 nanobody and the ΔSPOP E3 ligase were prepared as shown in FIG. 18A and Table 4.

TABLE 4
Variants of construct A with different linker lengths

| | Substrate | | | |
|---|---|---|---|---|
| | binding | | Linker | E3 |
| Construct | domain | Linker | length | Ligase |
| A | vHHGFP4 | — | — | ΔSPOP |
| A1 | vHHGFP4 | GGGS | 4 | ΔSPOP |
| A2 | vHHGFP4 | (GGGS)₂ | 8 | ΔSPOP |
| A3 | vHHGFP4 | (GGGS)₃ | 12 | ΔSPOP |
| A4 | vHHGFP4 | (GGGS)₄ | 16 | ΔSPOP |
| A5 | vHHGFP4 | (GGGS)₅ | 20 | ΔSPOP |

Cytoplasmic extract as described in Example 7 was supplemented with various constructs shown in Table 4 in addition to GFP mRNA. At different time points, the amount of GFP was quantified by ELISA and plotted as shown in FIG. 18B. The results show that the linker length between the vhhGFP4 nanobody and ΔSPOP E3 ligase did not significantly affect the GFP degradation efficiency. All constructs with various linker length were able to effectively reduce amount of GFP in the samples. It is plausible that the degradation induced by the administered construct was so robust that the differential effect of various linker length was not observed in this particular experiment.

### Example 9. Concentration-Dependent Response of E3-Ubiquitin Ligase Induced Proteolysis of A1AT

This example illustrates that the expressed E3-ubiquitin ligases are able to bind its target, A1AT, and induce proteolysis.

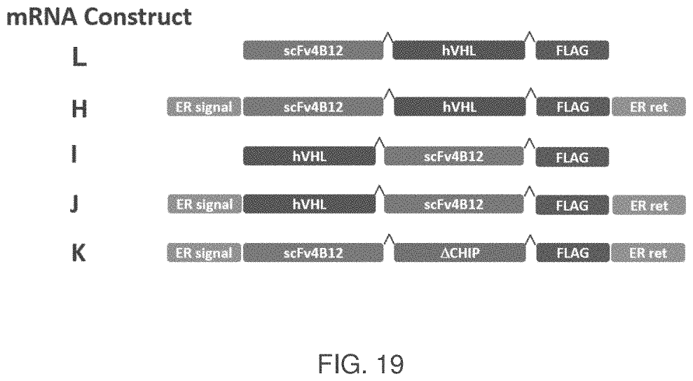

Various mRNA constructs were prepared as shown in FIG. 19. scFv4B12, a single chain variable fragment that specifically recognizes A1AT, was used as a substrate binding domain. In each construct, scFv4B12 was fused to an E3 ligase (hVHL, or ΔCHIP) with a flexible linker (as indicated by \hat{ } in FIG. 19). Each construct was tagged with FLAG, which enables visualization with the anti-FLAG Cy3 dye. Constructs H, J and K further comprises sequences encoding ER signal peptide and ER retention signal. Any number of variations of the above construct can be performed. For example, a linker can be modified, more than one E3 ligase may be used, or a sequence encoding E2 ubiquitin-conjugating enzyme can be introduced. Additionally, different combinations of substrate binding domain, E3 ligase, ER signal peptide, and ER retention signal can be contemplated.

In vitro experiment was performed to examine the dose-response efficacy of E3-ubiquitin ligase encoded by transfected mRNAs on proteolysis of A1AT protein. Cells were co-transfected with 1 μg/1×10⁶ cells of A1AT plasmid and one of constructs G-K (FIG. 19) at various concentrations.

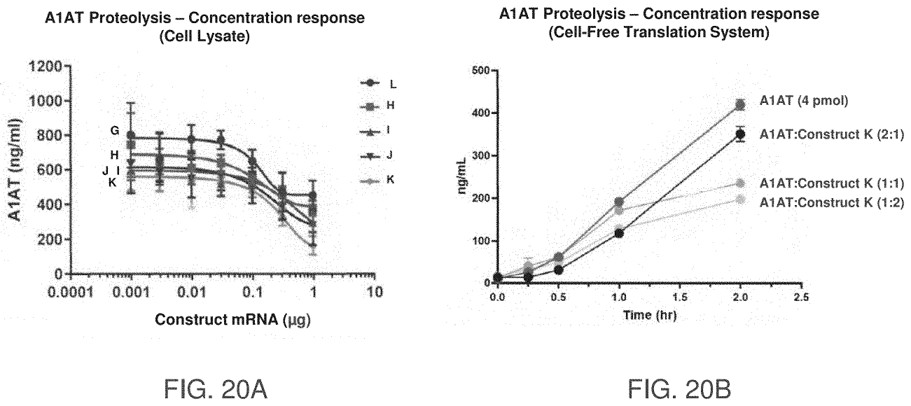

As shown in FIG. 20A, E3-ubiquitin ligases encoded by constructs G-K were able to induce degradation of A1AT in a concentration-dependent manner. In this particular example, degradation of A1AT was observed when mRNA constructs were added at least at 1:1 (Construct mRNA: A1AT plasmid) ratio.

Next, in vitro cell-free translation system was used to study the dose-response efficacy of E3-ubiquitin ligase encoded by transfected mRNAs on proteolysis of A1AT protein. Cytoplasmic extracts were supplemented with 4 pmol of A1AT mRNA and construct K as shown in FIG. 19 at various ratios. As shown in FIG. 20B, sample containing A1AT mRNA alone produced high amount of A1AT. When samples were supplemented with varying amount of construct K, a dose-dependent reduction in A1AT was observed, illustrating that E3-ubiquitin ligase encoded by construct K successfully induced A1AT proteolysis.

### Example 10: bioPROTAC-Mediated Degradation is Driven by the Proteasome

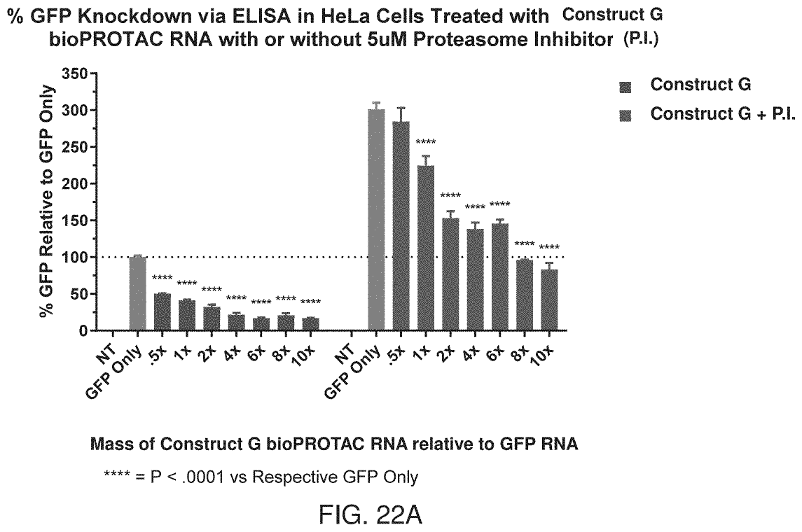

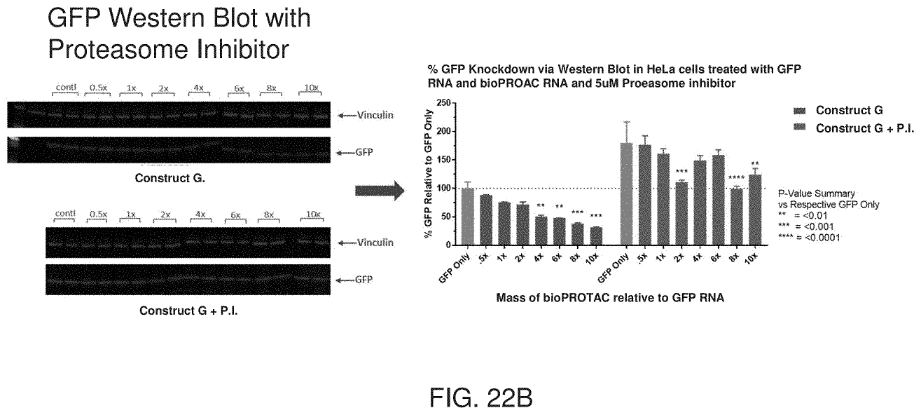

This example shows that bioPROTAC-mediated degradation is driven by the proteasome. For these studies, construct G was used as a representative mRNA construct. To discern the involvement of the proteasome in bioPROTAC-mediated degradation, HeLA cells were administered construct G with or without 5 μM proteasome inhibitor MG-132. Cell isolates were obtained and GFP ELISA was performed. The results of these studies show that GFP is increased in all cells that were treated with MG-132. These data show that construct G resulted in significant proteasomal-dependent degradation of GFP (FIGS. 22A and 22B).

### Example 11: Comparison of Different E3 Ligase bioPROTAC Designs for Targeted Degradation

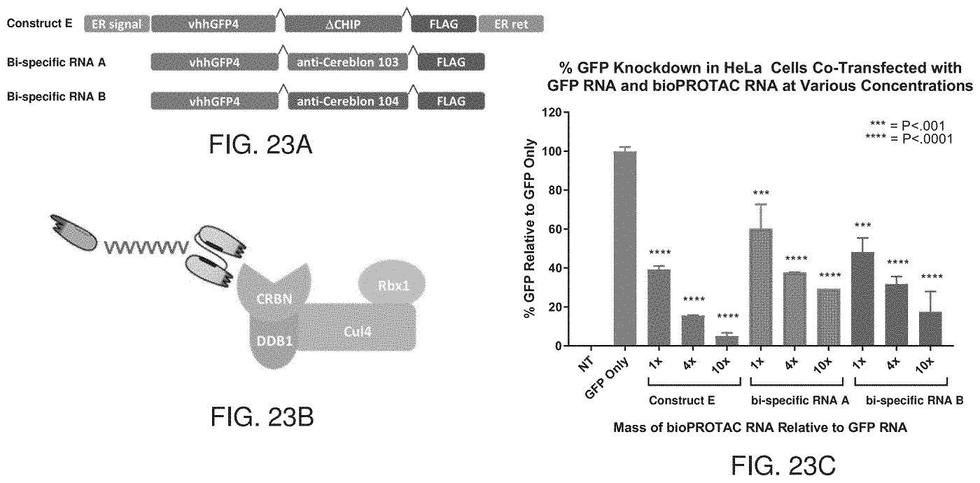

In this example, various E3 Ligase designs were compared for knockdown of a target protein. The designs of the bioPROTACs tested included Construct E and two bi-specific anti-cereblon bioPROTACs (hi-specific RNA A and hi-specific RNA B) (FIG. 23A and FIG. 23B). FIG. 23B is a schematic that shows binding of the bi-specific bioPROTAC to cereblon.

For these studies, HeLa cells were co-transfected with GFP RNA and one of the bioPROTAC designs shown in FIG. 23A. The data from these studies showed that all of the bioPROTAC designs tested caused specific GFP knockdown. These data also show that construct E outperforms each of the anti-cereblon (bi-specific) bioPROTACs in reducing target protein presence (FIG. 23C).

### Example 12: Duration of Expression Study to the Effects of GFP bioPROTACs in Mice

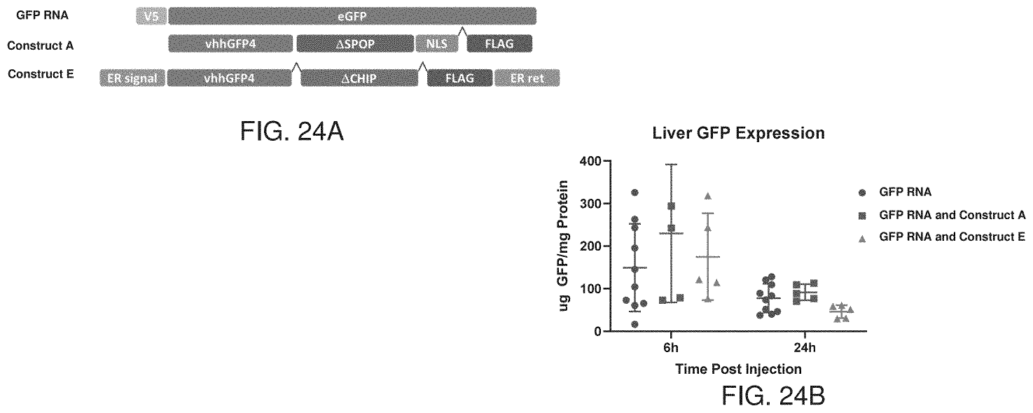

The purpose of the study described in this example was to determine the duration of expression of administered bioPROTACs in mice. The bioPROTACs used in this study are illustrated in FIG. 24A. For these studies, 6-8 week old CD-1 mice were administered by tail vein injection GFP RNA and/or one of the bioPROACTs shown in FIG. 24A. Liver GFP expression was then assessed at 6 hours and at 24 hours post-administration. The data from these studies showed that there was no statistically significant difference in the bioPROTAC treated groups. The data indicate that there is a trend towards reduced liver GFP expression in mice administered construct G (FIG. 24B).

### EQUIVALENTS

Those skilled in the art will recognize or be able to ascertain using no more than routine experimentation, many equivalents to the specific embodiments of the invention described herein. The scope of the present invention is not intended to be limited to the above Description, but rather is as set forth in the following claims:

1. A messenger RNA (mRNA) that encodes a ubiquitin pathway moiety and a binding peptide that binds a target protein, wherein the mRNA is encapsulated within a lipid nanoparticle.

2. The mRNA of claim 1, wherein the ubiquitin pathway moiety and the binding peptide are separated by a linker.

3. The mRNA of claim 2, wherein the linker is a GS linker.

4.-6. (canceled)

7. The mRNA of claim 1, wherein the ubiquitin pathway moiety is an E3 adaptor protein, wherein the E3 adaptor protein is engineered to replace its substrate recognition domain with the binding peptide.

8. The mRNA of claim 7, wherein the E3 adaptor protein is selected from SPOP, CHIP, CRBN, VHL, X1AP, MDM2 and cIAP.

9. The mRNA of claim 1, wherein the ubiquitin pathway moiety is an antibody that specifically binds an E3 adaptor protein, or an E3 ligase.

10. (canceled)

11. The mRNA of claim 1, wherein the binding peptide is an antibody or antibody fragment that specifically binds to the target protein.

12.-13. (canceled)

14. The mRNA claim 11, wherein the target protein is aberrantly expressed in a target cell.

15.-16. (canceled)

17. The mRNA of claim 14, wherein the target protein is an enzyme, a protein involved in cell signaling, cell division, or metabolism, or a protein involved in an inflammatory response.

18. A messenger RNA (mRNA) that encodes at least two binding peptides, wherein a first binding peptide binds an ubiquitin pathway moiety and a second binding peptide binds a target protein, and wherein the mRNA is encapsulated within a lipid nanoparticle.

19. The mRNA of claim 18, wherein the first binding peptide and the second binding peptide are separated by a linker.

20.-21. (canceled)

22. The mRNA of claim 18, wherein the ubiquitin pathway moiety is a ubiquitin pathway protein.

23.-26. (canceled)

27. The mRNA of claim 18, wherein the second binding peptide is an antibody or antibody fragment that specifically binds the target protein.

28.-29. (canceled)

30. The mRNA of claim 27, wherein the target protein is aberrantly expressed in the target cell.

31.-34. (canceled)

35. The mRNA of claim 1, wherein the mRNA further encodes a signal peptide.

36.-39. (canceled)

40. The mRNA of claim 1, wherein the lipid nanoparticle comprises one or more cationic lipids, one or more non-cationic lipids, one or more cholesterol-based lipids and one or more PEG-modified lipids.

41. The mRNA of claim 40, wherein the one or more cationic lipids are selected from the group consisting of cKK-E12, OF-02, C12-200, MC3, DLinDMA, DLinkC2DMA, ICE (Imidazol-based), HGT5000, HGT5001, HGT4003, DODAC, DDAB, DMRTE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, 3-(4-(bis(2-hydroxydodecyl)amino)butyl)-6-(4-((2-hydroxydodecyl)(2-hydroxyundecyl)amino)butyl)-1,4-dioxane-2,5-dione (Target 23), 3-(5-(bis(2-hydroxydodecyl)ainino)pentai1-2-yl)-6-(5-((2-hydroxydodecyl)(2-hydroxyundecyl)amino)pentan-2-yl)-1,4-dioxane-2,5-dione (Target 24), and combinations thereof.

42. The mRNA of claim 1, wherein the target protein comprises a phosphorylated version of the target protein, a non-phosphorylated version of the target protein, a lipidated version of the target protein, a non-lipidated version of the target protein, a pro-peptide version of the target protein, a glycosylated version of the target protein, an unglycosylated version of the target protein, an oxidized version of the target protein, an unoxidized version of the target protein, a carbonylated version of the target protein, a non carbonylated version of the target protein, a formylated version of the target protein, a non-formylated version of the target protein, an acylated version of the target protein, a nonacylated version of the target protein, an alkylated version of the target protein, a non alkylated version of the target protein, a sulfonated version of the target protein, a non sulfonated version of the target protein, an s-nitrosylated version of the target protein, a non s-nitrosylated version of the target protein, a glutathione addition version of the target protein, a non-glutathione addition version of the target protein, an adenylated version of the target protein, a non-adenylated version of the target protein, or an ATP or ADP bound version of the protein, or wherein the target protein is bound to a receptor.

43.-50. (canceled)

51. A method of treating a subject suffering from a disease or disorder associated with aberrant protein expression, comprising administering to the subject in need thereof an mRNA of claim 1, wherein administration of the mRNA results in selective degradation of the aberrantly expressed protein.

52. (canceled)

53. The method of claim 51, wherein the disease or disorder is select from a prion-based disease, polycystic kidney disease, Pelizaeus-Merzbacher disease, an inflammatory disease, and cancer.


SEQUENCE LISTING (25 sequences)

SEQ ID NO: 1 - DNA, 636 bp, Artificial Sequence
  synthetic polynucleotide
agcgtgaacattagcgggcagaataccatgaacatggtcaaagtgccggaatgtcgcctggccgacgaactgggcggcctgtgggaaaactcaaggttcacggactgctgcctttgcgtggccggccaagaattccaggcccataaggccatcctggccgcgcggtcgccagtattctcggccatgttcgaacacgaaatggaagagtctaagaagaatagagtggaaatcaacgatgtggagcctgaggtctttaaggaaatgatgtgctttatatacactggaaaggcccccaacctcgacaagatggccgacgacttgctggctgccgccgacaaatacgccctggagcggctcaaggttatgtgcgaggacgcgctgtgcagcaacctcagcgtggagaacgccgcagaaatcctcatcctggcggatttgcactccgccgaccaactcaagacccaggccgtggacttcattaactaccacgcttccgacgtgctggagacttccggatggaagtccatggtcgtcagccacccgcacttagtggcagaggcctacagatccctggccagtgcccagtgccctttcctggggccgcctaggaaacgcctgaagcagagcgggggtggctcc

SEQ ID NO: 2 - DNA, 675 bp, Artificial Sequence
  synthetic polynucleotide
ggtggtggatccggcggcggctccatgcctaggagagcggagaattgggacgaagcagaagtcggagcagaagaagccggagtggaagaatacggacctgaagaggacgggggagaagagtcgggcgccgaagagtccggccccgaggagtccggacccgaagaactgggcgccgaggaagaaatggaggccgggcgccctaggccggtgctgcggtccgtgaactcccgcgagccgagccaggtcattttctgcaatcgcagcccgagagtggtgctgcccgtgtggctgaactttgacggggagcctcagccataccctaccctgccaccgggaactggacgcagaatccacagctaccggggccacctttggcttttccgggacgccgggactcacgacgggctgctcgtgaaccagaccgagttgttcgtgccgtccctgaacgtcgatggccagccaattttcgccaacatcaccctgccggtgtacacactgaaggaacggtgcctccaagtcgtcagaagcctcgtcaagcccgagaactaccggcggctggacatcgtgcggtcactctacgaagatctcgaggaccaccctaacgtgcaaaaggacctggagaggctgactcaggaacgcatcgcccatcaacgcatgggcgacggtggtggctcc

SEQ ID NO: 3 - DNA, 555 bp, Artificial Sequence
  synthetic polynucleotide
ggttccggctctggacggctgaacttcggggacgatattcctagcgccctgcgcatcgccaagaagaagagatggaactcaatcgaggaacggcgaatccaccaggagtccgagctgcatagctaccttagccgccttatcgccgcggaacgggagagggagctggaagagtgtcagcggaaccatgagggcgacgaagatgactcccacgtccgggcacagcaggcctgcatcgaggctaagcacgacaagtacatggccgatatggacgagttattcagccaagtggacgagaagcggaagaagcgcgacatcccggactacttatgcggaaagatttccttcgaacttatgagggaaccgtgtatcaccccgtccgggatcacctacgaccggaaagacatcgaagaacacctacagcgcgtggggcacttcgacccggtcacccggagcccgctgacccaagagcaattaatccccaacttggcgatgaaggaagtgatcgacgccttcattagcgaaaatggatgggtggaggattacgggggtggctcc

SEQ ID NO: 4 - DNA, 54 bp, Artificial Sequence
  synthetic polynucleotide
ggctggtcttgcattatactcttccttgtcgccaccgccactggagcgcatagc

SEQ ID NO: 5 - DNA, 18 bp, Artificial Sequence
  synthetic polynucleotide
tccgagaaagatgaactg

SEQ ID NO: 6 - protein, 212 aa, Artificial Sequence
  synthetic polypeptide
SVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQSGGGS

SEQ ID NO: 7 - protein, 225 aa, Artificial Sequence
  synthetic polypeptide
GGGSGGGSMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGS

SEQ ID NO: 8 - protein, 185 aa, Artificial Sequence
  synthetic polypeptide
GSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYGGGS

SEQ ID NO: 9 - protein, 18 aa, Artificial Sequence
  synthetic polypeptide
GWSCIILFLVATATGAHS

SEQ ID NO: 10 - protein, 6 aa, Artificial Sequence
  synthetic polypeptide
SEKDEL

SEQ ID NO: 11 - RNA, 140 nt, Artificial Sequence
  synthetic polynucleotide
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 12 - RNA, 105 nt, Artificial Sequence
  synthetic polynucleotide
cggguggcaucccugugaccccuccccagugccucuccuggcccuggaaguugccacuccagugcccaccagccuuguccuaauaaaauuaaguugcaucaagcu

SEQ ID NO: 13 - RNA, 105 nt, Artificial Sequence
  synthetic polynucleotide
ggguggcaucccugugaccccuccccagugccucuccuggcccuggaaguugccacuccagugcccaccagccuuguccuaauaaaauuaaguugcaucaaagcu

SEQ ID NO: 14 - protein, 874 aa, Artificial Sequence
  synthetic polypeptide
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 15 - DNA, 18 bp, Artificial Sequence
  synthetic polynucleotide
atttaggtgacactatag

SEQ ID NO: 16 - DNA, 23 bp, Artificial Sequence
  synthetic polynucleotide
atttaggggacactatagaagag

SEQ ID NO: 17 - DNA, 22 bp, Artificial Sequence
  synthetic polynucleotide
atttaggggacactatagaagg

SEQ ID NO: 18 - DNA, 23 bp, Artificial Sequence
  synthetic polynucleotide
atttaggggacactatagaaggg

SEQ ID NO: 19 - DNA, 20 bp, Artificial Sequence
  synthetic polynucleotide
atttaggtgacactatagaa

SEQ ID NO: 20 - DNA, 22 bp, Artificial Sequence
  synthetic polynucleotide
atttaggtgacactatagaaga

SEQ ID NO: 21 - DNA, 23 bp, Artificial Sequence
  synthetic polynucleotide
atttaggtgacactatagaagag

SEQ ID NO: 22 - DNA, 22 bp, Artificial Sequence
  synthetic polynucleotide
atttaggtgacactatagaagg

SEQ ID NO: 23 - DNA, 23 bp, Artificial Sequence
  synthetic polynucleotide
atttaggtgacactatagaaggg

SEQ ID NO: 24 - DNA, 23 bp, Artificial Sequence
  synthetic polynucleotide
  misc_feature (22)..(22): n is a, c, g, or t
atttaggtgacactatagaagng

SEQ ID NO: 25 - DNA, 24 bp, Artificial Sequence
  synthetic polynucleotide
catacgatttaggtgacactatag